(19) United States
(12) Patent Application Publication
(10) Pub. No.: US 2021/0361673 A1
(43) Pub. Date: Nov. 25, 2021

(54) COMPOSITIONS AND METHODS OF TREATING VIRAL INFECTIONS

(71) Applicant: Lipocine Inc., Salt Lake City, UT (US)

(72) Inventors: Kongnara Papangkorn, Sandy, UT (US); Mahesh V. Patel, Salt Lake City, UT (US); Nachiappan Chidambaram, Sandy, UT (US); Kilyoung Kim, West Jordan, UT (US); Ben Bruno, Salt Lake City, UT (US)

(73) Assignee: Lipocine Inc., Salt Lake City, UT (US)

(21) Appl. No.: 16/875,979

(22) Filed: May 15, 2020

(51) Int. Cl. A61K 31/568; A61K 9/00

(52) U.S. Cl. CPC A61K 31/568; A61K 9/0053

(57) ABSTRACT

The present disclosure is drawn to a methods of treating viral infections using androgen receptor agonists and compositions and dosages suitable therefor.

### FIELD OF THE INVENTION

The present invention relates to methods of treating a subject with a viral infection, such as a Severe Acute Respiratory Syndrome Coronavirus 2 (SARS-CoV-2) infection. Accordingly, this invention involves the fields of chemistry, pharmaceutical sciences, medicine and other health sciences.

### BACKGROUND OF THE INVENTION

Severe Acute Respiratory Syndrome Coronavirus 2 (SARS-CoV-2) is a strain of Severe Acute Respiratory Syndrome Coronavirus (SARSr-CoV) from the family of Coronaviridae. SARS-CoV-2 is a single-stranded RNA virus that causes coronavirus disease 2019 (COVID-19). This virus is highly contagious in humans and causes, among other symptoms, respiratory illness that can ultimately result in death. The virus is primarily spread between people through close contact and via respiratory droplets produced from coughs or sneezes. People may also become infected by touching a contaminated surface and then touching their eyes, nose, or mouth.

Entry of SARS-CoV-2 into cells (e.g., infection) occurs when the virus binds to the angiotensin converting enzyme 2 (ACE2) receptor with assistance of the serine protease TMPRSS2 as a primer. Androgens, like testosterone, may play a role in regulating TMPRSS2 and ACE2 expression. Accordingly, it has been hypothesized that androgen receptor antagonists would be useful in preventing contraction of COVID-19.

While some preliminary approaches are thought to show promise in preventing or reducing contraction of COVID-19, at present no vaccine or approved pharmaceutical treatment is available. As a result, primary efforts to slow and prevent the spread of the SARS-CoV-2 virus have included social distancing (e.g. remaining at least 6 feet away from other individuals whether symptomatic or asymptomatic), quarantine and isolation, the use of virus-filtering masks, and improved hygiene, such as frequent hand washing. Moreover, while a number of treatments for patients infected with COVID-19 have been hypothesized and/or informally attempted, no treatment for reducing or otherwise ameliorating COVID-19 symptoms currently exists. Accordingly, new mechanisms of preventing and treating the COVID-19 condition are being sought.

### SUMMARY OF THE INVENTION

The present disclosure is drawn to a method of treating COVID-19 infection in a subject comprising administering a therapeutically effective amount of a testosterone agent to the subject. In some aspects, the testosterone agent can be testosterone or a testosterone ester selected from the group consisting of: testosterone undecanoate, testosterone dodecanoate, testosterone tridecanoate, and combinations thereof. In some aspects, the testosterone agent can be administered to the subject when COVID-19 symptoms in the subject has reached an acute state, or when COVID-19 symptoms in the subject has reached a chronic state. In some aspects, the subject can display no or substantially no COVID-19 symptoms, mild COVID-19 symptoms, moderate COVID-19 symptoms, severe COVID-19 symptoms, critical COVID-19 symptoms, or acute respiratory distress syndrome symptoms.

The present disclosure is also drawn to a method of treating a virally-induced respiratory condition in a subject comprising administering a therapeutically effective amount of an androgen receptor agonist to the subject. In some aspects, the androgen receptor agonist can be a member selected from the group consisting of: testosterone, a testosterone ester, dehydroepiandrosterone (DHEA), dehydroepiandrosterone sulfate (DHEA-S), androstenedione (A4), Androsterone, androstenediol (A5), nandrolone, dimethandrolone, dihydrotestosterone, and dihydrotestosterone esters. In some aspects, the virally-induced respiratory condition can be a Severe Acute Respiratory Syndrome coronavirus (SARS-CoV-1), a Severe Acute Respiratory Syndrome coronavirus 2 (SARS-CoV-2), a Middle East Respiratory Ryndrome (MERS), influenza, or H1N1. In some aspects, the androgen receptor agonist can be administered to the subject when the virally-induced respiratory or non-respiratory condition in the subject is in an acute state, or when virally-induced respiratory or non-respiratory condition (e.g., due to failure of other organs due to infection) in the subject is in a chronic state. In some aspects, the subject can display no or substantially no respiratory symptoms, mild respiratory symptoms, moderate respiratory symptoms, severe respiratory symptoms, critical respiratory symptoms, or acute respiratory distress syndrome symptoms.

In some aspects, the subject can have a comorbid condition including cardiovascular disease, obesity, diabetes, chronic respiratory disease, hypertension, thyroid dysfunction, chronic kidney disease, liver disease, osteoporosis, hypogonadism, cancer, sleep apnea, frailty, blood coagulation disorder, sarcopenia, cachexia, or combinations thereof. In some aspects, the therapeutically effective amount of the androgen receptor agonist can: improve clinical symptoms associated with lung function, improve clinical outcomes associated with morbidity, or improve clinical outcomes associated with survival, reduce organ deterioration or failure, prevent induction of functional hypogonadism, prevent testicular dysfunction, reduce admission rate in the ICU, and lower sequential organ failure.

### DETAILED DESCRIPTION

Before invention embodiments are described, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples or embodiments only and is not intended to be limiting.

Furthermore, the described features, structures, or characteristics can be combined in any suitable manner in one or more embodiments. In the following description, numerous specific details are provided, such as examples of compositions, dosage forms, treatments, etc., to provide a thorough understanding of various invention embodiments. One skilled in the relevant art will recognize, however, that such detailed embodiments do not limit the overall inventive concepts articulated herein, but are merely representative thereof.

It should be noted that, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

As used herein, the terms “treat,” “treatment,” or “treating” and the like refers to administration of a therapeutic agent to a pathogen-infected subject who is either asymptomatic or symptomatic. In other words, “treat,” “treatment,” or “treating” can refer to reducing, ameliorating or eliminating symptoms associated with a condition present in an infected subject.

As used herein, “SARS-CoV-2” refers to a single-stranded RNA virus that causes coronavirus disease 2019 (COVID-19) along with any SARS-CoV-2 mutations.

As used herein, “androgen receptor agonists” refers to compounds, molecules, or agents such as testosterone that bind and activate an androgen receptor. Examples of androgen receptor agonists include, but are not limited to, dihydrotestosterone, mibolerone, testosterone, alkylated testosterone, derivatives of testosterone esters, methyltrienolone, oxandrolone, nandrolone and fluoxymesterone. Where appropriate, fatty acid esters of these androgen receptor agonists can be used herein accordingly. As used herein, “testosterone ester” refers to testosterone esterified with a fatty acid. Exemplary testosterone esters include without limitation testosterone undecanoate, testosterone decanoate, testosterone dodecanoate, testosterone tridecanoate, testosterone decanoate, testosterone enanthate, testosterone palmitate, testosterone cypionate, and testosterone propionate.

As used herein, “testosterone agent” refers to an active pharmaceutical agent that produces a physiologic action or effect of testosterone in-vivo. Examples of testosterone agents are found throughout the present application. One example of a testosterone agent is testosterone (T). Another example is a testosterone ester, such as testosterone undecanoate or testosterone tridecanoate.

As used herein, the terms “therapeutic agent,” “active agent,” and the like can be used interchangeably and refer to agent that can have a beneficial or positive effect on a subject when administered to the subject in an appropriate or effective amount. In one aspect, the therapeutic or active agent can be an androgenic steroid. The terms “additional active agent,” “supplemental active agent,” “secondary active agent,” and the like can be used interchangeably and refer to a compound, molecule, or material other than an androgenic steroid that has physiologic activity when administered to a subject in an effective amount.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format for administration to a subject. For example, an “oral dosage form” can be suitable for administration to a subject's mouth. A “topical dosage form” can be suitable for administration to a subject's skin by rubbing, etc.

As used herein, a “subject” refers to an animal. In one aspect the animal may be a mammal. In another aspect, the mammal may be a human.

As used herein, “in need of treatment” refers to a subject that has a disease or is suspected of having the disease according to various diagnostic criteria typically used in practice, or desires treatment or is indicated for treatment. Thus, “in need of treatment” can include the step of identifying a subject in need of treatment.

As used herein, “identifying a subject in need of treatment” can include the step of obtaining a biological sample from the subject and determining the level of one or more biomarkers as described herein (RT-PCR detection of a virus gene), assessing a biological sample obtained from said subject, performing an imaging analysis on the subject, assessing one or more clinical characteristics of said subject (e.g., assessing symptoms or overt symptoms), or a combination thereof.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, an asthma attack, bronchitis, a heart attack, pneumonia, and the like. Some examples of chronic conditions can include without limitation, arthritis, diabetes, hypertension, high cholesterol, and the like.

The terms “serum testosterone” or “serum (17-β)-Hydroxy-4-Androsten-3-one levels,” “serum T levels,” “serum testosterone concentration,” “plasma testosterone concentration,” “testosterone concentration in the blood,” and “serum testosterone concentration,” are used interchangeably and refer to the “total” serum testosterone concentration which is the sum of the bioavailable testosterone including free and bound testosterone fractions or concentrations. As with any bioanalytical measure, for increased consistency, the method employed to measure initial serum testosterone levels may be consistent with the method used to monitor and re-measure serum testosterone levels during clinical testing and testosterone therapy for a subject. Unless otherwise stated, “testosterone concentration” refers to serum total testosterone concentration.

Average serum testosterone concentrations can be determined using methods and practices known in the art. For example, the average baseline plasma testosterone concentration of a human male is the arithmetic mean of the total plasma testosterone concentration determined on at least two consecutive time points that are reasonably spaced from each other, for example from about 1 hour to about 168 hours apart. In a particular case, the plasma testosterone concentration can be determined on at least two consecutive times that are about 12 hours to about 48 hours apart. In another particular method, the plasma testosterone concentration of the human male can be determined at a time between about 5 o'clock and about 11 o'clock in the morning. Further, the plasma testosterone concentration can be the determined by standard analytical procedures and methods available in the art, such as for example, automated or manual immunoassay methods, liquid chromatography or liquid chromatography-tandem mass spectrometry (LC-MSMS) etc.

As use herein with respect to physiologic levels of a given substance, the term “baseline” refers to a level or concentration of the substance in a subject prior to administration of an active agent. For example, the baseline level of serum testosterone in a subject would the subject's testosterone serum level prior (e.g. just prior) to the commencement of testosterone administration or therapy.

As used herein, “free testosterone serum concentration”, refers to the fraction of total testosterone that is not bound to a protein e.g., SHBG or albumin. In some aspects of the methods described herein, free testosterone serum concentrations are used instead of serum total testosterone concentrations. For example, a subject can appear to have total serum testosterone levels in the normal range, but can be still considered testosterone deficient based on free testosterone levels. It should be understood that as used herein, the term testosterone serum levels or concentrations can provide express support for free testosterone serum concentrations, unless the context or recitation clearly dictates otherwise.

The term “oral administration” represents any method of administration in which an active agent can be administered by swallowing, chewing, or sucking of the dosage form. Oral administration can be intended for enteral delivery of an active agent or transmucosal delivery of the active agent. In some embodiments, the composition of the current inventions can be admixed with food or drink prior to being orally consumed.

As used herein, a “dosing regimen” or “regimen” such as an “initial dosing regimen” or “starting dose” or a “maintenance dosing regimen” refers to how, when, how much, and for how long a dose of the compositions of the present invention can be administered to a subject. For example, an initial or starting dose regimen for a subject may provide for a total daily dose of from about 15 mg to about 1500 mg administered in two divided doses at least 12 hours apart (e.g. once with breakfast and once with dinner) with meals repeated daily for 30 days.

As used herein, “daily dose” refers to the amount of active agent (e.g. a testosterone ester) administered to a subject over a 24-hour period of time. The daily dose can be administered one or more administrations during the 24-hour period. In one embodiment, the daily dose provides for two administrations in a 24-hour period. With this in mind, an “initial dose” or initial daily dose” refers to a dose administered during the initial regimen or period of a dosing regimen.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein “single unit” when used to describe dosing of a subject refers to the dosage form being a single dosage form, e.g. a single tablet, capsule, pump or squirt of gel or solution, etc. In contrast, “multiple unit” when used to describe dosing of a subject refers to the dosage including two or more dosage forms, e.g. 2 capsules, 3 tablets, 2-4 pumps or squirts, etc. It is noteworthy that multiple unit dosage forms generally will be the same type of dosage forms (i.e. tablet or capsule) but are not required to be the same dosage form type.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art. For example, a composition that “increases” testosterone serum levels provides a testosterone serum level in a subject that is elevated as compared to a serum level at a previous point in time, such as a baseline level (e.g. prior to treatment), or as compared to an earlier treatment with a different (e.g. lower dose).

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, variants, modifications, and equivalents as may be included within the spirit and scope of the invention as defined by the appended claims.

The severe acute respiratory syndrome coronavirus 2 (SARS-Cov-2), the pathogen of 2019 novel coronavirus disease (COVID-19), poses a serious threat to global public health. Besides pneumonia, COVID-19 can also cause damage to organs such as the heart, liver, and kidneys, as well as to organ systems such as the blood and the immune system. Some subjects eventually die of multiple organ failure, shock, acute respiratory distress syndrome, heart failure, blood clots, arrhythmias, and renal failure. Liver injury has also been reported in 14.8% to 53% of severe cases. Incidence of liver injury is mainly indicated by abnormal ALT/AST levels accompanied by slightly elevated bilirubin levels.

The potential mechanisms underlying COVID-19 organ damage can involve a massive cytokine production (i.e. cytokine storm) followed by organ crosstalk. For example, COVID-19 can have fatal consequences for subjects with cardiovascular disease and can cause cardiac injury even in subjects without heart conditions. Up to half of hospitalized subjects had increased liver enzyme levels compared to baseline levels, which indicates a struggling liver. In fact, the cytokine storm and associated medications administered to treat the virus may be causing liver damage. Also, some subjects with severe COVID-19 have had kidney damage or failure, as well as testicle malfunction resulting in decreased total testosterone levels or reduced reproduction function.

COVID-19 disproportionately affects male subjects—male subjects have had higher rates of hospitalization, intensive care unit (ICU) admissions, and account for up to 80% of COVID-19 related deaths. Moreover, disease severity and fatality directly relate to the age of these male subjects. For example, during one outbreak in Italy, 83% of COVID-19 infected subjects were over the age of 60 with possibly compromised immune reactions. In some cases, the seriousness of the COVID-19 illness appears to involve an overheated immune system leading to a cytokine storm. A cytokine storm can result in a hyper inflammatory state (e.g., Acute Respiratory Distress Syndrome).

Subjects over the age of 65 and adults with comorbid conditions cannot adequately respond to the hyper inflammatory state which can result in death. Baseline low grade inflammation in subjects with functional hypogonadism associated with comorbidities can exacerbate the “cytokine storm” due to low testosterone. Moreover, infected macrophages can trigger cytokine release in the adipose tissue, which may be dependent on the subject's viral load. The Center for Disease Control (CDC) has reported that COVID-19 subjects with a certain comorbidity yielded severe conditions at a higher rate than those subjects without a comorbidity. A research group reported the hazard ratio (HR) was 1.79 among subjects with at least one comorbidity and 2.59 among subjects with two or more comorbidities. After adjusting for age and smoking status, the HR of single comorbidity to the composite endpoints was 2.68 (COPD), 1.59 (diabetes), 1.58 hypertension, and 3.50 (malignancy).

Among laboratory-confirmed cases of COVID-19, subjects with any comorbidity had poorer clinical outcomes than those subjects without any comorbidity. A greater number of comorbidities such as cardiovascular disease, obesity, diabetes, chronic respiratory disease, and hypertension, also correlated with poorer clinical outcomes, possibly due to the chronic inflammation associated with these comorbidities.

Low testosterone might result in acute frailty which can be a contributory factor to fatality. As such, testosterone therapy may be beneficial in afflicted patients because of its anabolic properties. Total and free testosterone levels and diurnal variation in healthy men can progressively decline substantially (>30% from peak levels) as men age. Lower T levels in men are associated with clinically-relevant alterations in biomarkers associated with poor prognosis (increased levels of inflammatory cytokines, lactate dehydrogenase, ferritin, and procalcitonin, decreased lymphocyte count, and increased neutrophil count). Additionally, hypogonadism is observed in the majority of older men who exhibit severe disease, Acute Respiratory Distress Syndrome (ARDS), clinically deteriorate, and/or die. One study estimated that men with a T level below 5 nmol/L (144 ng/dL) can have a 20-30× higher risk of poor outcomes compared to men with a T level greater than 5 nmol/L (144 ng/dL). Furthermore, as observed with other infections, COVID-19 infections may repress testosterone production in men by adversely affecting the hormone axis with testis, thereby exacerbating the intense inflammatory response.

Certain viruses like members of the Coronaviradae have been shown to use the ACE2 receptor and the TMPRSS2 serine protease to enter cells where viral replication occurs. As androgens induce the expression of both ACE2 and TMRPSS2, androgen receptor blockers, TMPRSS2 blockers, and TMPRSS2 down regulators have been shown to decrease the infectivity of coronaviruses in model systems. Androgen receptor blockers are either in clinical trials for COVID-19 or have been proposed as therapeutic agents for COVID-19. In part due to the greater degree of disease severity/mortality in COVID-19 male subjects compared to female subjects, it has been proposed that the sex-based differences in serum androgen levels (e.g., testosterone) explain this phenomena.

Therefore, it is unexpected that androgen receptor agonists can be used to treat subjects infected with coronavirus because androgens, such as testosterone, upregulate ACE2 and TMPRSS2 expression. In some embodiments of the present disclosure, androgen receptor agonists (e.g., testosterone and derivatives of testosterone esters) can beneficially modulate biochemical and physiological pathways associated with poor outcomes in subjects infected with coronavirus. Therefore, provided herein are methods for treating viral infections like coronavirus (e.g. COVID-19) without concomitantly worsening outcomes, or in the alternative, improving disease outcomes. In a specific non-limiting implementation of the methods provided in this disclosure, a human male subject infected by SARS-CoV-2 or having COVID-19 can be administered, for example, orally administered, a therapeutically effective amount of a testosterone agent to provide an improvement in a clinical outcome in the subject.

In some aspects, the methods and compositions described herein exert unexpected effects in liver and related diseases because they are delivered orally e.g., perorally, and absorbed via the intestinal lymphatic system. It is contemplated that delivery via the intestinal lymphatic system can provide surprising results due to one or more of: (a) effects in the lymphatic or interstitial compartment, or (b) improved delivery to target tissues or cells (e.g., one or more of fat, adipocytes, preadipocytes, liver, liver cells, hepatocytes, adipocytes, white blood cells, red blood cells, stem cells, bone, bone cells, androgen receptor-positive cells, androgen receptor-negative cells, cells involved in glucose metabolism, cells involved in the pulmonary system, pneumocytes, bronchial cells, epithelial cells, goblet cells, ciliated cells, basal cells, endothelial cells, type I pneumocytes, type II pneumocytes, macrophages, and the like). Thus, in some specific aspects, delivery of the compositions via the methods disclosed herein can result in transport of the active agent (e.g., testosterone ester) via intestinal lymphatics, chylomicrons, or chylomicron remnants to specific target tissues and cells to provide the unexpected results described herein.

Traditionally testosterone therapy has been primarily indicated for chronic conditions such as hypogonadism. However, testosterone therapy has been heretofore unused for treatment of an acute condition. Treating an acute condition with testosterone can be viewed as unlikely to result in success because testosterone therapy can take weeks to have an effect on testosterone serum levels.

Nonetheless, in some aspects, oral testosterone therapy in subjects with COVID-19 can lead to improved clinical outcomes in patients with moderate symptoms. In one mechanism, immunomodulation and anti-inflammatory properties can stimulate erythrocyte production thereby improving the oxygen carrying capacity of the blood and endothelial function as well as lowering levels of undesirable excess reactive oxygen species (ROS). In another mechanism, anabolic effects of testosterone can improve strength and reduce frailty which can have direct effects on the smoother muscle of the airway, especially in subjects with weakened lung function. In another mechanism, improvement in liver health associated with reductions of elevated liver enzymes and fat can restore testosterone (T) levels in patients with endocrine disorder due to the infection and counter the negative impact on T production used to minimize potential organ failure. In another mechanism, Oral T therapy for acute states and/or chronic states of COVID-19 can align with natural diurnal variation in T and lower sex hormone-binding globulin (SHBG), thereby leading to higher free T and androgen activity.

In one embodiment, a method of treating a virally-induced respiratory condition (e.g., COVID-19) in a subject can comprise administering a therapeutically effective amount of an androgen receptor agonist (e.g., a testosterone agent) to the subject. In some aspects, the virally-induced respiratory condition can be severe acute respiratory syndrome coronavirus (SARS-CoV-1), severe acute respiratory syndrome coronavirus 2 (SARS-CoV-2), middle east respiratory syndrome (MERS), influenza, or H1N1. In another aspect, the viral infection can be selected from the group of families including: Coronoviridae, Orthomyxoviridae, Paramyxoviridae, Picornaviridae, Adenoviridae, and Parvoviridae. In another aspect, the virus can be SARS-CoV-1, SARS-CoV-2, MERS, or influenza. In another embodiment, the methods applied herein can treat respiratory conditions including one or more of acute respiratory distress, pneumonia, lung fibrosis, idiopathic pulmonary fibrosis, chronic obstructive pulmonary disease, and elevated respiratory rate.

In another aspect, influenza can be any of Influenza A, Influenza B, Influenza C, and Influenza D. In another aspect, the virus can be an adenovirus, parainfluenza, rhinovirus, cytomegaloviruses, or a coronavirus. In one aspect, the virus is a virus chosen from the order Nidovirale. In one aspect, the virus is chosen from the alpha, beta, gamma or delta genera of the Nidovirale order. In one aspect, the virus is an RNA virus. In one aspect, the virus is a DNA virus. In one aspect, the virus is a positive sense RNA virus. In one aspect, the virus is a negative sense RNA virus.

In one embodiment, the androgen receptor agonist can be a member selected from the group consisting of: testosterone, methyl testosterone, a testosterone ester, dehydroepiandrosterone (DHEA), dehydroepiandrosterone sulfate (DHEA-S), androstenedione (A4), Androsterone, androstenediol (A5), nandrolone, dimethandrolone, dihydrotestosterone, dihydrotestosterone esters, and combinations thereof.

In one aspect, the androgen receptor agonist (e.g., testosterone agent) can be administered to the subject in an acute-care setting, or in a chronic-care setting after administration in the acute-care setting. In another aspect, the androgen receptor agonist (e.g., testosterone agent) can be administered to the subject in an acute state or acute disease state, or in a chronic-care state or chronic disease state. In some examples, the subject can have a comorbid condition including lung disease, cardiovascular disease, obesity, dyslipidemia, diabetes, chronic respiratory disease, hypertension, thyroid dysfunction, chronic kidney disease, liver disease, osteoporosis, hypogonadism, cancer, sleep apnea, frailty, blood coagulation disorder, sarcopenia, cachexia, and combinations thereof.

The subject (e.g. infected subject) can be asymptomatic or symptomatic with relation to respiratory symptoms (e.g., COVID-19 symptoms). In one example, the subject can display no or substantially no symptoms, such as COVID-19 symptoms, but can be infected with a virus (e.g., SARS-CoV-2). A positive test for a virus (e.g., SARS-CoV-2) in the absence of symptoms can be determined by standard reverse transcription polymerase chain reaction (RT-PCR) assay or equivalent test. In another aspect, the subject can be diagnosed with a virally-induced respiratory condition using an antibody test, a lung CT scan, or other clinical assessment. In another aspect, the subject can have a low viral load. In one aspect, the subject can be receiving supplemental oxygen. In one aspect, the subject can have low blood oxygen levels. In one example, the subject can be asymptomatic or symptomatic in conjunction with a standard of care of based on use of other pharmacological agents.

In another example, the subject can display only mild to respiratory symptoms (e.g., COVID-19 symptoms) which can be indicative of infection with a virus (e.g., SARS-CoV-2). In one aspect, mild respiratory symptoms can include one or more of fever, cough, sore throat, malaise, headache, muscle pain, or gastrointestinal symptoms, without shortness of breath or dyspnea, or clinical signs indicative of moderate, severe, or critical severity.

In another example, the subject can display moderate respiratory symptoms (e.g., COVID-19 symptoms) which can be indicative of infection with a virus (e.g., SARS-CoV-2). In one aspect, symptoms of moderate respiratory illness can include any symptom of mild illness and shortness of breath with exertion. In one aspect, clinical signs indicative of moderate respiratory illness (e.g., COVID-19 illness) can include one or more of: a respiratory rate greater than or equal to 20 breaths per minute, a saturation of oxygen (SpO2) less than or equal to 93% on room air at sea level, or a heart rate greater than or equal to 90 beats per minute without any clinical signs indicative of severe or critical illness severity.

In another example, the subject can display severe respiratory symptoms (e.g., COVID-19 symptoms) which can be indicative of infection of a virus (e.g., SARS-CoV-2). In one aspect, symptoms of severe respiratory illness (e.g., COVID-19 illness) can include any symptom of moderate illness, shortness of breath at rest, or respiratory distress. In one aspect, clinical signs indicative of severe respiratory illness (e.g., COVID-19 illness) can include one or more of: a respiratory rate greater than or equal to 30 per minute, a heart rate greater than or equal to 125 per minute, or a SpO2 less than or equal to 93% on room air at sea level, or a 468 PaO2/FiO2 less than 300 without any criteria for critical severity.

In another example, the subject can display critical respiratory symptoms (COVID-19 symptoms) which can be indicative of infection of a virus (e.g., SARS-CoV-2). In one aspect, symptoms of critical respiratory illness (e.g., COVID-19 illness) can include respiratory failure based on resource utilization using at least one of: (a) endotracheal intubation and mechanical ventilation, (b) oxygen delivered by high-flow nasal cannula (heated, humidified, oxygen delivered via reinforced nasal cannula at flow rates >20 L/min with fraction of delivered oxygen ≥0.5), (c) noninvasive positive pressure ventilation, (d) ECMO, (e) clinical diagnosis of respiratory failure (i.e., clinical need for one of the preceding therapies, but preceding therapies not able to be administered in setting of resource limitation), (f) shock (defined by systolic blood pressure less than 90 mm Hg, or diastolic blood pressure less than 60 mm Hg or requiring vasopressors), or (g) multi-organ dysfunction or failure.

In another example, the subject can display acute respiratory distress syndrome (ARDS) or pneumonia.

In another embodiment, the method can further comprise administering the therapeutically effective amount of the androgen receptor agonist (e.g., testosterone agent) to the subject according to a dosage regimen. In one aspect, the dosage regimen can include administering the therapeutically effective amount of the androgen receptor agonist (e.g., testosterone agent) at least once per day for a specified duration from about a single day to about 1 month, 2 months, 3 months, or 6 months.

In another embodiment, the androgen receptor agonist (e.g., testosterone agent) can be administered to the subject using a route that can result in a substantial increase in a ratio of free androgen serum levels (e.g., testosterone serum levels) to total androgen serum levels (e.g., testosterone serum levels), wherein free T is a component of total T and free T increases by a greater proportion. In one aspect, the androgen receptor agonist (e.g., testosterone agent) can be administered to the subject using a route of administration selected from: oral, transdermal, nasal, injectable, topical, or combinations thereof. In another example, the androgen receptor agonist (e.g., testosterone agent) can be administered to the subject using a route that results in a substantial increase in free testosterone serum levels and a concurrent substantial decrease in sex hormone binding globulin (SHBG). In another example, the androgen receptor agonist (e.g., testosterone agent) can be administered to the subject using a route that results in a substantial decrease in sex hormone binding globulin (SHBG).

In another example, the androgen receptor agonist (e.g., testosterone agent) can be administered to the subject in an acute state (e.g., acute COVID-19 symptoms). In this example, the androgen receptor agonist (e.g., testosterone agent) can be administered to the subject using a suitable route of administration (e.g., transdermal, injectable, topical, or combinations thereof). In another embodiment, the method can further comprise administering the therapeutically effective amount of the androgen receptor agonist (e.g., testosterone agent) to the subject in an acute state according to a dosage regimen. In one aspect, the dosage regimen can include administering the therapeutically effective amount of the androgen receptor agonist (e.g., testosterone agent) at least once per day for a specified duration from about a single day to about 1 month, 2 months, 3 months, or 6 months.

In another embodiment, the androgen receptor agonist (e.g., testosterone agent) can be orally administered to the subject. Oral administration of the androgen receptor agonist (e.g., the testosterone agent) can include differentiation of SBHG, mRNA IL-6, IL-10, CD68, SREBP1—which can occur with oral T. In one aspect, the therapeutically effective amount of the androgen receptor agonist (e.g., testosterone agent) can be orally administered once per day or twice per day. In another aspect, the therapeutically effective amount of the androgen receptor agonist (e.g., testosterone agent) can be orally administered with food.

In another embodiment, the androgen receptor agonist (e.g., the testosterone agent) can be derived from a testosterone ester or a combination of testosterone esters. In one aspect, the testosterone ester can be a member of a group consisting of: testosterone undecanoate, testosterone dodecanoate, testosterone tridecanoate, and combinations thereof.

In another embodiment, a daily dose of the therapeutically effective amount of the testosterone ester can comprise from about 150 mg to about 1500 mg of the testosterone ester when the testosterone ester is testosterone undecanoate and the subject is male. In another aspect, a daily dose of the therapeutically effective amount of the testosterone ester can comprise from about 10 mg to about 150 mg of the testosterone ester when the testosterone ester is testosterone undecanoate and the subject is female. In another aspect, a daily dose of the therapeutically effective amount of the testosterone ester can comprise from about 200 mg to about 1800 mg of the testosterone ester when the testosterone ester is testosterone dodecanoate and the subject is male. In another aspect, a daily dose of the therapeutically effective amount of the testosterone ester can comprise from about 15 mg to about 180 mg of the testosterone ester when the testosterone ester is testosterone dodecanoate and the subject is female. In another aspect, a daily dose of the therapeutically effective amount of the testosterone ester can comprise from about 300 mg to about 2700 mg of the testosterone ester when the testosterone ester is testosterone tridecanoate and the subject is male. In another aspect, a daily dose of the therapeutically effective amount of the testosterone ester can comprise from about 20 mg to about 270 mg of the testosterone ester when the testosterone ester is testosterone tridecanoate and the subject is female. In another example, the subject can have an age greater than one or more of 18, 30, 40, 50, 55, 60, 65, 70, 75, and 80 years old.

In another embodiment, the therapeutically effective amount of the testosterone agent can result in a maintenance or increase in a level of a T/17-β-estradiol (E2) ratio compared to a baseline level of the T/E2 ratio established prior to or at commencement of the testosterone therapy.

In another embodiment, the therapeutically effective amount of the testosterone agent can increase the subject's serum testosterone levels prior to start of the therapy or e.g., into 2×, 3×, 4× . . . 10× range of androgen pre-treatment level for from 0-2 hours, 2-4 hours, 4-6 hours, 6-8 hours, 8-10 hours, 10-12-hour, 12-14 hours, 14-18 hours, 18-20 hours, 20-22 hours, or 22-24 hours per day.

In another embodiment, the androgen receptor agonist (e.g., the testosterone agent) can be in a dosage form selected from an oral dosage form, a topical dosage form, a transdermal dosage form, a nasal dosage form, a parenteral dosage form, a nebulizer dosage form, and combinations thereof.

In another example, the androgen receptor agonist (e.g., the testosterone agent) can be in a dosage form comprising one or more of: a drink, granules, sprinkles, apple sauce, and liquid. In one example, the androgen receptor agonist (e.g., the testosterone agent) can be administered via intubation.

Although some of the description focuses on the oral administration of testosterone esters, the methods and compositions described herein can be adapted to other pharmaceuticals such as androgen agonists, androgens, androgenic-anabolic compounds, selective androgen receptor modulators and other compounds that target androgen receptor signaling pathways.

In another embodiment, the method can further comprise identifying a subject in need of treatment. In aspects of the methods described herein, the subject in need of treatment can have a viral infection or symptom thereof and can have testosterone deficiency or low testosterone. In one aspect, said subject in need of treatment has testosterone deficiency or low testosterone with serum testosterone levels of less than 500 ng/dL, 450 ng/dL, 400 ng/dL 350 ng/dL, 300 ng/dL, 250 ng/dL, 200 ng/dL, 150 ng/dL or 100 ng/dL.

In another embodiment, the subject in need of treatment can have a diagnosis of infection by a respiratory virus. In one aspect, the subject can have a diagnosis of infection by a respiratory virus as determined by RT-PCR. In one aspect, the subject can have a diagnosis of infection by a respiratory virus as determined by an antibody test. In one aspect, the respiratory virus can be chosen from SARS-COV1, SARS-COV2, MERS, influenza, or H1N1.

In another embodiment, the subject in need of treatment can have a diagnosis of a panel of hormones comprising cortisol, thyroid-stimulating hormone (TSH), free triiodothyronine (T3), free thyroxine (T4), luteinizing hormone (LH), follicle-stimulating hormone (FSH), total testosterone (TT), dihydrotestosterone (DHY-TT), 17-β-estradiol (E2) and sex hormone binding globulin (SHBG). In another embodiment, the subject in need of treatment can have a primary or secondary gonadal dysfunction as a comorbid condition or as a result of a viral infection.

In another embodiment, the therapeutically effective amount of the androgen receptor agonist can improve clinical symptoms associated with lung function (e.g., improving or treating lung function). In another embodiment, the therapeutically effective amount of the androgen receptor agonist can improve clinical outcomes associated with morbidity. In another embodiment, the therapeutically effective amount of the androgen receptor agonist can improve clinical outcomes associated with survival. In one aspect, improved clinical outcomes can include one or more of: reducing the risk of death, reducing the progression of mild disease to severe disease leading to intensive care, reducing the need for mechanical or non-mechanical ventilation, reducing the length of time a subject experiences symptoms, increasing the rate of recovery from symptoms, reducing the risk of experiencing acute respiratory distress, reducing or lessening the progression of lung disease as monitored by lung CT scans, biomarkers, and the like.

In another embodiment, a method of minimizing an inflammatory or pro-inflammatory cytokine mRNA expression in organs or serum cytokine levels in a subject can comprise administering a therapeutically effective amount of an androgen receptor agonist to the subject. In another embodiment, the therapeutically effective amount of the androgen receptor agonist (e.g., testosterone agent) can result in a decrease of pro-inflammatory cytokines and pro-inflammatory chemokines. In one aspect, the pro-inflammatory cytokines can consist of: interleukin (IL-1), Il-1 beta, IL-6, IL-8, IL-12, gamma interferon (IFN-γ), IL-18, tumor necrosis factor (TNF). In another aspect, the pro-inflammatory chemokines can consist of the monocyte chemoattractant protein-1 (MCP-1).

In certain embodiments, provided herein is a pharmaceutical composition comprising at least one steroidal compound (e.g., testosterone, dihydrotestosterone, estradiol, or analogs or prodrugs thereof) and at least one pharmaceutically acceptable carrier. In specific embodiments, the steroidal compound is a steroidal androgen (e.g., testosterone, dihydrotestosterone, or prodrugs thereof). In some embodiments, the steroidal compound is an alkylated, hydroxy-alkylated and/or hydroxy-alkoylated natural steroid (e.g., testosterone alkyl ester, dihydrotestosterone alkyl ester, estradiol alkyl ester, or the like). In certain embodiments, analogs or prodrugs of testosterone include, e.g., esters of testosterone. In specific embodiments, the esters of testosterone include, e.g., alkyl (e.g., straight chain, branched, cyclic, unsaturated, partially saturated, fully saturated and the like) esters of testosterone. Specifically, alkyl esters of testosterone include, by way of non-limiting example, lower alkyl esters (e.g., testosterone C2-C13 alkyl esters such as testosterone propionate, testosterone enanthate, testosterone undecanoate, testosterone dodecanoate, or testosterone tridecanoate), or higher alkyl esters (e.g., testosterone C14+ alkyl esters such as testosterone palmitate). In further embodiments, the alkyl esters of testosterone include, by way of non-limiting example, cycloalkyl esters (e.g., testosterone cypionate), and alkylcycloalkyl esters. In more specific embodiments, the testosterone alkyl ester is testosterone undecanoate. In more specific embodiments, the testosterone alkyl ester is testosterone tridecanoate. In more specific embodiments, the testosterone alkyl ester is testosterone dodecanoate. In some embodiments, the at least one steroidal compound comprises (1) a testosterone lower alkyl ester (e.g., testosterone propionate, testosterone enanthate, or testosterone undecanoate); and (2) a testosterone higher alkyl ester (e.g., testosterone palmitate). Generally, as used herein, a pharmaceutical composition comprising a steroidal compound includes the disclosure of a pharmaceutical composition comprising one or more steroidal compounds.

In some embodiments, dihydrotestosterone or a dihydrotestosterone ester can be used in place or in conjunction with a testosterone ester as described herein. Exemplary dihydrotestosterone esters include, but are not limited to, dihydrotestosterone propionate, dihydrotestosterone enanthate, dihydrotestosterone cypionate, dihydrotestosterone undecanoate, dihydrotestosterone decanoate, dihydrotestosterone dodecanoate, dihydrotestosterone tridecanoate, dihydrotestosterone tetradecanoate, and dihydrotestosterone palmitate.

In certain embodiments, any pharmaceutical composition described herein comprises a therapeutically effective amount of at least one steroidal compound (e.g., a testosterone alkyl ester, such as testosterone undecanoate). In some embodiments, a therapeutically effective amount of a steroidal compound (e.g., a testosterone alkyl ester, such as testosterone undecanoate) is divided into one or more oral dosage form. In some embodiments, the one or more of the oral dosage forms described herein collectively comprise a therapeutically effective amount of a testosterone alkyl ester (e.g., testosterone undecanoate). Thus, in some embodiments, the therapeutically effective amount of a steroidal compound (e.g., a testosterone alkyl ester, such as testosterone undecanoate) within a pharmaceutical composition described herein may vary when the pharmaceutical composition is administered in combination with another therapy. Furthermore, therapeutically effective amounts of a formulation may depend on the specific formulation within which the at least one steroidal compound is found. For example, in some embodiments, more than one steroidal compound is present in a pharmaceutical composition described herein. Thus, when there is a combination of steroidal compounds, in certain instances one or both of the steroidal compounds present has a therapeutically effective amount that is lower than is required when the steroidal compounds are administered separately or alone. In some embodiments, a pharmaceutical composition described herein further comprises an adjuvant, which, in certain instances, allows for a lower amount of a steroidal compound to be utilized as a therapeutically effective amount.

As described in typical total daily dose ranges for adult male and female subjects are given for testosterone undecanoate and testosterone tridecanoate. Based on the doses, doses for other testosterone esters can be estimated on the T-equivalent dose which is determined e.g., by the amount of testosterone per testosterone undecanoate molecule. For example, for calculation purposes, 1 mg of T is equivalent to: 1.39 mg T-enanthate; 1.58 mg T-undecanoate; 1.43 mg T-cypionate and so on. Although the conversion is not exact due to various properties, a skilled artisan can understand how to estimate a dose of one testosterone ester for another testosterone ester based on this calculation.

In certain embodiments, a pharmaceutical composition described herein comprises about 1 mg to about 1.5 g, about 10 mg to about 1000 mg, or about 10 mg to about 200 mg of a steroidal compound (e.g., a testosterone alkyl ester, such as testosterone undecanoate). In specific embodiments, a pharmaceutical composition described herein comprises about 10 mg to about 50 mg, about 15 mg to about 40 mg, about 20 mg, to about 30 mg, or about 25 mg of steroidal compound (e.g., a testosterone alkyl ester, such as testosterone undecanoate). In other embodiments, a pharmaceutical composition described herein comprises about 70 mg to about 150 mg, about 80 mg to about 140 mg, about 90 mg to about 140 mg, about 100 mg to about 130 mg, about 110 mg to about 130 mg, about 110 mg to about 120 mg, about 130 mg to about 180 mg, about 180 mg to about 230 mg, about 230 mg to about 280 mg, about 280 mg to about 330 mg, about 330 mg to about 380 mg, about 380 mg to about 430 mg, about 430 mg to about 480 mg, about 480 mg to about 530 mg, or about 530 mg to about 580 mg of a steroidal compound (e.g., a testosterone alkyl ester, such as testosterone undecanoate). In some embodiments, a pharmaceutical composition described herein comprises about 0.1 mg to about 10 mg of a steroidal compound (e.g., a testosterone alkyl ester such as testosterone undecanoate) per kg of an individual to whom the oral dosage form is to be administered. In certain embodiments, a pharmaceutical composition described herein comprises an amount of a steroidal compound (e.g., a testosterone alkyl ester, such as testosterone undecanoate) sufficient to provide about 1 mg to about 1 g, about 5 mg to about 500 mg, about 10 mg to about 300 mg, or about 20 to about 250 mg of a steroidal compound (e.g., a testosterone alkyl ester, such as testosterone undecanoate) to an individual upon once a day, twice a day, three times a day, or four times a day oral administration.

In some embodiments, the amount of pharmaceutical agent administered to the subject in need of treatment is expressed as testosterone equivalents. For example, 1.58 mg of testosterone undecanoate is equivalent to 1 mg of testosterone, therefore 1.58 mg of testosterone undecanoate is 1 mg T equivalents. The corresponding T equivalents can be calculated according for other testosterone esters. Thus, in one embodiment, the amount of T equivalents administered to an individual in need of treatments is chosen from 1-10 mg, 10-40 mg, 40-60 mg, 60-80 mg, 80-100 mg, 100-125 mg, 125-150 mg, 150-200 mg, 200-250 mg, 250-300 mg, 300-350 mg, 350-400 mg, 400-500 mg, 500-600 mg, 600-700 mg, 700-800 mg, 800-900 mg, 900-1000 mg, 1000-1250 mg, 1250-1500 mg, 1500-2000 mg, 2000-2500 mg, 2500-3000 mg, 3000-4000 mg, 4000-5000 mg or more than 5000 mg.

Thus, in one embodiment, the amount of testosterone undecanoate administered to an individual in need of treatments is chosen from 1-10 mg, 10-40 mg, 40-60 mg, 60-80 mg, 80-100 mg, 100-125 mg, 125-150 mg, 150-200 mg, 200-250 mg, 250-300 mg, 300-350 mg, 350-400 mg, 400-500 mg, 500-600 mg, 600-700 mg, 700-800 mg, 800-900 mg, 900-1000 mg, 1000-1250 mg, 1250-1500 mg, 1500-2000 mg, 2000-2500 mg, 2500-3000 mg, 3000-4000 mg, 4000-5000 mg or more than 5000 mg.

Thus, in one embodiment, the amount of testosterone tridecanoate administered to an individual in need of treatments is chosen from 1-10 mg, 10-40 mg, 40-60 mg, 60-80 mg, 80-100 mg, 100-125 mg, 125-150 mg, 150-200 mg, 200-250 mg, 250-300 mg, 300-350 mg, 350-400 mg, 400-500 mg, 500-600 mg, 600-700 mg, 700-800 mg, 800-900 mg, 900-1000 mg, 1000-1250 mg, 1250-1500 mg, 1500-2000 mg, 2000-2500 mg, 2500-3000 mg, 3000-4000 mg, 4000-5000 mg or more than 5000 mg.

Thus, in one embodiment, the amount of testosterone dodecanoate administered to an individual in need of treatments is chosen from 1-10 mg, 10-40 mg, 40-60 mg, 60-80 mg, 80-100 mg, 100-125 mg, 125-150 mg, 150-200 mg, 200-250 mg, 250-300 mg, 300-350 mg, 350-400 mg, 400-500 mg, 500-600 mg, 600-700 mg, 700-800 mg, 800-900 mg, 900-1000 mg, 1000-1250 mg, 1250-1500 mg, 1500-2000 mg, 2000-2500 mg, 2500-3000 mg, 3000-4000 mg, 4000-5000 mg or more than 5000 mg.

The amount of pharmaceutical agent administered to an individual in need of treatment, e.g., having a viral infection like a respiratory virus (e.g., SARS-CoV-2) or a disease caused by a virus (e.g., COVID-19) depends on a number of factors, as understand by a person of ordinary skill in the art, like the route of administration and the nature of the active agent among other things.

In another aspect, the amount of androgen receptor agonist (e.g., a testosterone agent) administered to the individual in need of treatment is an amount sufficient to modulate the expression of one or more androgen receptor (e.g., a testosterone agent) target genes in one or more target tissues of the individual in need of treatment. In yet another aspect, the amount of androgen receptor agonist (e.g., a testosterone agent) administered to the individual in need of treatment is an amount sufficient to reduce one or more serum cytokines. In again another aspect, the amount of androgen receptor agonist (e.g., a testosterone agent) administered to the individual in need of treatment is an amount sufficient to slow the progression of lung disease. In one aspect, the amount of androgen receptor agonist (e.g., a testosterone agent) administered to the individual in need of treatment is an amount sufficient reduce the risk of death. In one aspect, the amount of androgen receptor agonist (e.g., a testosterone agent) administered to the individual in need of treatment is an amount sufficient reduce the risk of disease progression or slow disease progression.

In some embodiments, the amount of pharmaceutical agent (e.g., androgen receptor agonist or a testosterone agent) administered to an individual in need of treatment is an amount sufficient to modulate the level of one or more serum cytokines. In some embodiments, the amount of pharmaceutical agent administered to an individual in need of treatment is an amount sufficient to decrease the level of one or more serum cytokines. In some embodiments, the amount of pharmaceutical agent administered to an individual in need of treatment is an amount sufficient to reduce the rate of increase of the levels of one or more serum cytokines. In some aspects of these embodiments, the one or more serum cytokines is chosen from the list of cytokines and chemokines: Eotaxin, fibroblast growth factor (FGF), granulocyte-colony stimulating factor (G-CSF), interferon-γ (IFN-γ), interferon γ-induced protein (IP-10), interleukin-2 (IL-2), interleukin-4 (IL-4), interleukin-5 (IL-5), interleukin-6 (IL-6), interleukin-7 (IL-7), interleukin-8 (IL-8), interleukin-9 (IL9), interleukin-10 (IL-10), interleukin-12 (IL-12), interleukin-13 (IL-13), interleukin-15 (IL-15), interleukin-17 (IL-17), interleukin-1β (IL-1β), interleukin 1 receptor agonist (IL-1rα), monocyte chemoattractant protein-1 (MCP-1), platelet-derived growth factor-BB (PDGF-BB), regulated upon activation, normal T Cell expressed and presumably secreted chemokine (RANTES), tumor necrosis factor-α (TNF-α), and vascular endothelial growth factor (VEGF).

The compositions and dosage forms described herein can include a variety of pharmaceutically acceptable carriers known in the art. In another embodiment, the androgen receptor agonist (e.g., testosterone agent) can further comprise a lipophilic additive or a hydrophilic additive. In one aspect, the lipophilic additive can be a lipophilic surfactant. In one aspect, the androgen receptor agonist (e.g., testosterone agent) can comprise a testosterone ester and a lipophilic surfactant.

In some embodiments, the at least one pharmaceutically acceptable carrier is any carrier suitable for delivering an efficacious amount of a steroidal compound, e.g., a testosterone alkyl ester, to an individual. In some embodiments, the at least one pharmaceutically acceptable carrier is or comprises a hydrophilic carrier (e.g., a hydrophilic surfactant or hydrophilic additive). In certain embodiments, the at least one pharmaceutically acceptable carrier is a lipophilic carrier (e.g., a lipophilic surfactant or lipophilic additive). In some embodiments, the at least one pharmaceutically acceptable carrier is a hydrophilic carrier (e.g., a hydrophilic surfactant or hydrophilic additive) and a lipophilic carrier (e.g., a lipophilic surfactant or lipophilic additive). In certain embodiments, the hydrophilic carrier is a hydrophilic triglyceride. In specific embodiments, the hydrophilic triglyceride is a polyoxylated castor oil, or a polyoxylated hydrogenated castor oil. In some embodiments, any pharmaceutical composition provided herein consists essentially of a lipophilic carrier or combination of lipophilic carriers. In certain embodiments, any pharmaceutical composition provided herein comprises a lipophilic carrier and less than 10% w/w, less than 5% w/w or is substantially free of a hydrophilic carrier. In certain embodiments, any pharmaceutical composition provided herein comprises a lipophilic carrier and less than 10% w/w, less than 5% w/w or is substantially free of a hydrophilic carrier. In some embodiments, the pharmaceutical composition comprising a carrier (e.g., a hydrophilic carrier and/or a lipophilic carrier), the pharmaceutical composition is a gel, a jelly, a paste, or the like. In certain embodiments, e.g., wherein a pharmaceutical composition comprising a hydrophilic carrier and/or a lipophilic carrier, a viscosity enhancing agent or a solidifying agent is utilized to afford a pharmaceutical composition that is a gel, a jelly, a paste, or the like. Thus, in certain embodiments, the at least one pharmaceutically acceptable carrier is a hydrophilic carrier (e.g., a hydrophilic surfactant or hydrophilic additive) and a viscosity enhancing or solidifying agent. In certain embodiments, the at least one pharmaceutically acceptable carrier is a lipophilic carrier (e.g., a lipophilic surfactant or lipophilic additive) and a viscosity enhancing or solidifying agent. In some embodiments, the at least one pharmaceutically acceptable carrier is or comprises a hydrophilic carrier (e.g., a hydrophilic surfactant or hydrophilic additive), a lipophilic carrier (e.g., a lipophilic surfactant or lipophilic additive), and a viscosity enhancing or solidifying agent. In some embodiments, the at least one pharmaceutically acceptable carrier is or comprises an amphiphilic or zwitterionic carrier (e.g., an amphiphilic surfactant or amphiphilic additive). In certain embodiments, the pharmaceutically acceptable carrier is any carrier suitable for achieving one or more of the pharmacokinetic and/or pharmacodynamic profiles set forth herein.

In certain embodiments, the at least one pharmaceutically acceptable carrier comprises at least one hydrophilic carrier (e.g., hydrophilic surfactant). In some embodiments, the hydrophilic carrier is a polyoxylated glyceride (e.g., mono-, di-, or tri-glyceride), a polyoxylated vegetable oil, a polyoxylated hydrogenated vegetable oil, a polyoxylated fatty acid (mono-, or di-substituted), combinations thereof, or the like. In certain embodiments, the at least one pharmaceutically acceptable carrier comprises or further comprises a lipophilic carrier. Lipophilic carriers are selected from, by way of non-limiting example, a lipophilic surfactant, a vegetable oil (e.g., castor oil), a fatty acid, a fatty alcohol, a glyceride (e.g., mono-, di-, or tri-glyceride), a hydrogenated vegetable oil, a Vitamin E compound (e.g., d,l-.alpha.-tocopherol), a triglyceride, a fatty acid, polyoxylated fatty acid, polyoxylated triglyceride, polyoxylated vegetable oil, or combinations thereof. In some embodiments, polyoxylated compounds include polyethoxylated compounds.

In certain embodiments, the at least one hydrophilic carrier makes up about 1% to about 99% w/w, about 2% to about 80% w/w, about 2% to about 50% w/w, or about 10% to about 40% w/w of any pharmaceutical composition described herein. In some embodiments, lipophilic carriers make up about 1% w/w to about 99% w/w, about 2% to about 80% w/w, about 10% w/w to about 80% w/w, about 30% w/w, to about 80% w/w, or about 40% to about 80% w/w of any pharmaceutical composition described herein.

In addition to the androgenic agent, the pharmaceutical compositions and dosage forms can further include at least one additional pharmaceutically active agent or can be formulated to be co-administered with other active agents in order to treat a target condition. Non-limiting examples of additional active agents that can be included with or co-administered with the pharmaceutical composition or dosage form include: a vaccine, a protease inhibitor, RNA or DNA polymerase inhibitors, anti-viral agents, lung function improving agents, anti-diabetic agents, anti-inflammatory agents, anti IL-6 lowering agents, proton pump inhibitors, Immunomodulators, other sex hormones, plasma antibody treatment, the like, and combinations thereof. Such compounds could be co-administered with the compositions and dosage forms of the present invention in order to provide improved clinical outcomes.

In some examples, the active agent can be administered to a subject as a primary therapy, and administering the therapeutically effective amount of the androgen receptor agonist (e.g., testosterone agent) to the subject can be an adjunct therapy to the primary therapy. For example, an active agent such as an antiviral can be administered to the subject as a primary therapy in treating a virally-induced respiratory infection (e.g., .COVID-19) and the therapeutically effective amount of the androgen receptor agonist (e.g., testosterone agent) can be a secondary therapy in treating a virally-induced respiratory condition (e.g., COVID-19).

In some examples, the active agent can be administered to a subject as an adjunct therapy, and administering the therapeutically effective amount of the androgen receptor agonist (e.g., testosterone agent) to the subject can be a primary therapy to the adjunct therapy. For example, an active agent such as an antiviral can be administered to the subject as a secondary therapy in treating a virally-induced respiratory infection (e.g., .COVID-19) and the therapeutically effective amount of the androgen receptor agonist (e.g., testosterone agent) can be a primary therapy in treating a virally-induced respiratory condition (e.g., COVID-19).

In some examples, the additional active agent can also include a vaccine. Non-limiting examples of vaccines can include adenovirus vaccine, coronavirus, coxsackie B vaccine, cytomegalovirus vaccine, dengue vaccine, Eastern equine encephalitis vaccine, ebola vaccine, enterovirus vaccine, Epstein-barr vaccine, hepatitis A vaccine, hepatitis B vaccine, hepatitis C vaccine, hepatitis E vaccine, HIV vaccine, human papillomavirus vaccine, HTLV-1 T-lymphotrophic vaccine, influenza vaccine, Japanese encephalitis vaccine, Marburg vaccine, measles vaccine, mumps vaccine, norovirus vaccine, polio vaccine, rabies vaccine, respiratory syncytial virus (RSV) vaccine, rotavirus vaccine, rubella vaccine, severe acute respiratory syndrome (SARS) vaccine, varicella vaccine, smallpox vaccine, West Nile virus vaccine, yellow fever vaccine, anthrax vaccine, DPT vaccine, Q fever vaccine, Hib vaccine, tuberculosis vaccine, meningococcal vaccine, typhoid vaccine, pneumococcal vaccine, cholera vaccine, caries vaccine, ehrlichiosis vaccine, leprosy vaccine, lyme disease vaccine, Staphylococcus aureus vaccine, Streptococcus pyogenes vaccine, syphilis vaccine, tularemia vaccine, Yersinia pestis vaccine, the like, or combinations thereof.

In one embodiment, the additional active agent can also include any suitable protease inhibitor. Non-limiting examples of protease inhibitors can include atazanavir, darunavir, fosamprenavir, indinavir, lopinavir/ritonavir, nelfinavir, ritonavir, saquinavir, tipranavir, atazanavir/cobicistat, darunavir/cobicistat, the like, and combinations thereof. In one embodiment, depending on the specific viral infection being treated, the methods and combination described herein can be used in conjunction with an inhibitor of a protein involved in replicating or transcribing viral nucleic acids. For example, SARS-CoV-2 is known to have an RNA-dependent RNA polymerase (e.g., RdRp, also named nsp12). Thus, the methods and composition described herein can be used in conjunction with an inhibitor or modulator of this polymerase.

In one embodiment, the additional active agent can also include any suitable antiviral. Non-limiting examples of antivirals can include amantadine, rimantadine, ritonavir, cobicistat, peginterferon alfa-2a, peginterferon alfa 2b, maraviroc, raltegravir, dolutegravir, elvitegravir, sofosbuvir, enfuvirtide, fomivirsen, foscarnet, oseltamivir, zanamivir, peramivir, etravirine, efavirenz, nevirapine, delavirdine, rilpivirine, daclatasvir, adefovir, entecavir, telbivudine, didanosine, tenofovir, abacavir, lamivudine, zidovudine, stavudine, emtricitabine, zalcitabine, boceprevir, simeprevir, fosamprenavir, lopinavir, darunavir, telaprevir, ritonavir, tipranavir, atazanavir, nelfinavir, amprenavir, indinavir, saquinavir, ganciclovir, valacyclovir, famciclovir, acyclovir, valganciclovir, ribavirin, cidofovir, or the like; carbapenems such as doripenem, meropenem, cilastatin, ertapenem, the like, or combinations thereof.

In one embodiment, the additional active agent can also include any suitable lung-function enhancing agents. Non-limiting examples of lung-function enhancing agents can include anti-IgE, combination medications, inhaled steroids, leukotriene modifiers, oral steroids, theophylline, the like, and combinations thereof.

In one embodiment, the additional active agent can also include any suitable anti-diabetic agent. Non-limiting examples of anti-diabetic agents can include: metformin, sulfonylureas, meglitinides, thiazolidinediones, DPP-4 inhibitors, GLP-1 receptor agonists, SGLT2 inhibitors, insulin, the like, and combinations thereof.

In some examples, the additional active agent can also include any suitable anti-inflammatory agent. Non-limiting examples of anti-inflammatory agents can include ibuprofen, naproxen, aspirin, diclofenac, celecoxib, sulindac, oxaprozin, piroxicam, indomethacin, meloxicam, fenoprofen, difunisal, etodolac, ketorolac, meclofenamate, nabumetone, salsalate, ketoprofen, tolmetin, flurbiprofen, mefenamic acid, famotidine, bromfenac, nepafenac, prednisone, cortisone, hydrocortisone, methylprednisolone, deflazacort, prednisolone, fludrocortisone, amcinonide, betamethasone diproprionate, clobetasol, clocortolone, dexamethasone, diflorasone, durasteride, flumethasone pivalate, flunisolide, fluocinolone acetonide, fluocinonide, fluorometholone, fluticasone propionate, flurandrenolide, hydroflumethiazide, the like, hydrates thereof, acids thereof, bases thereof, or salts thereof, or combinations thereof.

In one embodiment, the additional active agent can also include any suitable anti-IL-6 agent. Non-limiting examples of anti-IL-6 agents can include: tocilizumab, siltuximab, sarilumab, the like, and combinations thereof.

In one embodiment, the additional active agent can also include any suitable proton pump inhibitors. Non-limiting examples of proton pump inhibitors can include: dexlansoprazole, esomeprazole, lansoprazole, omeprazole, pantoprazole, rabeprazole, the like, and combinations thereof.

In one embodiment, the additional active agent can also include any suitable immunomodulators. Non-limiting examples of immunomodulators can include: 6Mercaptopurine, 6MP, anakinra, BG-12, C1 esterase inhibitor recombinant, C1 inhibitor human, dimethyl fumarate, diroximel fumarate, ecallantide, emapalumab, emapalumab-lzsg, fingolimod, glatiramer, icatibant, Infergen, interferon alfa n3, interferon alfacon 1, interferon beta 1a, interferon beta 1b, mercaptopurine, monomethyl fumarate, peginterferon beta-1a, rilonacept, siltuximab, the like, and combinations thereof.

In one embodiment, the additional active agent can also include any suitable sex hormones. Non-limiting examples of sex hormones can include: progestogens, androgens, estrogens, and combinations thereof.

In one embodiment, the additional active agent can also include any suitable plasma antibody treatment, including convalescent plasma therapy.

### EXAMPLES

The following examples are provided to promote a more clear understanding of certain embodiments of the present invention, and are in no way meant as a limitation thereon.

### Example E2

In one example, the T/E2 ratio can be affected by topical and oral T (TLANDO) as depicted in Table E2.

| | | T (ng/dL) | E2 (pg/mL) | T/E2 Ratio | % CBL |
|---|---|---|---|---|---|
| TLANDO | Baseline | 244 | 18 | 128 | |
| | EOS | 541 | 24.7 | 206.8 | 61.5625% |
| Topical | Baseline | 251 | 18 | 132 | |
| | EOS | 463 | 27.7 | 157.8 | 19.54545% |

*CBL - change from baseline
*EOS is 52 weeks.
*TLANDO: 225 mg dose BID starting with titration (final dose range 150-300 mg BID).
*Topical: 40 mg QD starting with titration (final dose range 20-80 mg).
*The normal range for T/E in men is approximately 125-200 (unitless), but there is not a well-defined normal range. Men's T/E ratio appears to fluctuate with the seasons as well (highest in spring, lowest in fall).

### Pharmaceutical Compositions:

In some embodiments, the testosterone compositions disclosed herein can include a lipophilic additive which can include a lipophilic surfactant. As used herein a surfactant is considered to be a lipophilic surfactant when it has an HLB value of 10 or less. Various lipophilic surfactants can be used including, but not limited to mono-, di-glycerides of fatty acids like glyceryl monolinoleate (e.g. Maisine® 35-1), mono- and di glycerides of caprylic, capric acid (e.g. Capmul® MCM), glyceryl monooleate, reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (e.g. Labrafil® M 2125 CS), PEG-6 almond oil (e.g. Labrafil® M 1966 CS), PEG-6 apricot kernel oil (e.g. Labrafil® M 1944 CS), PEG-6 olive oil (e.g. Labrafil® M 1980 CS), PEG-6 peanut oil (e.g. Labrafil® M 1969 CS), PEG-6 hydrogenated palm kernel oil (e.g. Labrafil®. M 2130 BS), PEG-6 palm kernel oil (e.g. Labrafil® M 2130 CS), PEG-6 triolein (e.g. Labrafil® M 2735 CS), PEG-8 corn oil (e.g. Labrafil® WL 2609 BS), PEG-20 corn glycerides (e.g. Crovol® M40), PEG-20 almond glycerides (e.g. Crovol® A40), lipophilic polyoxyethylene-polyoxypropylene block co-polymers (e.g. Pluronic® L92, L101, L121 etc.); propylene glycol fatty acid esters, such as propylene glycol monolaurate (e.g. Lauroglycol FCC), propylene glycol ricinoleate (e.g. Propymuls), propylene glycol monooleate (e.g. Myverol P-O6), propylene glycol dicaprylate/dicaprate (e.g. Captex® 200), and propylene glycol dioctanoate (e.g. Captex® 800), propylene glycol mono-caprylate (e.g. Capryol® 90); propylene glycol oleate (e.g. Lutrol OP2000); propylene glycol myristate; propylene glycol mono stearate; propylene glycol hydroxy stearate; propylene glycol ricinoleate; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (e.g. Arlacel® 186); sterol and sterol derivatives such as cholesterol, sitosterol, phytosterol, phytosterol fatty acid esters, PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (e.g. Arlacel 20), sorbitan monopalmitate (e.g. Span-40), sorbitan monooleate (e.g. Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; fatty acids such as capric acid, caprylic acid, oleic acid, linoleic acid, myristic acid, menthol, menthol derivatives, lecithin, phosphatidyl choline, bile salts, and the like, and mixtures thereof. It is important to note that some lipophilic surfactants may also function as the solubilizer component of the compositions and oral dosage forms.

In one embodiment, the lipophilic surfactant can be selected from the group consisting of glyceryl monolinoleate (e.g. Maisine® 35-1), mono- and di glycerides of caprylic, capric acid (e.g. Capmul® MCM), glyceryl monooleate, propylene glycol mono caprylate, propylene glycol oleate, propylene glycol monostearate, propylene glycol monolaurate, propylene glycol mono-oleate, propylene glycol dicaprylate/dicaprate, sorbitan monooleate, PEG-5 hydrogenated castor oil, PEG-7 hydrogenated castor oil, PEG-9 hydrogenated castor oil, PEG-6 corn oil, PEG-6 almond oil, PEG-6 apricot kernel oil, PEG-6 olive oil, PEG-6 peanut oil, PEG-6 hydrogenated palm kernel oil, sorbitan monolaurate (e.g. Arlacel 20), sorbitan monopalmitate, sorbitan monooleate, sorbitan monostearate, sorbitan tristearate, sorbitan monolaurate, sorbitan monopalmitate, sorbitan monooleate, sorbitan trioleate, sorbitan sesquioleate, sorbitan tristearate, sorbitan monoisostearate, and combinations thereof. In some embodiments, the lipophilic surfactants can comprise at least about 10, 20, 30, 40, 50, 60, 70, 80, 90 or 95 wt % of the total pharmaceutically acceptable carrier. It should be noted that the combinations of two or more lipophilic surfactants from the same or different classes therein are also within the scope of this invention and are together can be referred to as the lipophilic surfactant, unless otherwise stated.

In some embodiments of the present invention, the oral pharmaceutical compositions or dosage forms (e.g. capsule or tablet) can include a hydrophilic additive. In one embodiment, hydrophilic additive is a selected from the group consisting of hydrophilic surfactant, celluloses—such as hydroxypropyl celluloses low molecular weight, low viscosity types (e.g. Methocel® E5, E6, E10 E15, LV100 etc. grades) and hydroxypropyl celluloses having higher molecular weight, medium to high viscosity (e.g. Methocel® K4M, K15M, K100M etc); polyvinylpyrrolidones (e.g. Kollidon k17, K30 etc); polyvinyl acetates and combinations thereof.

In one embodiment, the hydrophilic additive can be a hydrophilic surfactant. A surfactant is considered to be a hydrophilic surfactant when it has an HLB value of greater than 10. Non-limiting examples of hydrophilic surfactants include non-ionic surfactants, ionic surfactants and zwitterionic surfactants. Specifically the hydrophilic surfactants suitable for the current invention include, but not limited to alcohol-oil transesterification products; polyoxyethylene hydrogenated vegetable oils; polyoxyethylene vegetable oils; alkyl sulphate salts, dioctyl sulfosuccinate salts; polyethylene glycol fatty acids esters; polyethylene glycol fatty acids mono- and di-ester mixtures; polysorbates, polyethylene glycol derivatives of tocopherol and the like It should be noted that the combinations of two or more hydrophilic surfactants from the same or different classes are within the scope of this invention and are together can be referred to as the hydrophilic surfactant unless explicitly specified. In one embodiment, the hydrophilic additive can be a hydrophilic surfactant. Non-limiting examples of hydrophilic surfactants can include PEG-8 caprylic/capric glycerides, lauroyl macrogol-32 glyceride, stearoyl macrogol glyceride, PEG-40 hydrogenated castor oil, PEG-35 castor oil, sodium lauryl sulfate, sodium dioctyl sulfosuccinate, polyethylene glycol fatty acids mono- and di-ester mixtures, polysorbate 80, polysorbate 20, polyethylene glycol 1000 tocopherol succinate, phytosterols, phytosterol fatty acid esters, and mixtures thereof.

In some embodiments, surfactants utilized in the pharmaceutical compositions described herein include sterols and derivatives of sterols. In various embodiments, these surfactants are hydrophilic or lipophilic. Examples of hydrophilic sterol surfactants are lanosterol PEG-24 cholesterol ether (e.g. Solulan C-24, Amerchol), PEG-30 soya sterol (e.g. Nikkol BPS-30, from Nikko), PEG-25 phyto sterol (e.g. Nikkol BPSH-25 from Nikko), PEG-30 cholestanol (e.g. Nikkol DHC, from Nikko). Examples of Lipophilic Sterol Surfactants are Cholesterol, sitosterol, Phytosterol (e.g. GENEROL series from Henkel), PEG-5 soya sterol (e.g. Nikkol BPS-S, from Nikko), PEG-10 soya sterol (e.g. Nikkol BPS-10 from Nikko), PEG-20 soya sterol (e.g. Nikkol BPS-20 from Nikko).

### Compositional Examples

The drug formulation composition examples in the following Tables are provided to promote a more clear understanding of certain embodiments of the present invention, and are in no way meant as a limitation thereon.

TABLE 1
Drug + Carriers

| | Compositions (w/w %) | |
|---|---|---|
| | Testosterone ester or | |
| Composition | a combination thereof* | Carrier |
| A | 10-15 | 85-90 |
| B | 15-20 | 80-85 |
| C | 20-30 | 70-80 |
| D | 30-40 | 60-70 |
| E | 40-50 | 50-60 |

*Drug is not limited to ones in the table, but includes testosterone, dehydroepiandrosterone (DHEA), dehydroepiandrosterone sulfate (DHEA-S), androstenedione (A4), Androsterone, and androstenediol (A5), nandrolone, dimethandrolone, dihydrotestosterone, dihydrotestosterone esters or a combination thereof. It can be untreated (PS < 450 μm), sieved (PS < 450 μm), milled (PS < 150 μm), micronized (1 μm < PS < 25 μm), nanosized (PS < 1 μm), amorphous, or solubilized.

TABLE 2
Carrier Components

| | | Carrier No. | | | | | | | | | | | | | |
|---|---|---|---|---|---|---|---|---|---|---|---|---|---|---|---|
| Component | | I | II | III | IV | V | VI | VII | VIII | IX | X | XI | XII | XIII | XIV |
| Solubilizer | Propylene glycol | Yes | — | — | — | — | — | — | — | — | — | — | — | — | — |
| | mono or di-laurate | | | | | | | | | | | | | | |
| | Propylene glycol | — | Yes | — | — | — | — | — | — | — | — | — | — | — | — |
| | mono or di- | | | | | | | | | | | | | | |
| | caprylate | | | | | | | | | | | | | | |
| | Glyceryl mono or | — | — | Yes | — | — | — | — | — | — | — | — | — | — | — |
| | di-linoleate | | | | | | | | | | | | | | |
| | Glyceryl mono or | — | — | — | Yes | — | — | — | — | — | — | — | — | — | — |
| | di-oleate | | | | | | | | | | | | | | |
| | Glyceryl mono or | — | — | — | — | Yes | — | — | — | — | — | — | — | — | — |
| | di-stearate | | | | | | | | | | | | | | |
| | Glyceryl palmito- | — | — | — | — | — | Yes | — | — | — | — | — | — | — | — |
| | stearate | | | | | | | | | | | | | | |
| | Oleic acid | — | — | — | — | — | — | Yes | — | — | — | — | — | — | — |
| | Stearic acid | — | — | — | — | — | — | — | Yes | — | — | — | — | — | — |
| | Linoleic acid | — | — | — | — | — | — | — | — | Yes | — | — | — | — | — |
| | Peppermint oil | — | — | — | — | — | — | — | — | — | Yes | — | — | — | — |
| | Omega-3 | — | — | — | — | — | — | — | — | — | — | Yes | — | — | — |
| | EPA/DHA | | | | | | | | | | | | | | |
| | Vitamin E | — | — | — | — | — | — | — | — | — | — | — | Yes | — | — |
| | Alcohol | — | — | — | — | — | — | — | — | — | — | — | — | Yes | — |
| | Co-solvent* | — | — | — | — | — | — | — | — | — | — | — | — | — | Yes |
| Hydrophilic | Cremophor, | Yes | Yes | Yes | Yes | Yes | Yes | Yes | Yes | Yes | Yes | Yes | Yes | Yes | Yes |
| additive | Tween, SLS, | | | | | | | | | | | | | | |
| | Poloxamer, Co- | | | | | | | | | | | | | | |
| | polymer, and/or | | | | | | | | | | | | | | |
| | combinations | | | | | | | | | | | | | | |
| Other | Anti-oxidant, | q.s. | q.s. | q.s. | q.s. | q.s. | q.s. | q.s. | q.s. | q.s. | q.s. | q.s. | q.s. | q.s. | q.s. |
| additive | solidifier, flow | | | | | | | | | | | | | | |
| | agent, solvent, | | | | | | | | | | | | | | |
| | and/or | | | | | | | | | | | | | | |
| | combinations | | | | | | | | | | | | | | |

*Co-solvent can be a combination of 2 or more solubilizers that are listed in this table as well as include propylene glycol, polyethylene glycol, glycerol, sorbitol, DMA, and so on.

Composition A-I. Compositions composed of Drug API 10-15%, Propylene glycol mono or di-laurate, and additives.

| | | Compositions (w/w %) | | | | | | | | | | | |
|---|---|---|---|---|---|---|---|---|---|---|---|---|---|
| | | Propylene | | | | | | | | | | | |
| | | glycol | | | | | | | | | | | |
| Comp | | mono or | Hydrophilic additives* | | | | | | Other additives | | | | Ratio of |
| A-I | | di- | Cremophor | Tween- | | Poloxamer | Co- | Combina- | Anti- | Solidi- | Flow | Combina- | API: |
| No. | | laurate | RH 40** | 80*** | SLS | 407**** | polymer**** | tions ****** | oxidant | fier | agent | tions ******* | Solubilizer |
| a | i | 85-90 | — | — | — | | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

* Hydrophiic additives are not limited to ones listed in this table.
** Cremophor is not limited to Cremophor RH 40 but Cremphor EL and RH 35.
*** Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-I. Compositions composed of Drug API 15-20%, Propylene glycol mono or di-laurate, and additives

| | | Compositions (w/w %) | | | | | | | | | | | |
|---|---|---|---|---|---|---|---|---|---|---|---|---|---|
| | | Propylene | | | | | | | | | | | |
| | | glycol | Hydrophilic additives* | | | | | | | | | | |
| Comp. | | mono or | Cremo- | | | | Co- | | Other additives | | | | Ratio of |
| B-I | | di- | phor RH | Tween- | | Poloxa- | poly- | Combina- | Anti- | Solidi- | Flow | Combina- | API: |
| No. | | laurate | 40** | 80*** | SLS | mer407**** | mer***** | tions****** | oxidant | fier | agent | tions******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-I. Compositions composed of Drug API 20-30%, Propylene glycol mono or di-laurate, and additives

| | | Compositions (w/w %) | | | | | | | | | | | |
|---|---|---|---|---|---|---|---|---|---|---|---|---|---|
| | | Propylene | | | | | | | | | | | |
| | | glycol | Hydrophilic additives* | | | | | | | | | | |
| Comp. | | mono or | Cremo- | | | | Co- | | Other additives | | | | Ratio of |
| C-I | | di- | phor RH | Tween- | | Poloxa- | poly- | Combina- | Anti- | Solidi- | Flow | Combina- | API: |
| No. | | laurate | 40** | 80*** | SLS | mer407**** | mer***** | tions****** | oxidant | fier | agent | tions******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-I. Compositions composed of Drug API 30-40%, Propylene glycol mono or di-laurate, and additives

| | | Compositions (w/w %) | | | | | | | | | | | |
|---|---|---|---|---|---|---|---|---|---|---|---|---|---|
| | | Propylene | | | | | | | | | | | |
| | | glycol | Hydrophilic additives* | | | | | | | | | | |
| Comp. | | mono or | Cremo- | | | | Co- | | Other additives | | | | Ratio of |
| D-I | | di- | phor RH | Tween- | | Poloxa- | poly- | Combina- | Anti- | Solidi- | Flow | Combina- | API: |
| No. | | laurate | 40** | 80*** | SLS | mer407**** | mer***** | tions****** | oxidant | fier | agent | tions******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition E-I. Compositions composed of Drug API 40-50%, Propylene glycol mono or di-laurate, and additives

| | | Compositions (w/w %) | | | | | | | | | | | |
|---|---|---|---|---|---|---|---|---|---|---|---|---|---|
| | | Propylene | | | | | | | | | | | |
| | | glycol | Hydrophilic additives* | | | | | | | | | | |
| Comp. | | mono or | Cremo- | | | | Co- | | Other additives | | | | Ratio of |
| E-I | | di- | phor RH | Tween- | | Poloxa- | poly- | Combina- | Anti- | Solidi- | Flow | Combina- | API: |
| No. | | laurate | 40** | 80*** | SLS | mer407**** | mer***** | tions****** | oxidant | fier | agent | tions******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:0.6-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

*Hydrophilic additives are not limited to ones listed in this table.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-II. Compositions composed of Drug API 10-15%, Propylene glycol mono or di-caprylate, and additives

| | | Compositions (w/w %) | | | | | | | | | | | |
|---|---|---|---|---|---|---|---|---|---|---|---|---|---|
| | | Propylene | | | | | | | | | | | |
| | | glycol | Hydrophilic additives* | | | | | | | | | | |
| Comp. | | mono or | Cremo- | | | | Co- | | Other additives | | | | Ratio of |
| A-II | | di- | phor RH | Tween- | | Poloxa- | poly- | Combina- | Anti- | Solidi- | Flow | Combina- | API: |
| No. | | caprylate | 40** | 80*** | SLS | mer407**** | mer***** | tions****** | oxidant | fier | agent | tions******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-II. Compositions composed of Drug API 15-20%, Propylene glycol mono or di-caprylate, and additives

| | | Compositions (w/w %) | | | | | | | | | | | |
|---|---|---|---|---|---|---|---|---|---|---|---|---|---|
| | | Propylene | | | | | | | | | | | |
| | | glycol | Hydrophilic additives* | | | | | | | | | | |
| Comp. | | mono or | Cremo- | | | | Co- | | Other additives | | | | Ratio of |
| B-II | | di- | phor RH | Tween- | | Poloxa- | poly- | Combina- | Anti- | Solidi- | Flow | Combina- | API: |
| No. | | caprylate | 40** | 80*** | SLS | mer407**** | mer***** | tions****** | oxidant | fier | agent | tions******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-II. Compositions composed of Drug API 20-30%, Propylene glycol mono or di-caprylate, and additives

| | | Compositions (w/w %) | | | | | | | | | | | |
|---|---|---|---|---|---|---|---|---|---|---|---|---|---|
| | | Propylene | | | | | | | | | | | |
| | | glycol | Hydrophilic additives* | | | | | | | | | | |
| Comp. | | mono or | Cremo- | | | | Co- | | Other additives | | | | Ratio of |
| C-II | | di- | phor RH | Tween- | | Poloxa- | poly- | Combina- | Anti- | Solidi- | Flow | Combina- | API: |
| No. | | caprylate | 40** | 80*** | SLS | mer407**** | mer***** | tions****** | oxidant | fier | agent | tions******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-II. Compositions composed of Drug API 30-40%, Propylene glycol mono or di-caprylate, and additives

| | | Compositions (w/w %) | | | | | | | | | | | |
|---|---|---|---|---|---|---|---|---|---|---|---|---|---|
| | | Propylene | | | | | | | | | | | |
| | | glycol | Hydrophilic additives* | | | | | | | | | | |
| Comp. | | mono or | Cremo- | | | | Co- | | Other additives | | | | Ratio of |
| D-II | | di- | phor RH | Tween- | | Poloxa- | poly- | Combina- | Anti- | Solidi- | Flow | Combina- | API: |
| No. | | caprylate | 40** | 80*** | SLS | mer407**** | mer***** | tions****** | oxidant | fier | agent | tions******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition E-II. Compositions composed of Drug API 40-50%, Propylene glycol mono or di-caprylate, and additives

| | | Compositions (w/w %) | | | | | | | | | | | |
|---|---|---|---|---|---|---|---|---|---|---|---|---|---|
| | | Propylene | | | | | | | | | | | |
| | | glycol | Hydrophilic additives* | | | | | | | | | | |
| Comp. | | mono or | Cremo- | | | | Co- | | Other additives | | | | Ratio of |
| E-II | | di- | phor RH | Tween- | | Poloxa- | poly- | Combina- | Anti- | Solidi- | Flow | Combina- | API: |
| No. | | caprylate | 40** | 80*** | SLS | mer407**** | mer***** | tions****** | oxidant | fier | agent | tions******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:0.6-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-III. Compositions composed of Drug API 10-15%, Glyceryl mono or di-linoleate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono or | Cremo- | | | | Co- | | Other additives | | | | Ratio of |
| A-III | | di- | phor RH | Tween- | | Poloxa- | poly- | Combina- | Anti- | Solidi- | Flow | Combina- | API: |
| No. | | linoleate | 40** | 80*** | SLS | mer407**** | mer***** | tions****** | oxidant | fier | agent | tions******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-III. Compositions composed of Drug API 15-20%, Glyceryl mono or di-linoleate, and additives

| | | Compositions (w/w %) | | | | | | | | | | | |
|---|---|---|---|---|---|---|---|---|---|---|---|---|---|
| | | Glycerol | Hydrophilic additives* | | | | | | | | | | |
| Comp. | | mono or | Cremo- | | | | Co- | | Other additives | | | | Ratio of |
| B-III | | di- | phor RH | Tween- | | Poloxa- | poly- | Combina- | Anti- | Solidi- | Flow | Combina- | API: |
| No. | | linoleate | 40** | 80*** | SLS | mer407**** | mer***** | tions****** | oxidant | fier | agent | tions******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-III. Compositions composed of Drug API 20-30%, Glyceryl mono or di-linoleate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono or | Cremo- | | | | Co- | | Other additives | | | | Ratio of |
| C-III | | di- | phor RH | Tween- | | Poloxa- | poly- | Combina- | Anti- | Solidi- | Flow | Combina- | API: |
| No. | | linoleate | 40** | 80*** | SLS | mer407**** | mer***** | tions****** | oxidant | fier | agent | tions******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-III. Compositions composed of Drug API 30-40%, Glyceryl mono or di-linoleate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono or | Cremo- | | | | Co- | | Other additives | | | | Ratio of |
| D-III | | di- | phor RH | Tween- | | Poloxa- | poly- | Combina- | Anti- | Solidi- | Flow | Combina- | API: |
| No. | | linoleate | 40** | 80*** | SLS | mer407**** | mer***** | tions****** | oxidant | fier | agent | tions******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition E-III. Compositions composed of Drug API 40-50%, Glyceryl mono or di-linoleate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| E-III | | or di- | RH | Tween- | | Poloxa- | poly- | Combi- | Anti- | Solidi- | Flow | Combi- | API: |
| No. | | linoleate | 40** | 80*** | SLS | mer407**** | mer***** | nations****** | oxidant | fier | agent | nations******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:0.6-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-IV. Compositions composed of Drug API 10-15%, Glyceryl mono or di-oleate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| A-IV | | or di- | RH | Tween- | | Poloxa- | poly- | Combi- | Anti- | Solidi- | Flow | Combi- | API: |
| No. | | oleate | 40** | 80*** | SLS | mer407**** | mer***** | nations****** | oxidant | fier | agent | nations******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-30 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-IV. Compositions composed of Drug API 15-20%, Glyceryl mono or di-oleate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| B-IV | | or di- | RH | Tween- | | Poloxa- | poly- | Combi- | Anti- | Solidi- | Flow | Combi- | API: |
| No. | | oleate | 40** | 80*** | SLS | mer407**** | mer***** | nations****** | oxidant | fier | agent | nations******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-IV. Compositions composed of Drug API 20-30%, Glyceryl mono or di-oleate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| C-IV | | or di- | RH | Tween- | | Poloxa- | poly- | Combi- | Anti- | Solidi- | Flow | Combi- | API: |
| No. | | oleate | 40** | 80*** | SLS | mer407**** | mer***** | nations****** | oxidant | fier | agent | nations******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-IV. Compositions composed of Drug API 30-40%, Glyceryl mono or di-oleate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| D-IV | | or di- | RH | Tween- | | Poloxa- | poly- | Combi- | Anti- | Solidi- | Flow | Combi- | API: |
| No. | | oleate | 40** | 80*** | SLS | mer407**** | mer***** | nations****** | oxidant | fier | agent | nations******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition E-IV. Compositions composed of Drug API 40-50%, Glyceryl mono or di-oleate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| E-IV | | or di- | RH | Tween- | | Poloxa- | poly- | Combi- | Anti- | Solidi- | Flow | Combi- | API: |
| No. | | oleate | 40** | 80*** | SLS | mer407**** | mer***** | nations****** | oxidant | fier | agent | nations******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:06-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:06-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:06-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:06-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:06-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:06-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-V. Compositions composed of Drug API 10-15%, Glyceryl mono or di-stearate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| A-V | | or di- | RH | Tween- | | Poloxa- | poly- | Combi- | Anti- | Solidi- | Flow | Combi- | API: |
| No. | | stearate | 40** | 80*** | SLS | mer407**** | mer***** | nations****** | oxidant | fier | agent | nations******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-V. Compositions composed of Drug API 15-20%, Glyceryl mono or di-stearate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| B-V | | or di- | RH | Tween- | | Poloxa- | poly- | Combi- | Anti- | Solidi- | Flow | Combi- | API: |
| No. | | stearate | 40** | 80*** | SLS | mer407**** | mer***** | nations****** | oxidant | fier | agent | nations******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-V. Compositions composed of Drug API 20-30%, Glyceryl mono or di-stearate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| C-V | | or di- | RH | Tween- | | Poloxa- | poly- | Combi- | Anti- | Solidi- | Flow | Combi- | API: |
| No. | | stearate | 40** | 80*** | SLS | mer407**** | mer***** | nations****** | oxidant | fier | agent | nations******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-V. Compositions composed of Drug API 30-40%, Glyceryl mono or di-stearate, and additives

| | | Compositions (w/w %) | | | | | | | | | | | |
|---|---|---|---|---|---|---|---|---|---|---|---|---|---|
| | | Glyceryl | Hydrophilic additives* | | | | | | | | | | |
| Comp. | | mono | Cremophor | | | | Co- | | Other additives | | | | Ratio of |
| D-V | | or di- | RH | Tween- | | Poloxa- | poly- | Combi- | Anti- | Solidi- | Flow | Combi- | API: |
| No. | | stearate | 40** | 80*** | SLS | mer407**** | mer***** | nations****** | oxidant | fier | agent | nations******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-VI. Compositions composed of Drug API 10-15%, Glyceryl palmito-stearate, and additives

| | | Compositions (w/w %) | | | | | | | | | | | |
|---|---|---|---|---|---|---|---|---|---|---|---|---|---|
| | | | Hydrophilic additives* | | | | | | | | | | |
| Comp. | | Glyceryl | Cremophor | | | | Co- | | Other additives | | | | Ratio of |
| A-VI | | palmito- | RH | Tween- | | Poloxa- | poly- | Combi- | Anti- | Solidi- | Flow | Combi- | API: |
| No. | | stearate | 40** | 80*** | SLS | mer407**** | mer***** | nations****** | oxidant | fier | agent | nations******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 20-30 | 0-1 | 0-10 | 0-5 | 0-15 | ~1:4.4 |
| | vi | 50-90 | — | — | — | — | — | 0-30 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-VI. Compositions composed of Drug API 15-20%, Glyceryl palmito-stearate, and additives

| | | Compositions (w/w %) | | | | | | | | | | | |
|---|---|---|---|---|---|---|---|---|---|---|---|---|---|
| | | | Hydrophilic additives* | | | | | | | | | | |
| Comp. | | Glyceryl | Cremophor | | | | Co- | | Other additives | | | | Ratio of |
| B-VI | | palmito- | RH | Tween- | | Poloxa- | poly- | Combi- | Anti- | Solidi- | Flow | Combi- | API: |
| No. | | stearate | 40** | 80*** | SLS | mer407**** | mer***** | nations****** | oxidant | fier | agent | nations******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-VI. Compositions composed of Drug API 20-30%, Glyceryl palmito-stearate, and additives

| | | Compositions (w/w %) | | | | | | | | | | | |
|---|---|---|---|---|---|---|---|---|---|---|---|---|---|
| | | | Hydrophilic additives* | | | | | | | | | | |
| Comp. | | Glyceryl | Cremophor | | | | Co- | | Other additives | | | | Ratio of |
| C-VI | | palmito- | RH | Tween- | | Poloxa- | poly- | Combi- | Anti- | Solidi- | Flow | Combi- | API: |
| No. | | stearate | 40** | 80*** | SLS | mer407**** | mer***** | nations****** | oxidant | fier | agent | nations******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-VI. Compositions composed of Drug API 30-40%, Glyceryl palmito-stearate, and additives

| | | Compositions (w/w %) | | | | | | | | | | | |
|---|---|---|---|---|---|---|---|---|---|---|---|---|---|
| | | | Hydrophilic additives* | | | | | | | | | | |
| Comp. | | Glyceryl | Cremophor | | | Polox- | Co- | | Other additives | | | | Ratio of |
| D-VI | | palmito- | RH | Tween- | | amer | poly- | Combi- | Anti- | Solidi- | Flow | Combi- | API: |
| No. | | stearate | 40** | 80*** | SLS | 407**** | mer***** | nations****** | oxidant | fier | agent | nations******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~-1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones isted in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition E-VI. Compositions composed of Drug API 40-50%, Glyceryl palmito-stearate, and additives

| | | Compositions (w/w %) | | | | | | | | | | | |
|---|---|---|---|---|---|---|---|---|---|---|---|---|---|
| | | | Hydrophilic additives* | | | | | | | | | | |
| Comp. | | Glyceryl | Cremophor | | | Polox- | Co- | | Other additives | | | | Ratio of |
| E-VI | | palmito- | RH | Tween- | | amer | poly- | Combi- | Anti- | Solidi- | Flow | Combi- | API: |
| No. | | stearate | 40** | 80*** | SLS | 407**** | mer***** | nations****** | oxidant | fier | agent | nations******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:06-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:06-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:06-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:06-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:06-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:06-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-VII. Compositions composed of Drug API 10-15%, Oleic acid, and additives

| | | Compositions (w/w %) | | | | | | | | | | | |
|---|---|---|---|---|---|---|---|---|---|---|---|---|---|
| Comp. | | | Hydrophilic additives* | | | | | | | | | | |
| A- | | | Cremo- | | | | Co- | | Other additives | | | | Ratio of |
| VII | | Oleic | phor RH | Tween- | | Poloxa- | poly- | Combi- | Anti- | Solidi- | Flow | Combi- | API: |
| No. | | acid | 40** | 80*** | SLS | mer407**** | mer***** | nations****** | oxidant | fier | agent | nations******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | iv | 50-60 | — | — | — | — | — | 25-35 | 0-1 | 0-10 | 0-5 | 0-15 | ~1:4.4 |
| | v | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-VII. Compositions composed of Drug API 15-20%, Oleic acid, and additives

| | | Compositions (w/w %) | | | | | | | | | | | |
|---|---|---|---|---|---|---|---|---|---|---|---|---|---|
| Comp. | | | Hydrophilic additives* | | | | | | | | | | |
| B- | | | | | | | Co- | | Other additives | | | | Ratio of |
| VII | | Oleic | Cremophor | Tween- | | Poloxa- | poly- | Combi- | Anti- | Solidi- | Flow | Combi- | API: |
| No. | | acid | RH 40** | 80*** | SLS | mer407**** | mer***** | nations****** | oxidant | fier | agent | nations******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-VII. Compositions composed of Drug API 20-30%, Oleic acid, and additives

| | | Compositions (w/w %) | | | | | | | | | | | |
|---|---|---|---|---|---|---|---|---|---|---|---|---|---|
| | | | Hydrophilic additives* | | | | | | | | | | |
| Comp. | | | Cremophor | | | | Co- | | Other additives | | | | Ratio of |
| C-VII | | Oleic | RH | Tween- | | Poloxa- | poly- | Combi- | Anti- | Solidi- | Flow | Combi- | API: |
| No. | | acid | 40** | 80*** | SLS | mer407**** | mer***** | nations****** | oxidant | fier | agent | nations******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-VII. Compositions composed of Drug API 30-40%, Oleic acid, and additives

Compositions (w/w %)
Hydrophilic additives*

| Comp. | | Cremophor | | | | Co- | | Other additives | | | | Ratio of |
|---|---|---|---|---|---|---|---|---|---|---|---|---|
| D-VII | Oleic | RH | Tween- | | Poloxa- | poly- | Combi- | Anti- | Solidi- | Flow | Combi- | API: |
| No. | acid | RH 40** | 80*** | SLS | mer407**** | mer***** | nations****** | oxidant | fier | agent | nations******* | Solubilizer |

| | | | | | | | | | | | | | |
|---|---|---|---|---|---|---|---|---|---|---|---|---|---|
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition E-VII. Compositions composed of Drug API 40-50%, Oleic acid, and additives

| | | Compositions (w/w %) | | | | | | | | | | | |
|---|---|---|---|---|---|---|---|---|---|---|---|---|---|
| | | | Hydrophilic additives* | | | | | | | | | | |
| Comp. | | | Cremophor | | | | Co- | | Other additives | | | | Ratio of |
| E-VII | | Oleic | RH | Tween- | | Poloxa- | poly- | Combi- | Anti- | Solidi- | Flow | Combi- | API: |
| No. | | acid | 40** | 80*** | SLS | mer407**** | mer***** | nations****** | oxidant | fier | agent | nations******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:0.6-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-VIII. Compositions composed of Drug API 10-15%, Stearic acid, and additives

| | | Compositions (w/w %) | | | | | | | | | | | |
|---|---|---|---|---|---|---|---|---|---|---|---|---|---|
| | | | Hydrophilic additives* | | | | | | | | | | |
| Comp. | | | Cremophor | | | | Co- | | Other additives | | | | Ratio of |
| A-VIII | | Stearic | RH | Tween- | | Poloxa- | poly- | Combi- | Anti- | Solidi- | Flow | Combi- | API: |
| No. | | acid | 40** | 80*** | SLS | mer407**** | mer***** | nations****** | oxidant | fier | agent | nations******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-VIII. Compositions composed of Drug API 15-20%, Stearic acid, and additives

| | | Compositions (w/w %) | | | | | | | | | | | |
|---|---|---|---|---|---|---|---|---|---|---|---|---|---|
| Comp. | | | Hydrophilic additives* | | | | | | | | | | |
| B- | | | Cremophor | | | | Co- | | Other additives | | | | Ratio of |
| VIII | | Stearic | RH | Tween- | | Poloxa- | poly- | Combi- | Anti- | Solidi- | Flow | Combi- | API: |
| No. | | acid | 40** | 80*** | SLS | mer407**** | mer***** | nations****** | oxidant | fier | agent | nations******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-VIII. Compositions composed of Drug API 20-30%, Stearic acid, and additives

| | | Compositions (w/w %) | | | | | | | | | | | |
|---|---|---|---|---|---|---|---|---|---|---|---|---|---|
| Comp. | | | Hydrophilic additives* | | | | | | | | | | |
| C- | | | Cremophor | | | | Co- | | Other additives | | | | Ratio of |
| VIII | | Stearic | RH | Tween- | | Poloxa- | poly- | Combi- | Anti- | Solidi- | Flow | Combi- | API: |
| No. | | acid | 40** | 80*** | SLS | mer407**** | mer***** | nations****** | oxidant | fier | agent | nations******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophlic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-VIII. Compositions composed of Drug API 30-40%, Stearic acid, and additives

| | | Compositions (w/w %) | | | | | | | | | | | |
|---|---|---|---|---|---|---|---|---|---|---|---|---|---|
| Comp. | | | Hydrophilic additives* | | | | | | | | | | |
| D- | | | Cremophor | | | | Co- | | Other additives | | | | Ratio of |
| VIII | | Stearic | RH | Tween- | | Poloxa- | poly- | Combi- | Anti- | Solidi- | Flow | Combi- | API: |
| No. | | acid | 40** | 80*** | SLS | mer407**** | mer***** | nations****** | oxidant | fier | agent | nations******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition E-VIII. Compositions composed of Drug API 40-50%, Stearic acid, and additives

| | | Compositions (w/w %) | | | | | | | | | | | |
|---|---|---|---|---|---|---|---|---|---|---|---|---|---|
| Comp. | | | Hydrophilic additives* | | | | | | | | | | |
| E- | | | Cremophor | | | | Co- | | Other additives | | | | Ratio of |
| VIII | | Stearic | RH | Tween- | | Poloxa- | poly- | Combi- | Anti- | Solidi- | Flow | Combi- | API: |
| No. | | acid | 40** | 80*** | SLS | mer407**** | mer***** | nations****** | oxidant | fier | agent | nations******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:0.6-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-IX. Compositions composed of Drug API 10-15%, Linoleic acid, and

| | | Compositions (w/w %) | | | | | | | | | | | |
|---|---|---|---|---|---|---|---|---|---|---|---|---|---|
| Comp. | | | Hydrophilic additives* | | | | | | | | | | |
| A- | | | Cremophor | | | | Co- | | Other additives | | | | Ratio of |
| IX | | Linoleic | RH | Tween- | | Poloxa- | poly- | Combi- | Anti- | Solidi- | Flow | Combi- | API: |
| No. | | acid | 40** | 80*** | SLS | mer407**** | mer***** | nations****** | oxidant | fier | agent | nations******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-IX. Compositions composed of Drug API 15-20%, Linoleic acid, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| B-IX No. | | Linoleic acid | Cremophor RH 40** | Tween 80*** | SLS | Poloxamer 407**** | Copolymer***** | Combinations****** | Antioxidant | Solidifier | Flow agent | Combinations******* | API:Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | — | 0-1 | 0-10 | 0-5 | 0-15 | ~1:4.4 |
| | ii | 75-80 | — | — | — | — | — | — | 0-1 | 0-10 | 0-5 | 0-15 | ~1:4.1 |
| | iii | 70-75 | — | — | — | — | — | — | 0-1 | 0-10 | 0-5 | 0-15 | ~1:3.8 |
| | iv | 60-70 | — | — | — | — | — | — | 0-1 | 0-10 | 0-5 | 0-15 | ~1:3.4 |
| | v | 50-60 | — | — | — | — | — | — | 0-1 | 0-10 | 0-5 | 0-15 | ~1:2.9 |
| | vi | 50-90 | — | — | — | — | — | — | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as included others recited herein.
*******Combinations of other additives can be 2 or more other additives that are listed in this table as well as include colorant, coating agent, sweetener, and so on.

Composition C-IX. Compositions composed of Drug API 20-30%, Linoleic acid, and additives

| | | Compositions (w/w %) | | | | | | | | | | | |
|---|---|---|---|---|---|---|---|---|---|---|---|---|---|
| Comp. | | | Hydrophilic additives | | | | | | Other additives | | | | Ratio of |
| C-IX No. | | Linoleic acid | Chremophor RH 40** | Tween 80*** | SLS | Poloxamer 407**** | Copolymer***** | Combinations****** | Antioxidant | Solidifier | Flow agent | Combinations******* | API:Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition D-IX. Compositions composed of Drug API 30-40%, Linoleic acid, and additives

| | | Compositions (w/w %) | | | | | | | | | | | |
|---|---|---|---|---|---|---|---|---|---|---|---|---|---|
| Comp. | | | Hydrophilic additives | | | | | | Other additives | | | | Ratio of |
| D-IX No. | | Linoleic acid | Chremophor RH 40** | Tween 80*** | SLS | Poloxamer 407**** | Copolymer***** | Combinations****** | Antioxidant | Solidifier | Flow agent | Combinations******* | API:Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition E-IX. Compositions composed of Drug API 40-50%, Linoleic acid, and additives

| | | Compositions (w/w %) | | | | | | | | | | | |
|---|---|---|---|---|---|---|---|---|---|---|---|---|---|
| Comp. | | | Hydrophilic additives | | | | | | Other additives | | | | Ratio of |
| E-IX No. | | Linoleic acid | Chremophor RH 40** | Tween 80*** | SLS | Poloxamer 407**** | Copolymer***** | Combinations****** | Antioxidant | Solidifier | Flow agent | Combinations******* | API:Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:0.6-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition A-X. Compositions composed of Drug API 10-15%, Peppermint oil, and additives

| | | Compositions (w/w %) | | | | | | | | | | | |
|---|---|---|---|---|---|---|---|---|---|---|---|---|---|
| Comp. | | | Hydrophilic additives | | | | | | Other additives | | | | Ratio of |
| A-X No. | | Peppermint oil | Chremophor RH 40** | Tween 80*** | SLS | Poloxamer 407**** | Copolymer***** | Combinations****** | Antioxidant | Solidifier | Flow agent | Combinations******* | API:Solubilizer |
| a | i | 85-90 | — | — | — | — | — | — | 0-1 | 0-10 | 0-5 | 0-15 | ~1:1.2 |
| b | i | 80-85 | 0-4.5 | — | — | — | — | — | 0-1 | 0-10 | 0-5 | 0-15 | ~1:1.3 |
| | ii | 75-80 | 5-10 | — | — | — | — | — | 0-1 | 0-10 | 0-5 | 0-15 | ~1:1.2 |
| | iii | 70-75 | 10-15 | — | — | — | — | — | 0-1 | 0-10 | 0-5 | 0-15 | ~1:1.1 |
| | iv | 60-70 | 15-25 | — | — | — | — | — | 0-1 | 0-10 | 0-5 | 0-15 | ~1:0.9 |
| | v | 50-60 | 25-35 | — | — | — | — | — | 0-1 | 0-10 | 0-5 | 0-15 | ~1:0.8 |
| | vi | 50-90 | 0-35 | — | — | — | — | — | 0-1 | 0-10 | 0-5 | 0-15 | ~1:0.6-~1:1.5 |
| c | i | 80-85 | — | 0-4.5 | — | — | — | — | 0-1 | 0-10 | 0-5 | 0-15 | ~1:1.3 |
| | ii | 75-80 | — | 5-10 | — | — | — | — | 0-1 | 0-10 | 0-5 | 0-15 | ~1:1.2 |
| | iii | 70-75 | — | 10-15 | — | — | — | — | 0-1 | 0-10 | 0-5 | 0-15 | ~1:1.1 |
| | iv | 60-70 | — | 15-25 | — | — | — | — | 0-1 | 0-10 | 0-5 | 0-15 | ~1:0.9 |
| | v | 50-60 | — | 25-35 | — | — | — | — | 0-1 | 0-10 | 0-5 | 0-15 | ~1:0.8 |
| | vi | 50-90 | — | 0-35 | — | — | — | — | 0-1 | 0-10 | 0-5 | 0-15 | ~1:0.6-~1:1.5 |
| d | i | 80-85 | — | — | 0-4.5 | — | — | — | 0-1 | 0-10 | 0-5 | 0-15 | ~1:1.3 |
| | ii | 75-80 | — | — | 5-10 | — | — | — | 0-1 | 0-10 | 0-5 | 0-15 | ~1:1.2 |
| | iii | 70-75 | — | — | 10-15 | — | — | — | 0-1 | 0-10 | 0-5 | 0-15 | ~1:1.1 |
| | iv | 60-70 | — | — | 15-25 | — | — | — | 0-1 | 0-10 | 0-5 | 0-15 | ~1:0.9 |
| | v | 50-60 | — | — | 25-35 | — | — | — | 0-1 | 0-10 | 0-5 | 0-15 | ~1:0.8 |
| | vi | 50-90 | — | — | 0-35 | — | — | — | 0-1 | 0-10 | 0-5 | 0-15 | ~1:0.6-~1:1.5 |
| e | i | 80-85 | — | — | — | 0-4.5 | — | — | 0-1 | 0-10 | 0-5 | 0-15 | ~1:1.3 |
| | ii | 75-80 | — | — | — | 5-10 | — | — | 0-1 | 0-10 | 0-5 | 0-15 | ~1:1.2 |
| | iii | 70-75 | — | — | — | 10-15 | — | — | 0-1 | 0-10 | 0-5 | 0-15 | ~1:1.1 |
| | iv | 60-70 | — | — | — | 15-25 | — | — | 0-1 | 0-10 | 0-5 | 0-15 | ~1:0.9 |
| | v | 50-60 | — | — | — | 25-35 | — | — | 0-1 | 0-10 | 0-5 | 0-15 | ~1:0.8 |
| | vi | 50-90 | — | — | — | 0-35 | — | — | 0-1 | 0-10 | 0-5 | 0-15 | ~1:0.6-~1:1.5 |
| f | i | 80-85 | — | — | — | — | 0-4.5 | — | 0-1 | 0-10 | 0-5 | 0-15 | ~1:1.3 |
| | ii | 75-80 | — | — | — | — | 5-10 | — | 0-1 | 0-10 | 0-5 | 0-15 | ~1:1.2 |
| | iii | 70-75 | — | — | — | — | 10-15 | — | 0-1 | 0-10 | 0-5 | 0-15 | ~1:1.1 |
| | iv | 60-70 | — | — | — | — | 15-25 | — | 0-1 | 0-10 | 0-5 | 0-15 | ~1:0.9 |
| | v | 50-60 | — | — | — | — | 25-35 | — | 0-1 | 0-10 | 0-5 | 0-15 | ~1:0.8 |
| | vi | 50-90 | — | — | — | — | 0-35 | — | 0-1 | 0-10 | 0-5 | 0-15 | ~1:0.6-~1:1.5 |
| g | i | 80-85 | — | — | — | — | — | 0-4.5 | 0-1 | 0-10 | 0-5 | 0-15 | ~1:1.3 |
| | ii | 75-80 | — | — | — | — | — | 5-10 | 0-1 | 0-10 | 0-5 | 0-15 | ~1:1.2 |
| | iii | 70-75 | — | — | — | — | — | 10-15 | 0-1 | 0-10 | 0-5 | 0-15 | ~1:1.1 |
| | iv | 60-70 | — | — | — | — | — | 15-25 | 0-1 | 0-10 | 0-5 | 0-15 | ~1:0.9 |
| | v | 50-60 | — | — | — | — | — | 25-35 | 0-1 | 0-10 | 0-5 | 0-15 | ~1:0.8 |
| | vi | 50-90 | — | — | — | — | — | 0-35 | 0-1 | 0-10 | 0-5 | 0-15 | ~1:0.6-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition B-X. Compositions composed of Drug API 15-20%, Peppermint oil, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| B-X No. | | Peppermint oil | Cremophor RH 40** | Tween 80*** | SLS | Poloxamer 407**** | Copolymer***** | Combinations****** | Antioxidant | Solidifier | Flow agent | Combinations****** | API:Solubilizer |
| a | i | 85-90 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:1:5 |

*Hydrophilic additives are not limited to ones listed in this table but include others recited herein.
**Cremophor is not limited to Cremophor RH 40 but Cremophor EL and RH 35.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition C-X. Compositions composed of Drug API 20-30%, Peppermint oil, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| C-X No. | | Peppermint oil | Cremophor RH 40** | Tween 80*** | API:Solubilizer | Poloxamer 407**** | Copolymer***** | Combinations****** | Antioxidant | Solidifier | Flow agent | Combinations****** | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition D-X. Compositions composed of Drug API 30-40%, Peppermint oil, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| D-X No. | | Peppermint oil | Cremophor RH 40** | Tween 80*** | SLS | Poloxamer 407**** | Copolymer***** | Combinations****** | Antioxidant | Solidifier | Flow agent | Combinations****** | API:Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition E-X. Compositions composed of Drug API 40-50%, Peppermint oil, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| E-X No. | | Peppemint oil | Cremophor RH 40** | Tween 80*** | SLS | Poloxamer**** | Copolymer***** | Combinations****** | Antioxidant | Solidifier | Flow agent | Combinations****** | API:Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.6- |
| | vi | 30-60 | — | — | — | 0-30 | — | — | 0-1 | 0-10 | 0-5 | 0-15 | ~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

*Hydrophilic additives are not limited to ones listed in this table but include others recited herein.
**Cremophor is not limited to Cremophor RH 40 but Cremophor EL and RH 35.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition A-XI. Compositions composed of Drug API 10-15%, Omega-3 EPA/DHA, and additives

| | | Compositions (w/w %) | | | | | | | | | | | |
|---|---|---|---|---|---|---|---|---|---|---|---|---|---|
| Comp. | | Omega-3 | Hydrophilic additives* | | | | | | Other additives | | | | Ratio of |
| A-XI No. | | EPA/DHA | Cremophor RH 40** | Tween 80*** | SLS | Poloxamer**** | Copolymer***** | Combinations****** | Antioxidant | Solidifier | Flow agent | Combinations****** | API:Solubilizer |
| a | i | 85-90 | — | — | — | — | — | — | 0-1 | 0-10 | 0-5 | 0-15 | ~1:1.8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition B-XI. Compositions composed of Drug API 15-20%, Omega-3 EPA/DHA, and additives

| | | Compositions (w/w %) | | | | | | | | | | | |
|---|---|---|---|---|---|---|---|---|---|---|---|---|---|
| Comp. | | Omega-3 | Hydrophilic additives* | | | | | | Other additives | | | | Ratio of |
| B-XI No. | | EPA/DHA | Cremophor RH 40** | Tween 80*** | SLS | Poloxamer**** | Copolymer***** | Combinations****** | Antioxidant | Solidifier | Flow agent | Combinations****** | API:Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition C-XI. Compositions composed of Drug API 20-30%, Omega-3 EPA/DHA, and additives

| | | Compositions (w/w %) | | | | | | | | | | | |
|---|---|---|---|---|---|---|---|---|---|---|---|---|---|
| Comp. | | Omega-3 | Hydrophilic additives* | | | | | | Other additives | | | | Ratio of |
| C-XI No. | | EPA/DHA | Cremophor RH 40** | Tween 80*** | SLS | Poloxamer 407**** | Copolymer***** | Combinations****** | Antioxidant | Solidifier | Flow agent | Combinations****** | API:Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition D-XI. Compositions composed of Drug API 30-40%, Omega-3 EPA/DHA, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| D-XI No. | | Omega-3 EPA/DHA | Cremophor RH 40** | Tween 80*** | SLS | Poloxamer 407**** | Copolymer***** | Combinations****** | Antioxidant | Solidifier | Flow agent | Combinations****** | API:Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
****Poloxamer is not limited to Poloxamer 407 but Poloxamer 12, 188, and 335.
*****Co-polymer can be PVP, PMC, or other compunds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetner, and so on.

Composition E-XI. Compositions composed of Drug API 40-50%, Omega-3 EPA/DHA, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| E-XI | | Omega-3 | Cremophor | Tween - | | Poloxamer | Co-poly- | Combina- | Anti- | | Flow | Combina- | API: |
| No. | | EPA/DHA | RH 40** | 80*** | SLS | 407**** | mer***** | tions****** | oxidant | Solidifier | agent | tions******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:06-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:06-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:06-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:06-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:06-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:06-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-XII. Compositions composed of Drug API 10-15%, Vitamin E, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| A-XII | | Vitamin | Cremophor | Tween - | | Poloxamer | Co-poly- | Combina- | Anti- | | Flow | Combina- | API: |
| No. | | E | RH 40** | 80*** | SLS | 407**** | mer***** | tions****** | oxidant | Solidifier | agent | tions******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-XII. Compositions composed of Drug API 15-20%, Vitamin E, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| B-XII | | Vitamin | Cremophor | Tween - | | Poloxamer | Co-poly- | Combina- | Anti- | | Flow | Combina- | API: |
| No. | | E | RH 40** | 80*** | SLS | 407**** | mer***** | tions****** | oxidant | Solidifier | agent | tions******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 80-85 | — | — | — | 0-4.5 | — | — | 0-1 | 0-10 | 0-5 | 0-15 | ~1:4.4 |
| | ii | 75-80 | — | — | — | 5-10 | — | — | 0-1 | 0-10 | 0-5 | 0-15 | ~1:4.1 |
| | iii | 70-75 | — | — | — | 10-20 | — | — | 0-1 | 0-10 | 0-5 | 0-15 | ~1:3.8 |
| | iv | 60-70 | — | — | — | 20-30 | — | — | 0-1 | 0-10 | 0-5 | 0-15 | ~1:3.4 |
| | v | 50-60 | — | — | — | 30-40 | — | — | 0-1 | 0-10 | 0-5 | 0-15 | ~1:2.9 |
| | vi | 50-90 | — | — | — | 0-40 | — | — | 0-1 | 0-10 | 0-5 | 0-15 | ~1:2.5-~1:5 |
| f | i | 80-85 | — | — | — | — | 0-4.5 | — | 0-1 | 0-10 | 0-5 | 0-15 | ~1:4.4 |
| | ii | 75-80 | — | — | — | — | 5-10 | — | 0-1 | 0-10 | 0-5 | 0-15 | ~1:4.1 |
| | iii | 70-75 | — | — | — | — | 10-20 | — | 0-1 | 0-10 | 0-5 | 0-15 | ~1:3.8 |
| | iv | 60-70 | — | — | — | — | 20-30 | — | 0-1 | 0-10 | 0-5 | 0-15 | ~1:3.4 |
| | v | 50-60 | — | — | — | — | 30-40 | — | 0-1 | 0-10 | 0-5 | 0-15 | ~1:2.9 |
| | vi | 50-90 | — | — | — | — | 0-40 | — | 0-1 | 0-10 | 0-5 | 0-15 | ~1:2.5-~1:5 |
| g | i | 80-85 | — | — | — | — | — | 0-4.5 | 0-1 | 0-10 | 0-5 | 0-15 | ~1:4.4 |
| | ii | 75-80 | — | — | — | — | — | 5-10 | 0-1 | 0-10 | 0-5 | 0-15 | ~1:4.1 |
| | iii | 70-75 | — | — | — | — | — | 10-20 | 0-1 | 0-10 | 0-5 | 0-15 | ~1:3.8 |
| | iv | 60-70 | — | — | — | — | — | 20-30 | 0-1 | 0-10 | 0-5 | 0-15 | ~1:3.4 |
| | v | 50-60 | — | — | — | — | — | 30-40 | 0-1 | 0-10 | 0-5 | 0-15 | ~1:2.9 |
| | vi | 50-90 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-XII. Compositions composed of Drug API 20-30%, Vitamin E, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| C-XII | | Vitamin | Cremophor | Tween - | | Poloxamer | Co-poly- | Combina- | Anti- | | Flow | Combina- | API: |
| No. | | E | RH 40** | 80*** | SLS | 407**** | mer***** | tions****** | oxidant | Solidifier | agent | tions******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-60 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-XII. Compositions composed of Drug API 30-40%, Vitamin E, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| D-XII | | Vitamin | Cremophor | Tween - | | Poloxamer | Co-poly- | Combina- | Anti- | | Flow | Combina- | API: |
| No. | | E | RH 40** | 80*** | SLS | 407**** | mer***** | tions****** | oxidant | Solidifier | agent | tions******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 70-75 | — | 0-4.5 | — | — | — | — | 0-1 | 0-10 | 0-5 | 0-15 | ~1:1.9 |
| | ii | 65-70 | — | 5-10 | — | — | — | — | 0-1 | 0-10 | 0-5 | 0-15 | ~1:1.8 |
| | iii | 60-65 | — | 10-15 | — | — | — | — | 0-1 | 0-10 | 0-5 | 0-15 | ~1:1.6 |
| | iv | 50-60 | — | 15-25 | — | — | — | — | 0-1 | 0-10 | 0-5 | 0-15 | ~1:1.4 |
| | v | 40-50 | — | 25-35 | — | — | — | — | 0-1 | 0-10 | 0-5 | 0-15 | ~1:1.1 |
| | vi | 40-80 | — | 0-35 | — | — | — | — | 0-1 | 0-10 | 0-5 | 0-15 | ~1:1-~1:2 |
| d | i | 70-75 | — | — | 0-4.5 | — | — | — | 0-1 | 0-10 | 0-5 | 0-15 | ~1:1.9 |
| | ii | 65-70 | — | — | 5-10 | — | — | — | 0-1 | 0-10 | 0-5 | 0-15 | ~1:1.8 |
| | iii | 60-65 | — | — | 10-15 | — | — | — | 0-1 | 0-10 | 0-5 | 0-15 | ~1:1.6 |
| | iv | 50-60 | — | — | 15-25 | — | — | — | 0-1 | 0-10 | 0-5 | 0-15 | ~1:1.4 |
| | v | 40-50 | — | — | 25-35 | — | — | — | 0-1 | 0-10 | 0-5 | 0-15 | ~1:1.1 |
| | vi | 40-80 | — | — | 0-35 | — | — | — | 0-1 | 0-10 | 0-5 | 0-15 | ~1:1-~1:2 |
| e | i | 70-75 | — | — | — | 0-4.5 | — | — | 0-1 | 0-10 | 0-5 | 0-15 | ~1:1.9 |
| | ii | 65-70 | — | — | — | 5-10 | — | — | 0-1 | 0-10 | 0-5 | 0-15 | ~1:1.8 |
| | iii | 60-65 | — | — | — | 10-15 | — | — | 0-1 | 0-10 | 0-5 | 0-15 | ~1:1.6 |
| | iv | 50-60 | — | — | — | 15-25 | — | — | 0-1 | 0-10 | 0-5 | 0-15 | ~1:1.4 |
| | v | 40-50 | — | — | — | 25-35 | — | — | 0-1 | 0-10 | 0-5 | 0-15 | ~1:1.1 |
| | vi | 40-80 | — | — | — | 0-35 | — | — | 0-1 | 0-10 | 0-5 | 0-15 | ~1:1-~1:2 |
| f | i | 70-75 | — | — | — | — | 0-4.5 | — | 0-1 | 0-10 | 0-5 | 0-15 | ~1:1.9 |
| | ii | 65-70 | — | — | — | — | 5-10 | — | 0-1 | 0-10 | 0-5 | 0-15 | ~1:1.8 |
| | iii | 60-65 | — | — | — | — | 10-15 | — | 0-1 | 0-10 | 0-5 | 0-15 | ~1:1.6 |
| | iv | 50-60 | — | — | — | — | 15-25 | — | 0-1 | 0-10 | 0-5 | 0-15 | ~1:1.4 |
| | v | 40-50 | — | — | — | — | 25-35 | — | 0-1 | 0-10 | 0-5 | 0-15 | ~1:1.1 |
| | vi | 40-80 | — | — | — | — | 0-35 | — | 0-1 | 0-10 | 0-5 | 0-15 | ~1:1-~1:2 |
| g | i | 70-75 | — | — | — | — | — | 0-4.5 | 0-1 | 0-10 | 0-5 | 0-15 | ~1:1.9 |
| | ii | 65-70 | — | — | — | — | — | 5-10 | 0-1 | 0-10 | 0-5 | 0-15 | ~1:1.8 |
| | iii | 60-65 | — | — | — | — | — | 10-15 | 0-1 | 0-10 | 0-5 | 0-15 | ~1:1.6 |
| | iv | 50-60 | — | — | — | — | — | 15-25 | 0-1 | 0-10 | 0-5 | 0-15 | ~1:1.4 |
| | v | 40-50 | — | — | — | — | — | 25-35 | 0-1 | 0-10 | 0-5 | 0-15 | ~1:1.1 |
| | vi | 40-60 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition E-XII. Compositions composed of Drug API 40-50%, Vitamin E, and additives

| | | Compositions (w/w %) | | | | | | | | | | | |
|---|---|---|---|---|---|---|---|---|---|---|---|---|---|
| Comp. | | | Hydrophilic additives* | | | | | | Other additives | | | | Ratio of |
| E-XII | | Vitamin | Cremophor | Tween - | | Poloxamer | Co-poly- | Combina- | Anti- | | Flow | Combina- | API: |
| No. | | E | RH 40** | 80*** | SLS | 407**** | mer***** | tions****** | oxidant | Solidifier | agent | tions******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:0.6-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition A-XIII. Compositions composed of Drug API 10-15%, Alcohol, and additives

| | | Compositions (w/w %) | | | | | | | | | | | |
|---|---|---|---|---|---|---|---|---|---|---|---|---|---|
| | | Alcohol | | | | | | | | | | | |
| | | (e.g. ethyl | | | | | | | | | | | |
| | | alcohol, | | | | | | | | | | | |
| Comp. | | benzyl | Hydrophilic additives* | | | | | | Other additives | | | | Ratio of |
| A-XIII | | alcohol, | Cremophor | Tween - | | Poloxamer | Co-poly- | Combina- | Anti- | | Flow | Combina- | API: |
| No. | | etc) | RH 40** | 80*** | SLS | 407**** | mer***** | tions****** | oxidant | Solidifier | agent | tions******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition B-XIII Compositions composed of Drug API 15-20%, Alcohol, and additives

| | | Compositions (w/w %) | | | | | | | | | | | |
|---|---|---|---|---|---|---|---|---|---|---|---|---|---|
| | | Alcohol | | | | | | | | | | | |
| | | (e.g. ethyl | | | | | | | | | | | |
| Comp. | | alcohol, | | | | | | | | | | | |
| B- | | benzyl | Hydrophilic additives* | | | | | | Other additives | | | | Ratio of |
| XIII | | alcohol, | Cremophor | Tween - | | Poloxamer | Co-poly- | Combina- | Anti- | | Flow | Combina- | API: |
| No. | | etc) | RH 40** | 80*** | SLS | 407**** | mer***** | tions****** | oxidant | Solidifier | agent | tions******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 75-80 | — | — | — | — | — | 0-4.5 | 0-1 | 0-10 | 0-5 | 0-15 | ~1:4.4 |
| | ii | 70-75 | — | — | — | — | — | 5-10 | 0-1 | 0-10 | 0-5 | 0-15 | ~1:4.1 |
| | iii | 65-70 | — | — | — | — | — | 10-20 | 0-1 | 0-10 | 0-5 | 0-15 | ~1:3.8 |
| | iv | 55-65 | — | — | — | — | — | 20-30 | 0-1 | 0-10 | 0-5 | 0-15 | ~1:3.4 |
| | v | 45-55 | — | — | — | — | — | 30-40 | 0-1 | 0-10 | 0-5 | 0-15 | ~1:2.9 |
| | vi | 45-85 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition C-XIII Compositions composed of Drug API 20-30%, Alcohol, and additives

| | | Compositions (w/w %) | | | | | | | | | | | |
|---|---|---|---|---|---|---|---|---|---|---|---|---|---|
| | | Alcohol | | | | | | | | | | | |
| | | (e.g. ethyl | | | | | | | | | | | |
| Comp. | | alcohol, | | | | | | | | | | | |
| C- | | benzyl | Hydrophilic additives* | | | | | | Other additives | | | | Ratio of |
| XIII | | alcohol, | Cremophor | Tween - | | Poloxamer | Co-poly- | Combina- | Anti- | | Flow | Combina- | API: |
| No. | | etc) | RH 40** | 80*** | SLS | 407**** | mer***** | tions****** | oxidant | Solidifier | agent | tions******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-XIII. Compositions composed of Drug API 30-40%, Alcohol, and additives

| | | Compositions (w/w %) | | | | | | | | | | | |
|---|---|---|---|---|---|---|---|---|---|---|---|---|---|
| | | Alcohol | | | | | | | | | | | |
| | | (e.g. ethyl | | | | | | | | | | | |
| | | alcohol, | | | | | | | | | | | |
| Comp. | | benzyl | Hydrophilic additives* | | | | | | Other additives | | | | Ratio of |
| D-XIII | | alcohol, | Cremophor | Tween - | | Poloxamer | Co-poly- | Combina- | Anti- | | Flow | Combina- | API: |
| No. | | etc) | RH 40** | 80*** | SLS | 407**** | mer***** | tions****** | oxidant | Solidifier | agent | tions******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1:2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1:2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1:2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1:2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1:2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1:2 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.

Composition E-XIII. Compositions composed of Drug API 40-50%, Alcohol, and additives

| | | Compositions (w/w %) | | | | | | | | | | | |
|---|---|---|---|---|---|---|---|---|---|---|---|---|---|
| | | Alcohol | | | | | | | | | | | |
| | | (e.g. ethyl | | | | | | | | | | | |
| Comp. | | alcohol, | Hydrophilic additives* | | | | | | Other additives | | | | Ratio of |
| E-XIII | | benzyl | Cremophor | Tween - | | Poloxamer | Co-poly- | Combina- | Anti- | | Flow | Combina- | API: |
| No. | | alcohol) | RH 40** | 80*** | SLS | 407**** | mer***** | tions****** | oxidant | Solidifier | agent | tions******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:0.6-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.

Composition A-XIV. Compositions composed of Drug API 10-15%, Combinations of solubilizers, and additives

| | | Compositions (w/w %) | | | | | | | | | | | |
|---|---|---|---|---|---|---|---|---|---|---|---|---|---|
| | | Combina- | | | | | | | | | | | |
| | | tion of | | | | | | | | | | | |
| | | solubili- | | | | | | | | | | | |
| | | zers† (e.g. | | | | | | | | | | | |
| | | oleic acid | | | | | | | | | | | |
| | | and GDS, | | | | | | | | | | | |
| | | maisine | | | | | | | | | | | |
| Comp. | | 35-1 and | Hydrophilic additives* | | | | | | Other additives | | | | Ratio of |
| A-XIV | | capmul, | Cremophor | Tween - | | Poloxamer | Co-poly- | Combina- | Anti- | | Flow | Combina- | API: |
| No. | | etc) | RH 40** | 80*** | SLS | 407**** | mer***** | tions****** | oxidant | Solidifier | agent | tions******* | Solubilizer |
| a | i | 85-90 | — | — | — | — | — | — | 0-1 | 0-10 | 0-5 | 0-15 | ~1:8 |
| b | i | 80-85 | 0-4.5 | — | — | — | — | — | 0-1 | 0-10 | 0-5 | 0-15 | ~1:6.6 |
| | ii | 75-80 | 5-10 | — | — | — | — | — | 0-1 | 0-10 | 0-5 | 0-15 | ~1:6.2 |
| | iii | 70-75 | 10-15 | — | — | — | — | — | 0-1 | 0-10 | 0-5 | 0-15 | ~1:5.8 |
| | iv | 60-70 | 15-25 | — | — | — | — | — | 0-1 | 0-10 | 0-5 | 0-15 | ~1:5.2 |
| | v | 50-60 | 25-35 | — | — | — | — | — | 0-1 | 0-10 | 0-5 | 0-15 | ~1:4.4 |
| | vi | 50-90 | 0-35 | — | — | — | — | — | 0-1 | 0-10 | 0-5 | 0-15 | ~1:4-~1:8 |
| c | i | 80-85 | — | 0-4.5 | — | — | — | — | 0-1 | 0-10 | 0-5 | 0-15 | ~1:6.6 |
| | ii | 75-80 | — | 5-10 | — | — | — | — | 0-1 | 0-10 | 0-5 | 0-15 | ~1:6.2 |
| | iii | 70-75 | — | 10-15 | — | — | — | — | 0-1 | 0-10 | 0-5 | 0-15 | ~1:5.8 |
| | iv | 60-70 | — | 15-25 | — | — | — | — | 0-1 | 0-10 | 0-5 | 0-15 | ~1:5.2 |
| | v | 50-60 | — | 25-35 | — | — | — | — | 0-1 | 0-10 | 0-5 | 0-15 | ~1:4.4 |
| | vi | 50-90 | — | 0-35 | — | — | — | — | 0-1 | 0-10 | 0-5 | 0-15 | ~1:4-~1:8 |
| d | i | 80-85 | — | — | 0-4.5 | — | — | — | 0-1 | 0-10 | 0-5 | 0-15 | ~1:6.6 |
| | ii | 75-80 | — | — | 5-10 | — | — | — | 0-1 | 0-10 | 0-5 | 0-15 | ~1:6.2 |
| | iii | 70-75 | — | — | 10-15 | — | — | — | 0-1 | 0-10 | 0-5 | 0-15 | ~1:5.8 |
| | iv | 60-70 | — | — | 15-25 | — | — | — | 0-1 | 0-10 | 0-5 | 0-15 | ~1:5.2 |
| | v | 50-60 | — | — | 25-35 | — | — | — | 0-1 | 0-10 | 0-5 | 0-15 | ~1:4.4 |
| | vi | 50-90 | — | — | 0-35 | — | — | — | 0-1 | 0-10 | 0-5 | 0-15 | ~1:4-~1:8 |
| e | i | 80-85 | — | — | — | 0-4.5 | — | — | 0-1 | 0-10 | 0-5 | 0-15 | ~1:6.6 |
| | ii | 75-80 | — | — | — | 5-10 | — | — | 0-1 | 0-10 | 0-5 | 0-15 | ~1:6.2 |
| | iii | 70-75 | — | — | — | 10-15 | — | — | 0-1 | 0-10 | 0-5 | 0-15 | ~1:5.8 |
| | iv | 60-70 | — | — | — | 15-25 | — | — | 0-1 | 0-10 | 0-5 | 0-15 | ~1:5.2 |
| | v | 50-60 | — | — | — | 25-35 | — | — | 0-1 | 0-10 | 0-5 | 0-15 | ~1:4.4 |
| | vi | 50-90 | — | — | — | 0-35 | — | — | 0-1 | 0-10 | 0-5 | 0-15 | ~1:4-~1:8 |
| f | i | 80-85 | — | — | — | — | 0-4.5 | — | 0-1 | 0-10 | 0-5 | 0-15 | ~1:6.6 |
| | ii | 75-80 | — | — | — | — | 5-10 | — | 0-1 | 0-10 | 0-5 | 0-15 | ~1:6.2 |
| | iii | 70-75 | — | — | — | — | 10-15 | — | 0-1 | 0-10 | 0-5 | 0-15 | ~1:5.8 |
| | iv | 60-70 | — | — | — | — | 15-25 | — | 0-1 | 0-10 | 0-5 | 0-15 | ~1:5.2 |
| | v | 50-60 | — | — | — | — | 25-35 | — | 0-1 | 0-10 | 0-5 | 0-15 | ~1:4.4 |
| | vi | 50-90 | — | — | — | — | 0-35 | — | 0-1 | 0-10 | 0-5 | 0-15 | ~1:4-~1:8 |
| g | i | 80-85 | — | — | — | — | — | 0-4.5 | 0-1 | 0-10 | 0-5 | 0-15 | ~1:6.6 |
| | ii | 75-80 | — | — | — | — | — | 5-10 | 0-1 | 0-10 | 0-5 | 0-15 | ~1:6.2 |
| | iii | 70-75 | — | — | — | — | — | 10-15 | 0-1 | 0-10 | 0-5 | 0-15 | ~1:5.8 |
| | iv | 60-70 | — | — | — | — | — | 15-25 | 0-1 | 0-10 | 0-5 | 0-15 | ~1:5.2 |
| | v | 50-60 | — | — | — | — | — | 25-35 | 0-1 | 0-10 | 0-5 | 0-15 | ~1:4.4 |
| | vi | 50-90 | — | — | — | — | — | 0-35 | 0-1 | 0-10 | 0-5 | 0-15 | ~1:4-~1:8 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.

Composition B-XIV. Compositions composed of Drug API 15-20%, Combinations of solubilizers, and additives

| | | Compositions (w/w %) | | | | | | | | | | | |
|---|---|---|---|---|---|---|---|---|---|---|---|---|---|
| | | Combina- | | | | | | | | | | | |
| | | tion of | | | | | | | | | | | |
| | | solubili- | | | | | | | | | | | |
| | | zers† (e.g. | | | | | | | | | | | |
| | | oleic acid | | | | | | | | | | | |
| | | and GDS, | | | | | | | | | | | |
| | | maisine | | | | | | | | | | | |
| Comp. | | 35-1 and | Hydrophilic additives* | | | | | | Other additives | | | | Ratio of |
| B-XIV | | capmul, | Cremophor | Tween - | | Poloxamer | Co-poly- | Combina- | Anti- | | Flow | Combina- | API: |
| No. | | etc) | RH 40** | 80*** | SLS | 407**** | mer***** | tions****** | oxidant | Solidifier | agent | tions******* | Solubilizer |
| a | i | 80-85 | — | — | — | — | — | — | 0-1 | 0-10 | 0-5 | 0-15 | ~1:4.7 |
| b | i | 75-80 | 0-4.5 | — | — | — | — | — | 0-1 | 0-10 | 0-5 | 0-15 | ~1:4.4 |
| | ii | 70-75 | 5-10 | — | — | — | — | — | 0-1 | 0-10 | 0-5 | 0-15 | ~1:4.1 |
| | iii | 65-70 | 10-20 | — | — | — | — | — | 0-1 | 0-10 | 0-5 | 0-15 | ~1:3.8 |
| | iv | 55-65 | 20-30 | — | — | — | — | — | 0-1 | 0-10 | 0-5 | 0-15 | ~1:3.4 |
| | v | 45-55 | 30-40 | — | — | — | — | — | 0-1 | 0-10 | 0-5 | 0-15 | ~1:2.9 |
| | vi | 45-85 | 0-40 | — | — | — | — | — | 0-1 | 0-10 | 0-5 | 0-15 | ~1:2.5-~1:5 |
| c | i | 75-80 | — | 0-4.5 | — | — | — | — | 0-1 | 0-10 | 0-5 | 0-15 | ~1:4.4 |
| | ii | 70-75 | — | 5-10 | — | — | — | — | 0-1 | 0-10 | 0-5 | 0-15 | ~1:4.1 |
| | iii | 65-70 | — | 10-20 | — | — | — | — | 0-1 | 0-10 | 0-5 | 0-15 | ~1:3.8 |
| | iv | 55-65 | — | 20-30 | — | — | — | — | 0-1 | 0-10 | 0-5 | 0-15 | ~1:3.4 |
| | v | 45-55 | — | 30-40 | — | — | — | — | 0-1 | 0-10 | 0-5 | 0-15 | ~1:2.9 |
| | vi | 45-85 | — | 0-40 | — | — | — | — | 0-1 | 0-10 | 0-5 | 0-15 | ~1:2.5-~1:5 |
| d | i | 75-80 | — | — | 0-4.5 | — | — | — | 0-1 | 0-10 | 0-5 | 0-15 | ~1:4.4 |
| | ii | 70-75 | — | — | 5-10 | — | — | — | 0-1 | 0-10 | 0-5 | 0-15 | ~1:4.1 |
| | iii | 65-70 | — | — | 10-20 | — | — | — | 0-1 | 0-10 | 0-5 | 0-15 | ~1:3.8 |
| | iv | 55-65 | — | — | 20-30 | — | — | — | 0-1 | 0-10 | 0-5 | 0-15 | ~1:3.4 |
| | v | 45-55 | — | — | 30-40 | — | — | — | 0-1 | 0-10 | 0-5 | 0-15 | ~1:2.9 |
| | vi | 45-85 | — | — | 0-40 | — | — | — | 0-1 | 0-10 | 0-5 | 0-15 | ~1:2.5-~1:5 |
| e | i | 75-80 | — | — | — | 0-4.5 | — | — | 0-1 | 0-10 | 0-5 | 0-15 | ~1:4.4 |
| | ii | 70-75 | — | — | — | 5-10 | — | — | 0-1 | 0-10 | 0-5 | 0-15 | ~1:4.1 |
| | iii | 65-70 | — | — | — | 10-20 | — | — | 0-1 | 0-10 | 0-5 | 0-15 | ~1:3.8 |
| | iv | 55-65 | — | — | — | 20-30 | — | — | 0-1 | 0-10 | 0-5 | 0-15 | ~1:3.4 |
| | v | 45-55 | — | — | — | 30-40 | — | — | 0-1 | 0-10 | 0-5 | 0-15 | ~1:2.9 |
| | vi | 45-85 | — | — | — | 0-40 | — | — | 0-1 | 0-10 | 0-5 | 0-15 | ~1:2.5-~1:5 |
| f | i | 75-80 | — | — | — | — | 0-4.5 | — | 0-1 | 0-10 | 0-5 | 0-15 | ~1:4.4 |
| | ii | 70-75 | — | — | — | — | 5-10 | — | 0-1 | 0-10 | 0-5 | 0-15 | ~1:4.1 |
| | iii | 65-70 | — | — | — | — | 10-20 | — | 0-1 | 0-10 | 0-5 | 0-15 | ~1:3.8 |
| | iv | 55-65 | — | — | — | — | 20-30 | — | 0-1 | 0-10 | 0-5 | 0-15 | ~1:3.4 |
| | v | 45-55 | — | — | — | — | 30-40 | — | 0-1 | 0-10 | 0-5 | 0-15 | ~1:2.9 |
| | vi | 45-85 | — | — | — | — | 0-40 | — | 0-1 | 0-10 | 0-5 | 0-15 | ~1:2.5-~1:5 |
| g | i | 75-80 | — | — | — | — | — | 0-4.5 | 0-1 | 0-10 | 0-5 | 0-15 | ~1:4.4 |
| | ii | 70-75 | — | — | — | — | — | 5-10 | 0-1 | 0-10 | 0-5 | 0-15 | ~1:4.1 |
| | iii | 65-70 | — | — | — | — | — | 10-20 | 0-1 | 0-10 | 0-5 | 0-15 | ~1:3.8 |
| | iv | 55-65 | — | — | — | — | — | 20-30 | 0-1 | 0-10 | 0-5 | 0-15 | ~1:3.4 |
| | v | 45-55 | — | — | — | — | — | 30-40 | 0-1 | 0-10 | 0-5 | 0-15 | ~1:2.9 |
| | vi | 45-85 | — | — | — | — | — | 0-40 | 0-1 | 0-10 | 0-5 | 0-15 | ~1:2.5-~1:5 |

*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.

Composition C-XIV. Compositions composed of Drug API 20-30%, Combinations of solubilizers, and additives

| | | Compositions (w/w %) | | | | | | | | | | | |
|---|---|---|---|---|---|---|---|---|---|---|---|---|---|
| | | Combina- | | | | | | | | | | | |
| | | tion of | | | | | | | | | | | |
| | | solubili- | | | | | | | | | | | |
| | | zers† (e.g. | | | | | | | | | | | |
| | | oleic acid | | | | | | | | | | | |
| | | and GDS, | | | | | | | | | | | |
| Comp. | | maisine | Hydrophilic additives* | | | | | | | | | | |
| C- | | 35-1 and | Cremo- | | | | | | Other additives | | | | Ratio of |
| XIV | | capmul, | phor RH | Tween - | | Poloxamer | Co-poly- | Combina- | Anti- | | Flow | Combina- | API: |
| No. | | etc) | 40** | 80*** | SLS | 407**** | mer***** | tions****** | oxidant | Solidifier | agent | tions******* | Solubilizer |
| a | i | 70-80 | — | — | — | — | — | — | 0-1 | 0-10 | 0-5 | 0-15 | ~1:3 |
| b | i | 70-75 | 0-4.5 | — | — | — | — | — | 0-1 | 0-10 | 0-5 | 0-15 | ~1:2.9 |
| | ii | 65-70 | 5-10 | — | — | — | — | — | 0-1 | 0-10 | 0-5 | 0-15 | ~1:2.7 |
| | iii | 60-65 | 10-20 | — | — | — | — | — | 0-1 | 0-10 | 0-5 | 0-15 | ~1:2.5 |
| | iv | 50-60 | 20-30 | — | — | — | — | — | 0-1 | 0-10 | 0-5 | 0-15 | ~1:2.2 |
| | v | 40-50 | 30-40 | — | — | — | — | — | 0-1 | 0-10 | 0-5 | 0-15 | ~1:1.8 |
| | vi | 40-80 | 0-40 | — | — | — | — | — | 0-1 | 0-10 | 0-5 | 0-15 | ~1:1.5-~1:3.2 |
| c | i | 70-75 | — | 0-4.5 | — | — | — | — | 0-1 | 0-10 | 0-5 | 0-15 | ~1:2.9 |
| | ii | 65-70 | — | 5-10 | — | — | — | — | 0-1 | 0-10 | 0-5 | 0-15 | ~1:2.7 |
| | iii | 60-65 | — | 10-20 | — | — | — | — | 0-1 | 0-10 | 0-5 | 0-15 | ~1:2.5 |
| | iv | 50-60 | — | 20-30 | — | — | — | — | 0-1 | 0-10 | 0-5 | 0-15 | ~1:2.2 |
| | v | 40-50 | — | 30-40 | — | — | — | — | 0-1 | 0-10 | 0-5 | 0-15 | ~1:1.8 |
| | vi | 40-80 | — | 0-40 | — | — | — | — | 0-1 | 0-10 | 0-5 | 0-15 | ~1:1.5-~1:3.2 |
| d | i | 70-75 | — | — | 0-4.5 | — | — | — | 0-1 | 0-10 | 0-5 | 0-15 | ~1:2.9 |
| | ii | 65-70 | — | — | 5-10 | — | — | — | 0-1 | 0-10 | 0-5 | 0-15 | ~1:2.7 |
| | iii | 60-65 | — | — | 10-20 | — | — | — | 0-1 | 0-10 | 0-5 | 0-15 | ~1:2.5 |
| | iv | 50-60 | — | — | 20-30 | — | — | — | 0-1 | 0-10 | 0-5 | 0-15 | ~1:2.2 |
| | v | 40-50 | — | — | 30-40 | — | — | — | 0-1 | 0-10 | 0-5 | 0-15 | ~1:1.8 |
| | vi | 40-80 | — | — | 0-40 | — | — | — | 0-1 | 0-10 | 0-5 | 0-15 | ~1:1.5-~1:3.2 |
| e | i | 70-75 | — | — | — | 0-4.5 | — | — | 0-1 | 0-10 | 0-5 | 0-15 | ~1:2.9 |
| | ii | 65-70 | — | — | — | 5-10 | — | — | 0-1 | 0-10 | 0-5 | 0-15 | ~1:2.7 |
| | iii | 60-65 | — | — | — | 10-20 | — | — | 0-1 | 0-10 | 0-5 | 0-15 | ~1:2.5 |
| | iv | 50-60 | — | — | — | 20-30 | — | — | 0-1 | 0-10 | 0-5 | 0-15 | ~1:2.2 |
| | v | 40-50 | — | — | — | 30-40 | — | — | 0-1 | 0-10 | 0-5 | 0-15 | ~1:1.8 |
| | vi | 40-80 | — | — | — | 0-40 | — | — | 0-1 | 0-10 | 0-5 | 0-15 | ~1:1.5-~1:3.2 |
| f | i | 70-75 | — | — | — | — | 0-4.5 | — | 0-1 | 0-10 | 0-5 | 0-15 | ~1:2.9 |
| | ii | 65-70 | — | — | — | — | 5-10 | — | 0-1 | 0-10 | 0-5 | 0-15 | ~1:2.7 |
| | iii | 60-65 | — | — | — | — | 10-20 | — | 0-1 | 0-10 | 0-5 | 0-15 | ~1:2.5 |
| | iv | 50-60 | — | — | — | — | 20-30 | — | 0-1 | 0-10 | 0-5 | 0-15 | ~1:2.2 |
| | v | 40-50 | — | — | — | — | 30-40 | — | 0-1 | 0-10 | 0-5 | 0-15 | ~1:1.8 |
| | vi | 40-80 | — | — | — | — | 0-40 | — | 0-1 | 0-10 | 0-5 | 0-15 | ~1:1.5-~1:3.2 |
| g | i | 70-75 | — | — | — | — | — | 0-4.5 | 0-1 | 0-10 | 0-5 | 0-15 | ~1:2.9 |
| | ii | 65-70 | — | — | — | — | — | 5-10 | 0-1 | 0-10 | 0-5 | 0-15 | ~1:2.7 |
| | iii | 60-65 | — | — | — | — | — | 10-20 | 0-1 | 0-10 | 0-5 | 0-15 | ~1:2.5 |
| | iv | 50-60 | — | — | — | — | — | 20-30 | 0-1 | 0-10 | 0-5 | 0-15 | ~1:2.2 |
| | v | 40-50 | — | — | — | — | — | 30-40 | 0-1 | 0-10 | 0-5 | 0-15 | ~1:1.8 |
| | vi | 40-80 | — | — | — | — | — | 0-40 | 0-1 | 0-10 | 0-5 | 0-15 | ~1:1.5-~1:3.2 |

†Combination of solubilizers can be not limited to oleic acid and GDS or maisine 35-1 and capmul, but 2 or more solubilizers that are listed in Table 2.
*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition D-XIV. Compositions composed of Drug API 30-40%, Combinations of solubilizers, and additives

| | | Compositions (w/w %) | | | | | | | | | | | |
|---|---|---|---|---|---|---|---|---|---|---|---|---|---|
| | | Combina- | | | | | | | | | | | |
| | | tion of | | | | | | | | | | | |
| | | solubili- | | | | | | | | | | | |
| | | zers† (e.g. | | | | | | | | | | | |
| | | oleic acid | | | | | | | | | | | |
| | | and GDS, | | | | | | | | | | | |
| Comp. | | maisine | Hydrophilic additives* | | | | | | | | | | |
| D- | | 35-1 and | Cremo- | | | | | | Other additives | | | | Ratio of |
| XIV | | capmul, | phor RH | Tween - | | Poloxamer | Co-poly- | Combina- | Anti- | | Flow | Combina- | API: |
| No. | | etc) | 40** | 80*** | SLS | 407**** | mer***** | tions****** | oxidant | Solidifier | agent | tions******* | Solubilizer |
| a | i | 60-70 | — | — | — | — | — | — | 0-1 | 0-10 | 0-5 | 0-15 | ~1:1.9 |
| b | i | 65-70 | 0-4.5 | — | — | — | — | — | 0-1 | 0-10 | 0-5 | 0-15 | ~1:1.9 |
| | ii | 60-65 | 5-10 | — | — | — | — | — | 0-1 | 0-10 | 0-5 | 0-15 | ~1:1.8 |
| | iii | 55-60 | 10-15 | — | — | — | — | — | 0-1 | 0-10 | 0-5 | 0-15 | ~1:1.6 |
| | iv | 45-55 | 15-25 | — | — | — | — | — | 0-1 | 0-10 | 0-5 | 0-15 | ~1:1.4 |
| | v | 35-45 | 25-35 | — | — | — | — | — | 0-1 | 0-10 | 0-5 | 0-15 | ~1:1.1 |
| | vi | 35-70 | 0-35 | — | — | — | — | — | 0-1 | 0-10 | 0-5 | 0-15 | ~1:1-~1.2 |
| c | i | 65-70 | — | 0-4.5 | — | — | — | — | 0-1 | 0-10 | 0-5 | 0-15 | ~1:1.9 |
| | ii | 60-65 | — | 5-10 | — | — | — | — | 0-1 | 0-10 | 0-5 | 0-15 | ~1:1.8 |
| | iii | 55-60 | — | 10-15 | — | — | — | — | 0-1 | 0-10 | 0-5 | 0-15 | ~1:1.6 |
| | iv | 45-55 | — | 15-25 | — | — | — | — | 0-1 | 0-10 | 0-5 | 0-15 | ~1:1.4 |
| | v | 35-45 | — | 25-35 | — | — | — | — | 0-1 | 0-10 | 0-5 | 0-15 | ~1:1.1 |
| | vi | 35-70 | — | 0-35 | — | — | — | — | 0-1 | 0-10 | 0-5 | 0-15 | ~1:1-~1.2 |
| d | i | 65-70 | — | — | 0-4.5 | — | — | — | 0-1 | 0-10 | 0-5 | 0-15 | ~1:1.9 |
| | ii | 60-65 | — | — | 5-10 | — | — | — | 0-1 | 0-10 | 0-5 | 0-15 | ~1:1.8 |
| | iii | 55-60 | — | — | 10-15 | — | — | — | 0-1 | 0-10 | 0-5 | 0-15 | ~1:1.6 |
| | iv | 45-55 | — | — | 15-25 | — | — | — | 0-1 | 0-10 | 0-5 | 0-15 | ~1:1.4 |
| | v | 35-45 | — | — | 25-35 | — | — | — | 0-1 | 0-10 | 0-5 | 0-15 | ~1:1.1 |
| | vi | 35-70 | — | — | 0-35 | — | — | — | 0-1 | 0-10 | 0-5 | 0-15 | ~1:1-~1.2 |
| e | i | 65-70 | — | — | — | 0-4.5 | — | — | 0-1 | 0-10 | 0-5 | 0-15 | ~1:1.9 |
| | ii | 60-65 | — | — | — | 5-10 | — | — | 0-1 | 0-10 | 0-5 | 0-15 | ~1:1.8 |
| | iii | 55-60 | — | — | — | 10-15 | — | — | 0-1 | 0-10 | 0-5 | 0-15 | ~1:1.6 |
| | iv | 45-55 | — | — | — | 15-25 | — | — | 0-1 | 0-10 | 0-5 | 0-15 | ~1:1.4 |
| | v | 35-45 | — | — | — | 25-35 | — | — | 0-1 | 0-10 | 0-5 | 0-15 | ~1:1.1 |
| | vi | 35-70 | — | — | — | 0-35 | — | — | 0-1 | 0-10 | 0-5 | 0-15 | ~1:1-~1.2 |
| f | i | 65-70 | — | — | — | — | 0-4.5 | — | 0-1 | 0-10 | 0-5 | 0-15 | ~1:1.9 |
| | ii | 60-65 | — | — | — | — | 5-10 | — | 0-1 | 0-10 | 0-5 | 0-15 | ~1:1.8 |
| | iii | 55-60 | — | — | — | — | 10-15 | — | 0-1 | 0-10 | 0-5 | 0-15 | ~1:1.6 |
| | iv | 45-55 | — | — | — | — | 15-25 | — | 0-1 | 0-10 | 0-5 | 0-15 | ~1:1.4 |
| | v | 35-45 | — | — | — | — | 25-35 | — | 0-1 | 0-10 | 0-5 | 0-15 | ~1:1.1 |
| | vi | 35-70 | — | — | — | — | 0-35 | — | 0-1 | 0-10 | 0-5 | 0-15 | ~1:1-~1.2 |
| g | i | 65-70 | — | — | — | — | — | 0-4.5 | 0-1 | 0-10 | 0-5 | 0-15 | ~1:1.9 |
| | ii | 60-65 | — | — | — | — | — | 5-10 | 0-1 | 0-10 | 0-5 | 0-15 | ~1:1.8 |
| | iii | 55-60 | — | — | — | — | — | 10-15 | 0-1 | 0-10 | 0-5 | 0-15 | ~1:1.6 |
| | iv | 45-55 | — | — | — | — | — | 15-25 | 0-1 | 0-10 | 0-5 | 0-15 | ~1:1.4 |
| | v | 35-45 | — | — | — | — | — | 25-35 | 0-1 | 0-10 | 0-5 | 0-15 | ~1:1.1 |
| | vi | 35-70 | — | — | — | — | — | 0-35 | 0-1 | 0-10 | 0-5 | 0-15 | ~1:1-~1.2 |

†Combination of solubilizers can be not limited to oleic acid and GDS or maisine 35-1 and capmul, but 2 or more solubilizers that are listed in Table 2.
*Hydrophilic additives are not limited to ones listed in this table but include others recited herein.
**Cremophor is not limited to Cremophor RH 40 but Cremophor EL and RH 35.
***Tween is not limited to Tween 80 but Tween 20 and 60.
**** Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

Composition E-XIV. Compositions composed of Drug API 40-50%, Combinations of solubilizers, and additives

| | | Compositions (w/w %) | | | | | | | | | | | |
|---|---|---|---|---|---|---|---|---|---|---|---|---|---|
| | | Combina- | | | | | | | | | | | |
| | | tion of | | | | | | | | | | | |
| | | solubili- | | | | | | | | | | | |
| | | zers† (e.g. | | | | | | | | | | | |
| | | oleic acid | | | | | | | | | | | |
| | | and GDS, | | | | | | | | | | | |
| Comp. | | maisine | Hydrophilic additives* | | | | | | | | | | |
| E- | | 35-1 and | Cremo- | | | | | | Other additives | | | | Ratio of |
| XIV | | capmul, | phor RH | Tween - | | Poloxamer | Co-poly- | Combina- | Anti- | | Flow | Combina- | API: |
| No. | | etc) | 40** | 80*** | SLS | 407**** | mer***** | tions****** | oxidant | Solidifier | agent | tions******* | Solubilizer |
| a | i | 50-60 | — | — | — | — | — | — | 0-1 | 0-10 | 0-5 | 0-15 | ~1:1.2 |
| b | i | 55-60 | 0-4.5 | — | — | — | — | — | 0-1 | 0-10 | 0-5 | 0-15 | ~1:1.3 |
| | ii | 50-55 | 5-10 | — | — | — | — | — | 0-1 | 0-10 | 0-5 | 0-15 | ~1:1.2 |
| | iii | 45-50 | 10-15 | — | — | — | — | — | 0-1 | 0-10 | 0-5 | 0-15 | ~1:1.1 |
| | iv | 40-45 | 15-20 | — | — | — | — | — | 0-1 | 0-10 | 0-5 | 0-15 | ~1:0.9 |
| | v | 30-40 | 20-30 | — | — | — | — | — | 0-1 | 0-10 | 0-5 | 0-15 | ~1:0.8 |
| | vi | 30-60 | 0-30 | — | — | — | — | — | 0-1 | 0-10 | 0-5 | 0-15 | ~1:0.6-~1:1.5 |
| c | i | 55-60 | — | 0-4.5 | — | — | — | — | 0-1 | 0-10 | 0-5 | 0-15 | ~1:1.3 |
| | ii | 50-55 | — | 5-10 | — | — | — | — | 0-1 | 0-10 | 0-5 | 0-15 | ~1:1.2 |
| | iii | 45-50 | — | 10-15 | — | — | — | — | 0-1 | 0-10 | 0-5 | 0-15 | ~1:1.1 |
| | iv | 40-45 | — | 15-20 | — | — | — | — | 0-1 | 0-10 | 0-5 | 0-15 | ~1:0.9 |
| | v | 30-40 | — | 20-30 | — | — | — | — | 0-1 | 0-10 | 0-5 | 0-15 | ~1:0.8 |
| | vi | 30-60 | — | 0-30 | — | — | — | — | 0-1 | 0-10 | 0-5 | 0-15 | ~1:0.6-~1:1.5 |
| d | i | 55-60 | — | — | 0-4.5 | — | — | — | 0-1 | 0-10 | 0-5 | 0-15 | ~1:1.3 |
| | ii | 50-55 | — | — | 5-10 | — | — | — | 0-1 | 0-10 | 0-5 | 0-15 | ~1:1.2 |
| | iii | 45-50 | — | — | 10-15 | — | — | — | 0-1 | 0-10 | 0-5 | 0-15 | ~1:1.1 |
| | iv | 40-45 | — | — | 15-20 | — | — | — | 0-1 | 0-10 | 0-5 | 0-15 | ~1:0.9 |
| | v | 30-40 | — | — | 20-30 | — | — | — | 0-1 | 0-10 | 0-5 | 0-15 | ~1:0.8 |
| | vi | 30-60 | — | — | 0-30 | — | — | — | 0-1 | 0-10 | 0-5 | 0-15 | ~1:0.6-~1:1.5 |
| e | i | 55-60 | — | — | — | 0-4.5 | — | — | 0-1 | 0-10 | 0-5 | 0-15 | ~1:1.3 |
| | ii | 50-55 | — | — | — | 5-10 | — | — | 0-1 | 0-10 | 0-5 | 0-15 | ~1:1.2 |
| | iii | 45-50 | — | — | — | 10-15 | — | — | 0-1 | 0-10 | 0-5 | 0-15 | ~1:1.1 |
| | iv | 40-45 | — | — | — | 15-20 | — | — | 0-1 | 0-10 | 0-5 | 0-15 | ~1:0.9 |
| | v | 30-40 | — | — | — | 20-30 | — | — | 0-1 | 0-10 | 0-5 | 0-15 | ~1:0.8 |
| | vi | 30-60 | — | — | — | 0-30 | — | — | 0-1 | 0-10 | 0-5 | 0-15 | ~1:0.6-~1:1.5 |
| f | i | 55-60 | — | — | — | — | 0-4.5 | — | 0-1 | 0-10 | 0-5 | 0-15 | ~1:1.3 |
| | ii | 50-55 | — | — | — | — | 5-10 | — | 0-1 | 0-10 | 0-5 | 0-15 | ~1:1.2 |
| | iii | 45-50 | — | — | — | — | 10-15 | — | 0-1 | 0-10 | 0-5 | 0-15 | ~1:1.1 |
| | iv | 40-45 | — | — | — | — | 15-20 | — | 0-1 | 0-10 | 0-5 | 0-15 | ~1:0.9 |
| | v | 30-40 | — | — | — | — | 20-30 | — | 0-1 | 0-10 | 0-5 | 0-15 | ~1:0.8 |
| | vi | 30-60 | — | — | — | — | 0-30 | — | 0-1 | 0-10 | 0-5 | 0-15 | ~1:0.6-~1:1.5 |
| g | i | 55-60 | — | — | — | — | — | 0-4.5 | 0-1 | 0-10 | 0-5 | 0-15 | ~1:1.3 |
| | ii | 50-55 | — | — | — | — | — | 5-10 | 0-1 | 0-10 | 0-5 | 0-15 | ~1:1.2 |
| | iii | 45-50 | — | — | — | — | — | 10-15 | 0-1 | 0-10 | 0-5 | 0-15 | ~1:1.1 |
| | iv | 40-45 | — | — | — | — | — | 15-20 | 0-1 | 0-10 | 0-5 | 0-15 | ~1:0.9 |
| | v | 30-40 | — | — | — | — | — | 20-30 | 0-1 | 0-10 | 0-5 | 0-15 | ~1:0.8 |
| | vi | 30-60 | — | — | — | — | — | 0-30 | 0-1 | 0-10 | 0-5 | 0-15 | ~1:0.6-~1:1.5 |

†Combination of solubilizers can be not limited to oleic acid and GDS or maisine 35-1 and capmul, but 2 or more solubilizers that are listed in Table 2.
*Hydrophilic additives are not limited to ones listed in this table but include
**Cremophor is not limited to Cremophor RH 40 but Cremophor EL and RH 35.
***Tween is not limited to Tween 80 but Tween 20 and 60.
****Poloxamer is not limited to Poloxamer 407 but Poloxamer 124, 188, 234, and 335.
*****Co-polymer can be PVP, PMC, or other compounds recited herein.
******Combinations of hydrophilic additives can be 2 or more hydrophilic additives that are listed in this table as well as include others recited herein.
*******Combinations of other additives can be 2 or more other additives that are listed in this table as well as include colorant, coating agent, sweetener, and so on.

The compositions below represent examples of a specific formulation composition for treating virus infected patients.

### Composition F

| | Weight Percent | Quantity Fill | Quantity Fill |
|---|---|---|---|
| | of Fill | Material per | Material per |
| | Pharmaceutical | Hard Gel | Soft Gel |
| | Composition | Capsule | Capsule |
| | (±1%) | (±1%) | (±1%) |
| Ingredient Name | % w/w | mg | mg |
| Testosterone ester | 24 | 183 | 300 |
| or a combination | | | |
| thereof* | | | |
| Oleic Acid, NF | 41 | 308 | 513 |
| Peppermint Oil, NF | 18 | 136 | 225 |
| Polyoxyl 40 | 4 | 30 | 50 |
| Hydrogenated | | | |
| Castor Oil, NF | | | |
| Ascorbyl | 0.2 | 1.5 | 2.5 |
| Palmitate, NF | | | |
| Glyceryl | 12 | 90 | 150 |
| Palmitostearate | | | |
| (Glyceryl | | | |
| Distearate, NF) | | | |
| Total | 100 | 750 | 1241 |

*Drug is not limited to ones in the table, but includes testosterone, dehydroepiandrosterone (DHEA), dehydroepiandrosterone sulfate (DHEA-S), androstenedione (A4), Androsterone, and androstenediol (A5), nandrolone, dimethandrolone, dihydrotestosterone, dihydrotestosterone esters or a combination thereof. It can be untreated (PS < 450 μm), sieved (PS < 450 μm), milled (PS < 150 μm), micronized (1 μm < PS < 25 μm), nanosized (PS < 1 μm), amorphous, or solubilized.

### Composition G

| | Weight Percent | Quantity Fill | Quantity Fill |
|---|---|---|---|
| | of Fill | Material per | Material per |
| | Pharmaceutical | Hard Gel | Soft Gel |
| | Composition | Capsule | Capsule |
| | (±1%) | (±1%) | (±1%) |
| Ingredient Name | % w/w | mg | mg |
| Testosterone ester | 28 | 183 | 350 |
| or a combination | | | |
| thereof* | | | |
| Oleic Acid, NF | 55 | 365 | 688 |
| Polyoxyl 40 | 4 | 26 | 50 |
| Hydrogenated | | | |
| Castor Oil, NF | | | |
| Stearic Acid, NF | 4 | 26 | 50 |
| Glyceryl | 8 | 52 | 100 |
| Palmitostearate | | | |
| (Glyceryl Distearate, | | | |
| NF; Precirol ATO 5) | | | |
| Ascorbyl | | | |
| Palmitate, NF | 0.2 | 1.3 | 2.5 |
| Total | 100 | 654 | 1241 |

*Drug is not limited to ones in the table, but includes testosterone, dehydroepiandrosterone (DHEA), dehydroepiandrosterone sulfate (DHEA-S), androstenedione (A4), Androsterone, and androstenediol (A5), nandrolone, dimethandrolone, dihydrotestosterone, dihydrotestosterone esters or a combination thereof. It can be untreated (PS < 450 μm), sieved (PS < 450 μm), milled (PS < 150 μm), micronized (1 μm < PS < 25 μm), nanosized (PS < 1 μm), amorphous, or solubilized.

### Composition H, I, K

| | Composition (weight %) | | |
|---|---|---|---|
| Composition No. | H | I | K |
| Components | | | |
| Testosterone ester or | 10-30 | 10-30 | 10-30 |
| a combination thereof* | | | |
| Lipophilic additive [e.g. | 55-80 | 55-80 | 55-80 |
| surfactant of HLB < 10 | | | |
| such as mono- or di- or | | | |
| tri- glyceride of fatty | | | |
| acid or fatty acid] | | | |
| Hydrophilic additive | 0-20 | 0-20 | 0-20 |
| (e.g. Surfactant with | | | |
| HLB > 10 such as | | | |
| Cremophor RH40) | | | |
| Adjuvant | q.s. | q.s | q.s. |

*Drug is not limited to ones in the table, but includes testosterone, dehydroepiandrosterone (DHEA), dehydroepiandrosterone sulfate (DHEA-S), androstenedione (A4), Androsterone, and androstenediol (A5), nandrolone, dimethandrolone, dihydrotestosterone, dihydrotestosterone esters or a combination thereof. It can be untreated (PS < 450 μm), sieved (PS < 450 μm), milled (PS < 150 μm), micronized (1 μm < PS < 25 μm), nanosized (PS < 1 μm), amorphous, or solubilized.

### Testosterone Undecanoate (TU) Example Compositions

### TU Example 1

A testosterone undecanoate containing composition was prepared by using the components set forth in Table I. The composition is prepared by weighing all of the components, except the testosterone undecanoate, into a clean stainless steel container and mixed together at about 50° C. to about 70° C., using a stirrer. The testosterone undecanoate (TU) is added and stirred into the mixture of other components until the testosterone undecanoate dissolves. A predetermined quantity of this fill material is disposed into a capsule (for example, hard gelatin capsule) to get the required testosterone undecanoate dose per dosage unit. The capsules are allowed to cool at room temperature, banded (if required) and packaged in a HDPE bottle and tightly closed with an appropriate lid.

TABLE I

| | Composition |
|---|---|
| TU Example 1 | mg/capsule |
| Testosterone Undecanoate | 200 |
| Solubilizer (e.g. Glycerides | 725 |
| of coconut oil; Capmul ® MCM) | |
| Dispersant (e.g. lauroglycol) | 300 |
| Dispersant (polyoxyl 40 hydrogenated | 50 |
| castor oil or Cremophor ® RH40) | |
| TU-loading (wt %) of capsule fill = 15.7% | |

### TU Example 2

A testosterone undecanoate containing composition was prepared similarly to the method described in Example 1 using the components set forth in Table II.

TABLE II

| | Composition |
|---|---|
| TU Example 2 | mg/capsule |
| Testosterone Undecanoate | 225 |
| Solubilizer (e.g. Maize oil glyceride) | 260 |
| Dispersant (e.g. lauroglycol) | 665 |
| TU-loading (wt %) of capsule fill ~19.6% | |

### TU Examples 3 & 4—Testosterone Undecanoate Composition

Testosterone undecanoate containing composition were prepared similarly to the method described in Example 1 using the components set forth in Tables III and IV

TABLE III

| | Composition |
|---|---|
| TU Example 3 | mg/capsule |
| Testosterone Undecanoate | 200 |
| Solubilizer (e.g. Glycerides | 600 |
| of coconut oil; Capmul ® MCM) | |
| TU-loading (wt %) of capsule fill = 25% | |

TABLE IV

| | Composition |
|---|---|
| TU Example 4 | mg/capsule |
| Testosterone Undecanoate | 180 |
| Solubilizer (Maize oil glyceride, | 600 |
| Maisine 35-1) | |
| TU-loading (wt %) of capsule fill = 23% | |

### TU Example 5

A testosterone undecanoate containing composition was prepared similarly to the method described in Example 1 using the components set forth in Table V

TABLE V

| | Composition |
|---|---|
| TU Composition 5 | mg/capsule |
| Testosterone Undecanoate | 240 |
| Solubilizer (e.g. Glycerides of coconut oil; | 200 |
| Capmul ® MCM) | |
| Solubilizer (e.g. α-tocopherol) | 490 |
| Dispersant (for e.g. polyoxyl castor oil or | 100 |
| Cremophor ® EL) | |
| TU-Loading per caps TU-loading (wt %) | |
| of capsule fill = 23.3% | |

### TU Example 6

A testosterone undecanoate containing composition was prepared by using the components set forth in Table VI and a method similar to that described in Example 1.

TABLE VI

| TU Example 6 | Composition mg/capsule |
|---|---|
| Testosterone Undecanoate | 200 |
| Solubilizer (e.g. Maize oil | 490 |
| glycerides) | |
| Dispersant (e.g. polysorbate 80) | 25 |
| Solidifying agent (e.g. polyethylene | 45 |
| glycol, 8000 or PEG 8000) | |
| TU-Loading per capsule = 26.3% | |

### TU Example 7

A testosterone undecanoate containing composition was prepared by using the components set forth in Table VII and a method similar to that described in Example 1.

TABLE VII

| | Composition |
|---|---|
| TU Example 7 | mg/capsule |
| Testosterone Undecanoate | 240 |
| Solubilizer (e.g.Maize oil glycerides) | 325 |
| Solubilizer (e.g. oleic acid) | 125 |
| Solubilizer (e.g. Benzyl Alcohol) | 50 |
| Solubilizer (e.g. α-tocopherol) | 75 |
| Solidifying agent (e.g. PEG 8000) | 45 |
| TU-Loading per capsule = 28% | |

### TU Example 8

A testosterone undecanoate containing composition was prepared by using the components set forth in Table VIII and a method similar to that described in Example 1.

TABLE VIII

| | Composition |
|---|---|
| TU Example 8 | mg/capsule |
| Testosterone Undecanoate | 240 |
| Solubilizer (e.g. oleic acid) | 400 |
| Solidifying agent-(e.g. PEG 8000) | 45 |
| TU-loading (wt %) of capsule fill = 35% | |

### TU Example 9

A testosterone undecanoate containing composition was prepared using the components set forth in Table IX and a method similar to that described in Example 1.

TABLE IX

| | Composition |
|---|---|
| TU Example 9 | mg/capsule |
| Testosterone Undecanoate | 240 |
| Solubilizer (e.g. Maize oil glycerides) | 400 |
| Solubilizer (e.g. α-tocopherol) | 24 |
| Solidifying agent-(e.g. Glyceryl distearate; | 25 |
| Percirol ® ATO 5) | |
| TU-loading (wt %) of capsule fill = 34.8% | |

### TU Example 10

A testosterone undecanoate containing composition can be prepared by using the components set forth in Table X by a method as follows: The required quantity of the glyceryl distearate or glyceryl monostearate and the PEG 8000 are placed in a stainless steel container and heated to about 50 to 70° C. to get a molten mixture. The testosterone undecanoate is added and stirred till it completely dissolves. A predetermined weight of the molten mixture is disposed into capsules and allowed to congeal at room temperature, banded and packed.

TABLE X

| | Composition |
|---|---|
| TU Example 10 | mg/capsule |
| Testosterone Undecanoate | 100 |
| Glyceryl distearate (Percirol ® ATO 5) | 200 |
| or glyceryl monostearate | |
| PEG 8000 | 50 |

The oral dosage capsules of Example 10, which contains non-dissolved TU, can provide, upon single administration along with food to a human male, a testosterone undecanoate AUC that is about 20% higher as compared to a composition that does not include the glyceryl distearate (Percirol® ATO 5) or glyceryl monostearate.

The composition of Example 10 can also be optionally modified so that a dispersant such as a disintegrating agent (e.g. Crospovidone at about 150 mg for every 100 mg TU dose) can be uniformly suspended under stirring in the molten testosterone undecanoate solution. This suspension can be further allowed to cooled and passed through ASTM 30 mesh get granulates or particulates which can be either filled in a capsule or compressed to a tablet.

### TU Example 11

A testosterone undecanoate containing composition wherein at least 50% of the testosterone undecanoate is dissolved is prepared by using the components set forth in Table XI and a method similar to that described under Example 1.

TABLE XI

| | Composition |
|---|---|
| TU Example 11 | mg/capsule |
| Testosterone Undecanoate | 225 |
| Solubilizer (e.g. Castor Oil) | 350 |
| Dispersant (e.g. lauroglycol); | 180 |
| Solidifying agent (e.g. PEG 8000) | 45 |
| TU-loading (wt %) of capsule fill = 28.1% | |

### TU Examples 12-19

Testosterone undecanoate formulations of Examples 12 through 19 were prepared by using the components set forth in Table XII and by a method similar to that described under Example 1. Additionally, indicated amounts of the respective formulations were filled into hard gelatin capsules and the testosterone undecanoate release from capsules is measured using a USP Type-II apparatus at about 100 rpm in about 1000 mL of 8% w/w solution of Triton X100 in water, maintained at about 37° C. The results of the release testing are also shown in Table XII.

TABLE XII

| Capsule | Composition mg/capsule | | | | | | | | |
|---|---|---|---|---|---|---|---|---|---|
| Components/ | Example | Example | Example | Example | Example | Example | Example | Example | Example- |
| Attributes | 12 | 13 | 14 | 15 | 15A | 16 | 17 | 18 | 19 |
| Testosterone | 40 | 40 | 75 | 75 | 90 | 75 | 75 | 75 | 125 |
| Undecanoate | | | | | | | | | |
| Oleic Acid | 227 | — | — | — | | — | — | — | |
| Castor oil | | 175 | — | — | | 455 | — | — | |
| Lauroglycol | | 115 | — | — | | — | — | — | |
| Labrafil | | | | | | 80 | — | — | |
| M2125CS | | | | | | | | | |
| Maize oil | | — | 455 | 455 | 315 | — | 316 | 316 | 515 |
| glycerides | | | | | | | | | |
| (Maisine 35-1) | | | | | | | | | |
| Polyoxyl 40 | | — | 130 | 80 | 35 | — | 79 | 54 | 112 |
| Hydrogenated | | | | | | | | | |
| Castor Oil, | | | | | | | | | |
| (Cremophor | | | | | | | | | |
| RH40) | | | | | | | | | |
| Glyceryl | | — | — | 50 | 60 | — | — | 25 | |
| distearate | | | | | | | | | |
| (Percirol ATO 5) | | | | | | | | | |
| Polyethylene | | — | — | — | | 48 | 30 | 30 | 48 |
| Glycol 8000, | | | | | | | | | |
| Total mg per | 267 | 330 | 660 | 660 | 500 | 660 | 500 | 500 | 800 |
| capsule (mg) | | | | | | | | | |
| TU-loading | 15% | 12% | 11.3% | 11.3% | 18% | 11.3% | 15% | 15% | 15.6% |
| (wt %) of capsule | | | | | | | | | |
| fill | | | | | | | | | |
| TU released in | ~100% | ~100% | ~100% | 30% | 30% | ~78% | 85% | 32% | 80% |
| 30 minutes (%) | | | | | | | | | |
| Time for 75% | <120 | <120 | <120 | <120 | <120 | <120 | <120 | <120 | <120 |
| TU release | | | | | | | | | |
| (minutes) | | | | | | | | | |

### TU Examples 20-25

Testosterone undecanoate formulations of Examples 20 through 25 were prepared by using the components set forth in Table XIII and by a method similar to that described under Example 1. Additionally, indicated amounts of the respective formulations were filled into hard gelatin capsules and the testosterone undecanoate release from capsules was tested in about 1000 mL of 8% w/w solution of Triton X100 in water, maintained at about 37° C., using a USP Type-II apparatus at about 100 rpm. The results of the release testing are also shown in Table XIII.

TABLE XIII

| Capsule Components/ | Composition mg/capsule | | | | | |
|---|---|---|---|---|---|---|
| Attributes | Example 20 | Example 21 | Example 22 | Example 23 | Example 24 | Example 25 |
| Testosterone Undecanoate | 200 | 200 | 240 | 240 | 240 | 240 |
| (TU) | | | | | | |
| Maize oil glycerides | — | 490 | 464 | 464 | | 304 |
| (Maisine 35-1) | | | | | | |
| Coconut oil glycerides | — | — | — | — | 400 | — |
| (Capmul MCM) | | | | | | |
| Alpha-tocopherol | 510 | — | — | 50 | — | 50 |
| Benzyl alcohol | — | | — | — | 25 | 50 |
| Polyoxyl 40 Hydrogenated | — | 25 | — | — | — | — |
| Castor Oil, | | | | | | |
| Polyoxyl 35 Castor Oil, | 45 | — | — | — | — | — |
| Polyethylene Glycol 8000, | 45 | 45 | — | 46 | 65 | 41 |
| USP | | | | | | |
| Total Fill wt. per capsule | 800 | 760 | 704 | 800 | 730 | 685 |
| (mg) | | | | | | |
| TU-loading (wt %) of | 25 | 26.3 | 34.0 | 30.0 | 32.9 | 35.0 |
| capsule fill | | | | | | |
| TU released in 30 minutes | 43% | 62% | 38% | 32% | <75% | <75% |
| Time for 75% TU release | <120 | <120 | <120 | <120 | <120 | <120 |
| (minutes) | | | | | | |

### TU Examples 26-29

Testosterone undecanoate formulations of Examples 26 through 29 were prepared by using the components set forth in Table XIV and a method similar to that described under Example 1. Additionally, indicated amounts of the respective formulations were filled into hard gelatin capsules and the testosterone undecanoate released from capsules was tested in about 1000 mL of 8% w/w solution of Triton X100 in water, maintained at about 37° C., using a USP Type-II apparatus at about 100 rpm. The results of the release testing are also shown in Table XIV.

TABLE XIV

| | Composition mg/capsule | | | |
|---|---|---|---|---|
| | Example | Example | Example | Example |
| Capsule Components/Attributes | 26 | 27 | 28 | 29 |
| Testosterone Undecanoate | 250 | 250 | 250 | 250 |
| Maize oil glycerides (Maisine | 486 | 937 | 410 | 939 |
| 35-1) | | | | |
| Polyoxyl 40 Hydrogenated | 25 | 213 | 69 | 144 |
| Castor Oil (Cremophor RH40) | | | | |
| Glyceryl distearate (Percirol | — | — | 32 | 67 |
| ATO 5) | | | | |
| Polyethylene Glycol 8000, | 39 | — | 39 | — |
| Total mg per capsule | 800 | 1400 | 800 | 1400 |
| TU-loading (wt %) of capsule | 31.3 | 18 | 31.3 | 18 |
| fill | | | | |
| TU released in 30 minutes | <75% | <75% | <75% | <75% |
| Time for 75% TU release | <120 | <120 | <120 | <120 |
| (minutes) | | | | |

Note:
Examples 27 and 29 can optionally be disposed in a delayed release capsule

### TU Examples 30-35

Testosterone undecanoate formulations Examples 30 through 35 can be prepared by using the components set forth in Table XV and by a method similar to that described in Example 1. Additionally, indicated amounts of the respective formulations can be encapsulated in gelatin capsules and the testosterone undecanoate release from the capsules tested in about 1000 mL of 8% w/w solution of Triton X100 in water, maintained at about 37° C., using a USP Type-II apparatus at about 100 rpm. The results of the release testing are also shown in Table XV.

TABLE XV

| Capsule Components/ | Composition mg/capsule | | | | | |
|---|---|---|---|---|---|---|
| Attributes | Example 30 | Example 31 | Example 32 | Example 33 | Example 34 | Example 35 |
| Testosterone | 368 | 320 | 490 | 240 | 40 | 300 |
| Undecanoate | | | | | | |
| Maize oil glycerides | 900 | 370 | 620 | 404 | — | — |
| (Maisine 35-1) | | | | | | |
| Castor Oil | — | — | — | — | 175 | 276 |
| Lauroglycol | — | — | | — | 115 | 184 |
| Tocopherol | — | — | 102 | — | — | |
| Benzyl alcohol | — | — | 102 | — | — | |
| Polyoxyl 40 | 46 | 25 | — | — | — | — |
| Hydrogenated Castor Oil, | | | | | | |
| Polyethylene Glycol | 86 | 45 | 86 | 41 | — | — |
| 8000, USP | | | | | | |
| TU-loading (wt %) of | 26.3 | 36.3 | 35.0 | 35.0 | 12.0 | 39.5 |
| capsule fill | | | | | | |
| Total Fill wt. per capsule | 1400 | 760 | 1400 | 685 | 330 | 760 |
| TU released in 30 | 62% | 62% | <75% | <75% | ~100% | <75% |
| minutes | | | | | | |
| Time for 75% TU release | <120 | >120 | <120 | <120 | <120 | >120 |
| (minutes) | | | | | | |

Examples 30 through 35 demonstrate the importance of the choice of the solubilizers of the current invention and their levels to achieve greater testosterone undecanoate loading and yet maintain the solubilization of the testosterone undecanoate in the composition and/or the dosage form.

### TU Examples 36 and 37

The compositions of the current invention can be further adsorbed onto one or more substrate materials such as, for example, lactose, magnesium aluminosilicate, colloidal silicon dioxide, starch, microcrystalline cellulose, alkyl celluloses etc., whereby a free flowing powder/granule form is obtained which can be used as a granules, or disposed into capsule, or pressed into tablet. The amount of the substrate material can be from about 15% to about 40% of the weight of the composition. In one embodiment, the amount of the substrate material can be from about 20% to about 35% of the weight of the formed granule or powder. The method of making such adsorbed testosterone undecanoate compositions can include pouring the liquid compositions on the substrate material under and continuous mixing at room temperature or at about 50° C.-70° C., depending on the composition. After cooling, the adsorbed composition can be disposed into capsule or pressed into tablet. Table XVI illustrates examples of the freely flowable adsorbed solubilized testosterone undecanoate compositions.

TABLE XVI

| | Composition (% w/w) | |
|---|---|---|
| Components/Attributes | Example 36 | Example 37 |
| Testosterone Undecanoate | 16 | 16 |
| Maize oil glycerides (Maisine 35-1) | 50 | — |
| Castor Oil | — | 45 |
| Sorbitan monolaurate (Span ® 20) | — | 5 |
| Tocopherol | 1 | — |
| Glycerylpalmito stearate | 5 | — |
| Polyoxyl 40 Hydrogenated Castor Oil, | 3 | — |
| Polyethylene Glycol 8000, USP | — | 4 |
| Magnesium aluminosilicate (Neusilin ® US2) | 25 | 30 |

### TU Examples 39, 39A, and 40 to 47

Testosterone undecanoate-containing compositions (capsule fill material) were prepared by using the components set forth in Tables XVIII and XIX. It should be noted that Example 40 and 47 are expressed as wt % of Examples 17 and 12 respectively. The compositions are prepared by weighing all of the components, except the testosterone undecanoate, into a clean stainless steel container and mixed together at about 50° C. to about 70° C., using a stirrer. The testosterone undecanoate (TU) is added and stirred into the mixture of other components until the testosterone undecanoate dissolves. A predetermined quantity of the “capsule fill” was disposed into a capsule (for example, hard gelatin capsule) to get the required testosterone undecanoate dose per dosage unit. The capsules are allowed to cool at room temperature, banded (if required) and packaged in a HDPE bottle and tightly closed with an appropriate lid.

TABLE-XVIII

| | Example | Example | Example | Example | Example |
|---|---|---|---|---|---|
| Capsule Components/Attributes | 39 | 39A | 40 | 41 | 42 |
| Testosterone Undecanoate, wt % | 14 | 14 | 15 | 18 | 18 |
| Monoglycerides (e.g. maize oil monoglycerides, Maisine 35-1), wt % | 64 | 75 | 63 | 68 | 68 |
| Hydrophilic surfactants (e.g. Cremophor RH40, Cremophor EL), wt % | 16 | 5 | 16 | 8 | 14 |
| Fatty acids (e.g. linoleic acid, Linolenic acid, Oleic Acid), wt % | — | — | — | — | — |
| Triglyceride (e.g. castor oil, maize oil, borage seed oil, lauroglycol, | — | — | — | — | — |
| corn oil etc.), wt % | | | | | |
| Lipophilic surfactant (e.g. propylene glycol monolaurate), wt % | — | — | — | — | — |
| Solidifying agent (e.g. polyethylene glycol 8000), wt % | 6 | 6 | 6 | 6 | — |
| % TU dissolved at RT | 70-75 | 85-92 | 65-70 | 50-55 | 55-60 |
| % TU undissolved fraction at RT | 25-30 | 8-15 | 30-35 | 45-50 | 40-45 |

*It is noted that the compositions can also include one or more components such as anti-oxidants (e.g. BHA, BHT, Ascorbyl palmitate, vitamin E or combinations), flavorings etc.

TABLE-XIX

| | Example | Example | Example | Example | Example |
|---|---|---|---|---|---|
| Capsule Components/Attributes | 43 | 44 | 45 | 46 | 47 |
| Testosterone Undecanoate, wt % | 25 | 18 | 20 | 12 | 12 |
| Monoglycerides (e.g. maize oil monoglycerides, Maisine 35-1), wt % | 70 | — | — | — | — |
| Hydrophilic surfactants (e.g. Cremophor RH40, Cremophor EL), wt % | 5 | 17 | 16 | | — |
| Fatty acids (e.g. linoleic acid, Linolenic acid, Oleic Acid), wt % | — | 53 | 52 | 88 | — |
| Triglyceride (e.g. castor oil, maize oil, borage seed oil, lauroglycol, | — | 12 | 12 | — | 53 |
| corn oil etc.), wt % | | | | | |
| Lipophilic surfactant (e.g. propylene glycol monolaurate), wt % | — | — | — | — | 35 |
| Solidifying agent (e.g. polyethylene glycol 8000), wt % | — | — | — | — | — |
| % TU dissolved at RT | 40-45 | 100 | 100 | 100 | 100 |
| % TU undissolved fraction at RT | 55-60 | 0 | 0 | 0 | 0 |

*It is noted that the compositions can also include one or more components such as anti-oxidants (e.g. BHA, BHT, Ascorbyl palmitate, vitamin E or combinations), flavorings etc.

The compositions of tables XVIII and XIX include descriptions of the amounts of testosterone undecanoate that is dissolved at room temperature (RT). The percent (%) of the undissolved testosterone undecanoate in the composition at RT can range from about 5 wt % to about 60 wt %. It is noteworthy those Examples 44 to 47 do not include a solidifying agent and that about 100 wt % of the testosterone undecanoate is dissolved in these composition. All Examples, except Examples 44 to 47 are free of ionizable fatty acids such as oleic acid.

### Testosterone Dodecanoate (TD) Example Compositions

### TD Example 1

A testosterone dodecanoate containing composition was prepared by using the components set forth in Table I-TD. The composition is prepared by weighing all of the components, except the testosterone dodecanoate, into a clean stainless steel container and mixed together at about 50° C. to about 70° C., using a stirrer. The testosterone dodecanoate (TD) is added and stirred into the mixture of other components until the testosterone dodecanoate dissolves. A predetermined quantity of this fill material is disposed into a capsule (for example, hard gelatin capsule) to get the required testosterone dodecanoate dose per dosage unit. The capsules are allowed to cool at room temperature, banded (if required) and packaged in a HDPE bottle and tightly closed with an appropriate lid.

TABLE I-TD

| | Composition |
|---|---|
| TD Example 1 | mg/capsule |
| Testosterone Dodecanoate | 100-200 |
| Solubilizer (e.g. Glycerides of coconut | 500-750 |
| oil; Capmul ® MCM) | |
| Dispersant (e.g. lauroglycol) | 200-300 |
| Dispersant (polyoxyl 40 hydrogenated | 30-50 |
| castor oil or Cremophor ® RH40) | |
| TD-loading (wt %) of capsule fill = 15%-20% | |

### TD Example 2

A testosterone dodecanoate containing composition was prepared similarly to the method described in TD Example 1 using the components set forth in Table II-TD.

TABLE II-TD

| | Composition |
|---|---|
| TD Example 2 | mg/capsule |
| Testosterone Dodecanoate | 200-300 |
| Solubilizer (e.g. Maize oil glyceride) | 200-600 |
| Dispersant (e.g. lauroglycol) | 100-300 |
| TD-loading (wt %) of capsule fill = 20%-25% | |

### TD Examples 3 & 4

Testosterone dodecanoate containing composition were prepared similarly to the method described in TD Example 1 using the components set forth in Tables III-TD and IV-TD

TABLE III-TD

| | Composition |
|---|---|
| TD Example 3 | mg/capsule |
| Testosterone Dodecanoate | 200-400 |
| Solubilizer (e.g. Glycerides of coconut oil; | 200-800 |
| Capmul ® MCM) | |
| TD-loading (wt %) of capsule fill = 25%-30% | |

TABLE IV-TD

| | Composition |
|---|---|
| TD Example 4 | mg/capsule |
| Testosterone Dodecanoate | 200-225 |
| Solubilizer (Maize oil | 500-600 |
| glyceride, Maisine 35-1) | |

TD-loading (wt %) of capsule fill = 23%

### TD Example 5

A testosterone dodecanoate containing composition was prepared similarly to the method described in TD Example 1 using the components set forth in Table V-TD

TABLE V-TD

| | Composition |
|---|---|
| TD Composition 5 | mg/capsule |
| Testosterone Dodecanoate | 240-300 |
| Solubilizer (e.g. Glycerides of | 150-300 |
| coconut oil; Capmul ® MCM) | |
| Solubilizer (e.g. α-tocopherol) | 300-500 |
| Dispersant (for e.g. polyoxyl | 50-100 |
| castor oil or Cremophor ® EL) | |

TD-Loading per caps TU-loading (wt %) of capsule fill = 23.3%

### TD Example 6

A testosterone dodecanoate containing composition was prepared by using the components set forth in Table VI-TD and a method similar to that described in TD Example 1.

TABLE VI-TD

| | Composition |
|---|---|
| TD Example 6 | mg/capsule |
| Testosterone Dodecanoate | 200-250 |
| Solubilizer (e.g. Maize oil | 400-500 |
| glycerides) | |
| Dispersant (e.g. polysorbate 80) | 20-25 |
| Solidifying agent (e.g. polyethylene | 35-50 |
| glycol, 8000 or PEG 8000) | |

TD-Loading per capsule = 26.3%

### TD Example 7

A testosterone dodecanoate containing composition was prepared by using the components set forth in Table VII-TD and a method similar to that described in TD Example 1.

TABLE VII-TD

| | Composition |
|---|---|
| TD Example 7 | mg/capsule |
| Testosterone Dodecanoate | 240-300 |
| Solubilizer (e.g. Maize oil glycerides) | 250-350 |
| Solubilizer (e.g. oleic acid) | 100-125 |
| Solubilizer (e.g. Benzyl Alcohol) | 40-50 |
| Solubilizer (e.g. α-tocopherol) | 60-75 |
| Solidifying agent (e.g. PEG 8000) | 35-45 |

TD-Loading per capsule = 28%

### TD Example 8

A testosterone dodecanoate containing composition was prepared by using the components set forth in Table VIII-TD and a method similar to that described in TD Example 1.

TABLE VIII-TD

| | Composition |
|---|---|
| TD Example 8 | mg/capsule |
| Testosterone dodecanoate | 240-300 |
| Solubilizer (e.g. oleic acid) | 300-400 |
| Solidifying agent-(e.g. PEG 8000) | 35-45 |

TD-loading (wt %) of capsule fill = 35%

### TD Example 9

A testosterone dodecanoate containing composition was prepared using the components set forth in Table IX-TD and a method similar to that described in TD Example 1.

TABLE IX-TD

| | Composition |
|---|---|
| TD Example 9 | mg/capsule |
| Testosterone dodecanoate | 240-300 |
| Solubilizer (e.g. Maize oil glycerides) | 300-400 |
| Solubilizer (e.g. α-tocopherol) | 20-24 |
| Solidifying agent-(e.g. Glyceryl | 20-25 |
| distearate;Percirol ® ATO 5) | |

TD-loading (wt %) of capsule fill = 34.8%

### TD Example 10

A testosterone dodecanoate containing composition can be prepared by using the components set forth in Table X-TD by a method as follows: The required quantity of the glyceryl distearate or glyceryl monostearate and the PEG 8000 are placed in a stainless steel container and heated to about 50 to 70° C. to get a molten mixture. The testosterone dodecanoate is added and stirred till it completely dissolves. A predetermined weight of the molten mixture is disposed into capsules and allowed to congeal at room temperature, banded and packed.

TABLE X-TD

| | Composition |
|---|---|
| TD Example 10 | mg/capsule |
| Testosterone Dodecanoate | 100-150 |
| Glyceryl distearate | 150-200 |
| (Percirol ® ATO 5) | |
| or glyceryl monostearate | |
| PEG 8000 | 50-100 |

The oral dosage capsules of TD Example 10, which contains non-dissolved TD, can provide, upon single administration along with food to a human male, a testosterone dodecanoate AUC that is about 20% higher as compared to a composition that does not include the glyceryl distearate (Percirol® ATO 5) or glyceryl monostearate. The composition of TD Example 10 can also be optionally modified so that a dispersant such as a disintegrating agent (e.g. Crospovidone at about 150 mg for every 125 mg TD dose) can be uniformly suspended under stirring in the molten testosterone dodecanoate solution. This suspension can be further allowed to cooled and passed through ASTM 30 mesh get granulates or particulates which can be either filled in a capsule or compressed to a tablet.

### TD Example 11

A testosterone dodecanoate containing composition wherein at least 50% of the testosterone dodecanoate is dissolved is prepared by using the components set forth in Table XI-TD and a method similar to that described under TD Example 1.

TABLE XI-TD

| | Composition |
|---|---|
| TD Example 11 | mg/capsule |
| Testosterone Dodecanoate | 225-300 |
| Solubilizer (e.g. Castor Oil) | 250-350 |
| Dispersant (e.g. lauroglycol); | 150-180 |
| Solidifying agent (e.g. PEG 8000) | 35-45 |

TU-loading (wt %) of capsule fill = 28.1%

### TD Examples 12-19

Testosterone dodecanoate formulations of TD Examples 12 through 19 were prepared by using the components set forth in Table XII and by a method similar to that described under TD Example 1. Additionally, indicated amounts of the respective formulations were filled into hard gelatin capsules and the testosterone dodecanoate release from capsules is measured using a USP Type-II apparatus at about 100 rpm in about 1000 mL of 8% w/w solution of Triton X100 in water, maintained at about 37° C. The results of the release testing are also shown in Table XII-TD.

TABLE XII-TD

| | Composition mg/capsule | | | | | | | | |
|---|---|---|---|---|---|---|---|---|---|
| Capsule | TD | TD | TD | TD | TD | TD | TD | TD | TD |
| Components/ | Example | Example | Example | Example | Example | Example | Example | Example | Example- |
| Attributes | 12 | 13 | 14 | 15 | 15A | 16 | 17 | 18 | 19 |
| Testosterone | 40-50 | 40-50 | 75-100 | 75-100 | 90-125 | 75-100 | 75-100 | 75-100 | 125-200 |
| dodecanoate | | | | | | | | | |
| Oleic Acid | 200-227 | — | — | — | | — | — | — | |
| Castor oil | | 150-200 | — | — | | 400-455 | — | — | |
| Lauroglycol | | 100-115 | — | — | | — | — | — | |
| Labrafil | | | | | | 60-80 | — | — | |
| M2125CS | | | | | | | | | |
| Maize oil glycerides | | — | 400-455 | 400-455 | 280-315 | — | 280-316 | 280-316 | 400-515 |
| (Maisine 35-1) | | | | | | | | | |
| Polyoxyl 40 | | — | 100-130 | 60-80 | 30-35 | — | 65-79 | 45-54 | 100-112 |
| Hydrogenated Castor Oil, | | | | | | | | | |
| (Cremophor RH40) | | | | | | | | | |
| Glyceryl distearate | | — | — | 40-50 | 50-60 | — | — | 20-25 | |
| (Percirol ATO 5) | | | | | | | | | |
| Polyethylene | | — | — | — | | 40-48 | 25-30 | 25-30 | 40-48 |
| Glycol 8000, | | | | | | | | | |
| Total mg per | 240-267 | 250-400 | 600-750 | 600-750 | 450-600 | 600-750 | 450-600 | 450-600 | 700-850 |
| capsule (mg) | | | | | | | | | |
| TU-loading (wt %) of | 15-18% | 12-15% | 11-15% | 11-15% | 18-25% | 11-15% | 15-20% | 15-20% | 15-20% |
| capsule fill | | | | | | | | | |
| TU released in | ~100% | ~100% | ~100% | 30% | 30% | ~78% | 85% | 32% | 80% |
| 30 minutes (%) | | | | | | | | | |
| Time for 75% TU release | <120 | <120 | <120 | <120 | <120 | <120 | <120 | <120 | <120 |
| (minutes) | | | | | | | | | |

### TD Examples 20-25

Testosterone dodecanoate formulations of TD Examples 20 through 25 were prepared by using the components set forth in Table XIII-TD and by a method similar to that described under TD Example 1. Additionally, indicated amounts of the respective formulations were filled into hard gelatin capsules and the testosterone dodecanoate release from capsules was tested in about 1000 mL of 8% w/w solution of Triton X100 in water, maintained at about 37° C., using a USP Type-II apparatus at about 100 rpm. The results of the release testing are also shown in Table XIII-TD.

TABLE XIII-TD

| | Composition mg/capsule | | | | | |
|---|---|---|---|---|---|---|
| Capsule Components/ | TD Example | TD Example | TD Example | TD Example | TD Example | TD Example |
| Attributes | 20 | 21 | 22 | 23 | 24 | 25 |
| Testosterone Dodecanoate | 200-250 | 200-250 | 240-300 | 240-300 | 240-300 | 240-300 |
| (TD) | | | | | | |
| Maize oil glycerides | — | 400-490 | 400-464 | 400-464 | | 250-304 |
| (Maisine 35-1) | | | | | | |
| Coconut oil glycerides | — | — | — | — | 300-400 | — |
| (Capmul MCM) | | | | | | |
| Alpha-tocopherol | 400-510 | — | — | 40-50 | — | 40-50 |
| Benzyl alcohol | — | | — | — | 20-25 | 40-50 |
| Polyoxyl 40 Hydrogenated | — | 20-25 | — | — | — | — |
| Castor Oil, | | | | | | |
| Polyoxyl 35 Castor Oil, | 40-45 | — | — | — | — | — |
| Polyethylene Glycol 8000, | 40-45 | 40-45 | — | 46 | 65 | 41 |
| USP | | | | | | |
| Total Fill wt. per capsule | 700-850 | 700-850 | 650-750 | 700-850 | 650-800 | 650-800 |
| (mg) | | | | | | |
| TD-loading (wt %) of | 25-30 | 25-30 | 30-40 | 30-35 | 30-35 | 35-40 |
| capsule fill | | | | | | |
| TU released in 30 minutes | 43% | 62% | 38% | 32% | <75% | <75% |
| Time for 75% TU release | <120 | <120 | <120 | <120 | <120 | <120 |
| (minutes) | | | | | | |

### TD Examples 26-29

Testosterone dodecanoate formulations of TD Examples 26 through 29 were prepared by using the components set forth in Table XIV-TD and a method similar to that described under TD Example 1. Additionally, indicated amounts of the respective formulations were filled into hard gelatin capsules and the testosterone dodecanoate released from capsules was tested in about 1000 mL of 8% w/w solution of Triton X100 in water, maintained at about 37° C., using a USP Type-II apparatus at about 100 rpm. The results of the release testing are also shown in Table XIV-TD.

TABLE XIV-TD

| | Composition mg/capsule | | | |
|---|---|---|---|---|
| | TD | TD | TD | TD |
| Capsule Components/ | Example | Example | Example | Example |
| Attributes | 26 | 27 | 28 | 29 |
| Testosterone Dodecanoate | 250-300 | 250-300 | 250-300 | 250-300 |
| Maize oil glycerides | 400-500 | 800-1100 | 350-500 | 800-1100 |
| (Maisine 35-1) | | | | |
| Polyoxyl 40 Hydrogenated | 20-25 | 150-213 | 60-69 | 120-144 |
| Castor Oil | | | | |
| (Cremophor RH40) | | | | |
| Glyceryl distearate | — | — | 25-32 | 60-67 |
| (Percirol ATO 5) | | | | |
| Polyethylene Glycol 8000, | 30-39 | — | 39 | — |
| Total mg per capsule | 750-850 | 1200-1500 | 750-850 | 1200-1500 |
| TD-loading (wt %) | 30-35 | 15-20 | 30-35 | 15-20 |
| of capsule fill | | | | |
| TU released in 30 minutes | <75% | <75% | <75% | <75% |
| Time for 75% TU release | <120 | <120 | <120 | <120 |
| (minutes) | | | | |

Note:
TD Examples 27 and 29 can optionally be disposed in delayed release capsule

### TD Examples 30-35

Testosterone dodecanoate formulations TD Examples 30 through 35 can be prepared by using the components set forth in Table XV-TD and by a method similar to that described in TD Example 1. Additionally, indicated amounts of the respective formulations can be encapsulated in gelatin capsules and the testosterone dodecanoate release from the capsules tested in about 1000 mL of 8% w/w solution of Triton X100 in water, maintained at about 37° C., using a USP Type-II apparatus at about 100 rpm. The results of the release testing are also shown in Table XV-TD.

TABLE XV-TD

| | Composition mg/capsule | | | | | |
|---|---|---|---|---|---|---|
| Capsule Components/ | TD Example | TD Example | TD Example | TD Example | TD Example | TD Example |
| Attributes | 30 | 31 | 32 | 33 | 34 | 35 |
| Testosterone | 350-450 | 300-380 | 450-550 | 240-300 | 40-50 | 300-360 |
| Dodecanoate | | | | | | |
| Maize oil glycerides | 800-1000 | 300-400 | 600-650 | 350-450 | — | — |
| (Maisine 35-1) | | | | | | |
| Castor Oil | — | — | — | — | 150-175 | 250-300 |
| Lauroglycol | — | — | | — | 100-115 | 150-200 |
| Tocopherol | | | 80-120 | — | — | |
| Benzyl alcohol | — | — | 80-120 | — | — | |
| Polyoxyl 40 | 40-46 | 20-25 | — | — | — | — |
| Hydrogenated Castor Oil, | | | | | | |
| Polyethylene Glycol | 80-86 | 40-45 | 80-86 | 41-45 | — | — |
| 8000, USP | | | | | | |
| TU-loading (wt %) of | 25-30 | 35-40 | 35-40 | 35-40 | 12-15 | 38-45 |
| capsule fill | | | | | | |
| Total Fill wt. per capsule | 1200-1500 | 700-850 | 1200-1500 | 600-750 | 300-380 | 700-850 |
| TU released in 30 | 62% | <62% | <75% | <75% | ~100% | <75% |
| minutes | | | | | | |
| Time for 75% TU release | <120 | >120 | <120 | <120 | <120 | >120 |
| (minutes) | | | | | | |

TD Examples 30 through 35 demonstrate the importance of the choice of the solubilizers of the current invention and their levels to achieve greater testosterone dodecanoate loading and yet maintain the solubilization of the testosterone dodecanoate in the composition and/or the dosage form.

### TD Examples 36 and 37

The compositions of the current invention can be further adsorbed onto one or more substrate materials such as, for example, lactose, magnesium aluminosilicate, colloidal silicon dioxide, starch, microcrystalline cellulose, alkyl celluloses etc., whereby a free flowing powder/granule form is obtained which can be used as a granules, or disposed into capsule, or pressed into tablet. The amount of the substrate material can be from about 15% to about 40% of the weight of the composition. In one embodiment, the amount of the substrate material can be from about 20% to about 35% of the weight of the formed granule or powder. The method of making such adsorbed testosterone dodecanoate compositions can include pouring the liquid compositions on the substrate material under and continuous mixing at room temperature or at about 50° C.-70° C., depending on the composition. After cooling, the adsorbed composition can be disposed into capsule or pressed into tablet. Table XVI-TD illustrates examples of the freely flowable adsorbed solubilized testosterone dodecanoate compositions.

TABLE XVI-TD

| | Composition | |
|---|---|---|
| | (% w/w) | |
| | TD | TD |
| | Example | Example |
| Components/Attributes | 36 | 37 |
| Testosterone Dodecanoate | 15-20 | 15-20 |
| Maize oil glycerides (Maisine 35-1) | 40-50 | — |
| Castor Oil | — | 40-45 |
| Sorbitan monolaurate (Span ® 20) | — | 3-5 |
| Tocopherol | 0.5-1 | — |
| Glycerylpalmito stearate | 3-5 | — |
| Polyoxyl 40 Hydrogenated Castor Oil, | 1-3 | — |
| Polyethylene Glycol 8000, USP | — | 2-4 |
| Magnesium aluminosilicate | 20-25 | 25-30 |
| (Neusilin ® US2) | | |

### TD Examples 39, 39A, and 40 to 47

Testosterone dodecanoate-containing compositions (capsule fill material) were prepared by using the components set forth in Tables XVIII and XIX. It should be noted that TD Example 40 and 47 are expressed as wt % of TD Examples 17 and 12 respectively. The compositions are prepared by weighing all of the components, except the testosterone dodecanoate, into a clean stainless steel container and mixed together at about 50° C. to about 70° C., using a stirrer. The testosterone dodecanoate (TD) is added and stirred into the mixture of other components until the testosterone dodecanoate dissolves. A predetermined quantity of the “capsule fill” was disposed into a capsule (for example, hard gelatin capsule) to get the required testosterone dodecanoate dose per dosage unit. The capsules are allowed to cool at room temperature, banded (if required) and packaged in a HDPE bottle and tightly closed with an appropriate lid.

TABLE-XVIII-TD

| | TD | TD | TD | TD | TD |
|---|---|---|---|---|---|
| | Example | Example | Example | Example | Example |
| Capsule Components/Attributes | 39 | 39A | 40 | 41 | 42 |
| Testosterone Dodecanoate, wt % | 14-20 | 14-20 | 15-20 | 18-25 | 18-25 |
| Monoglycerides (e.g. maize oil | 65-70 | 70-75 | 60-65 | 65-70 | 65-75 |
| monoglycerides, Maisine 35-1), wt % | | | | | |
| Hydrophilic surfactants (e.g. Cremophor | 10-15 | 5-8 | 15-20 | 5-10 | 10-15 |
| RH40, Cremophor EL), wt % | | | | | |
| Fatty acids (e.g. linoleic acid, | — | — | — | — | — |
| Linolenic acid, Oleic Acid), wt % | | | | | |
| Triglyceride (e.g. castor oil, maize oil, borage | — | — | — | — | — |
| seed oil, lauroglycol, corn oil etc.), wt % | | | | | |
| Lipophilic surfactant (e.g. propylene | — | — | — | — | — |
| glycol monolaurate), wt % | | | | | |
| Solidifying agent (e.g. polyethylene | 4-6 | 4-6 | 4-6 | 4-6 | — |
| glycol 8000) , wt % | | | | | |
| % TU dissolved at RT | 70-75 | 85-92 | 65-70 | 50-55 | 55-60 |
| % TU undissolved fraction at RT | 25-30 | 8-15 | 30-35 | 45-50 | 40-45 |

*It is noted that the compositions can also include one or more components such as anti-oxidants (e.g. BHA, BHT, Ascorbyl palmitate, vitamin E or combinations), flavorings etc.

TABLE-XIX-TD

| | TD | TD | TD | TD | TD |
|---|---|---|---|---|---|
| | Example | Example | Example | Example | Example |
| Capsule Components/Attributes | 43 | 44 | 45 | 46 | 47 |
| Testosterone Dodecanoate, wt % | 25-30 | 18-22 | 22-25 | 12-15 | 12-15 |
| Monoglycerides (e.g. maize oil | 65-70 | — | — | — | — |
| monoglycerides, Maisine 35-1), wt % | | | | | |
| Hydrophilic surfactants (e.g. Cremophor | 3-5 | 15-20 | 15-20 | — | — |
| RH40, Cremophor EL), wt % | | | | | |
| Fatty acids (e.g. linoleic acid, | — | 50-55 | 50-55 | 80-95 | — |
| Linolenic acid, Oleic Acid), wt % | | | | | |
| Triglyceride (e.g. castor oil, maize oil, borage | — | 10-15 | 10-15 | — | 50-60 |
| seed oil, lauroglycol, corn oil etc.), wt % | | | | | |
| Lipophilic surfactant (e.g. propylene | — | — | — | — | 30-35 |
| glycol monolaurate), wt % | | | | | |
| Solidifying agent (e.g. polyethylene | — | — | — | — | — |
| glycol 8000), wt % | | | | | |
| % TU dissolved at RT | 40-45 | 100 | 100 | 100 | 100 |
| % TU undissolved fraction at RT | 55-60 | 0 | 0 | 0 | 0 |

*It is noted that the compositions can also include one or more components such as anti-oxidants (e.g. BHA, BHT, Ascorbyl palmitate, vitamin E or combinations), flavorings etc.

The compositions of TABLES XVIII-TD and XIX-TD include descriptions of the amounts of testosterone dodecanoate that is dissolved at room temperature (RT). The percent (%) of the undissolved testosterone dodecanoate in the composition at RT can range from about 5 wt % to about 60 wt %. It is noteworthy those TD Examples 44 to 47 do not include a solidifying agent and that about 100 wt % of the testosterone dodecanoate is dissolved in these composition. All Examples, except TD Examples 44 to 47 are free of ionizable fatty acids such as oleic acid.

### Testosterone Tridecanoate (TT) Example Compositions

### TT Example 1

A testosterone tridecanoate containing composition was prepared by using the components set forth in Table I-TT. The composition is prepared by weighing all of the components, except the testosterone undecanoate, into a clean stainless steel container and mixed together at about 50° C. to about 70° C., using a stirrer. The testosterone tridecanoate (TT) is added and stirred into the mixture of other components until the testosterone tridecanoate dissolves. A predetermined quantity of this fill material is disposed into a capsule (for example, hard gelatin capsule) to get the required testosterone undecanoate dose per dosage unit. The capsules are allowed to cool at room temperature, banded (if required) and packaged in a HDPE bottle and tightly closed with an appropriate lid.

TABLE I-TT

| | Composition |
|---|---|
| TT Example 1 | mg/capsule |
| Testosterone Tridecanoate | 150-400 |
| Solubilizer (e.g. Glycerides of | 600-800 |
| coconut oil; Capmul ® MCM) | |
| Dispersant (e.g. lauroglycol) | 200-300 |
| Dispersant (polyoxyl 40 | 40-50 |
| hydrogenated castor oil or | |
| Cremophor ® RH40) | |

TT-loading (wt %) of capsule fill = 20-35%

### TT Example 2

A testosterone tridecanoate containing composition was prepared similarly to the method described in Example I-TT using the components set forth in Table II-TT.

TABLE II-TT

| | Composition |
|---|---|
| TT Example 2 | mg/capsule |
| Testosterone Tridecanoate | 150-400 |
| Solubilizer (e.g. Maize | 200-300 |
| oil glyceride) | |
| Dispersant ( e.g. lauroglycol) | 500-700 |

TT-loading (wt %) of capsule fill~15-30%

### TT Examples 3 & 4

Testosterone tridecanoate containing composition were prepared similarly to the method described in Example I-TT using the components set forth in Tables III-TT and

IV-TT.

TABLE III-TT

| | Composition |
|---|---|
| TT Example 3 | mg/capsule |
| Testosterone Tridecanoate | 200-400 |
| Solubilizer (e.g. Glycerides of | 400-700 |
| coconut oil; Capmul ® MCM) | |

TT-loading (wt %) of capsule fill = 25-30%

TABLE IV

| | Composition |
|---|---|
| TT Example 4 | mg/capsule |
| Testosterone Tridecanoate | 180-350 |
| Solubilizer (Maize oil glyceride, | 400-700 |
| Maisine 35-1) | |
| TT-loading (wt %) of capsule fill = 23-25% | |

### TT Example 5

A testosterone tridecanoate containing composition was prepared similarly to the method described in Example I-TT using the components set forth in Table V-TT.

TABLE V-TT

| | Composition |
|---|---|
| TT Composition 5 | mg/capsule |
| Testosterone Tridecanoate | 240-400 |
| Solubilizer (e.g. Glycerides of | 100-250 |
| coconut oil; Capmul ® MCM) | |
| Solubilizer (e.g. α-tocopherol) | 400-550 |
| Dispersant (for e.g. polyoxyl castor | 80-100 |
| oil or Cremophor ® EL) | |
| TT-Loading per caps TU-loading (wt %) of | |
| capsule fill = 23-28% | |

### TT Example 6

A testosterone tridecanoate containing composition was prepared by using the components set forth in Table VI-TT and a method similar to that described in TT Example 1.

TABLE VI-TT

| TT Example 6 | Composition mg/capsule |
|---|---|
| Testosterone Tridecanoate | 200-400 |
| Solubilizer (e.g. Maize oil | 400-550 |
| glycerides) | |
| Dispersant (e.g. polysorbate 80) | 20-30 |
| Solidifying agent (e.g. polyethylene | 40-45 |
| glycol, 8000 or PEG 8000) | |
| TT-Loading per capsule = 25-30% | |

### TT Example 7

A testosterone tridecanoate containing composition was prepared by using the components set forth in Table VII-TT and a method similar to that described in TT Example 1.

TABLE VII-TT

| | Composition |
|---|---|
| TT Example 7 | mg/capsule |
| Testosterone Tridecanoate | 240-400 |
| Solubilizer (e.g.Maize oil glycerides) | 300-350 |
| Solubilizer (e.g. oleic acid) | 100-150 |
| Solubilizer (e.g. Benzyl Alcohol) | 40-50 |
| Solubilizer (e.g. α-tocopherol) | 60-75 |
| Solidifying agent (e.g. PEG 8000) | 40-45 |
| TT-Loading per capsule = 25-35% | |

### TT Example 8

A testosterone tridecanoate containing composition was prepared by using the components set forth in Table VIII-TT and a method similar to that described in TT Example 1.

TABLE VIII-TT

| | Composition |
|---|---|
| TT Example 8 | mg/capsule |
| Testosterone Tridecanoate | 240-400 |
| Solubilizer (e.g. oleic acid) | 300-500 |
| Solidifying agent-(e.g. PEG 8000) | 40-45 |
| TT-loading (wt %) of capsule fill = 30-35% | |

### TT Example 9

A testosterone tridecanoate containing composition was prepared using the components set forth in Table IX-TT and a method similar to that described in TT Example 1.

TABLE IX-TT

| | Composition |
|---|---|
| TT Example 9 | mg/capsule |
| Testosterone Tridecanoate | 240-400 |
| Solubilizer (e.g. Maize oil glycerides) | 300-500 |
| Solubilizer (e.g. α-tocopherol) | 20-24 |
| Solidifying agent-(e.g. Glyceryl | 20-25 |
| distearate ; Percirol ® ATO 5) | |
| TT-loading (wt %) of capsule fill = 33-37% | |

### TT Example 10

A testosterone tridecanoate containing composition can be prepared by using the components set forth in Table X by a method as follows: The required quantity of the glyceryl distearate or glyceryl monostearate and the PEG 8000 are placed in a stainless steel container and heated to about 50 to 70° C. to get a molten mixture. The testosterone tridecanoate is added and stirred till it completely dissolves. A predetermined weight of the molten mixture is disposed into capsules and allowed to congeal at room temperature, banded and packed.

TABLE X-TT

| | Composition |
|---|---|
| TT Example 10 | mg/capsule |
| Testosterone Tridecanoate | 100-200 |
| Glyceryl distearate (Percirol ® ATO 5) or | 150-250 |
| glyceryl monostearate | |
| PEG 8000 | 40-50 |

The oral dosage capsules of TT Example 10, which contains non-dissolved TT, can provide, upon single administration along with food to a human male, a testosterone undecanoate AUC that is about 20% higher as compared to a composition that does not include the glyceryl distearate (Percirol® ATO 5) or glyceryl monostearate. The composition of TT Example 10 can also be optionally modified so that a dispersant such as a disintegrating agent (e.g. Crospovidone at about 150 mg for every 100 mg TT dose) can be uniformly suspended under stirring in the molten testosterone undecanoate solution. This suspension can be further allowed to cooled and passed through ASTM 30 mesh get granulates or particulates which can be either filled in a capsule or compressed to a tablet.

### TT Example 11

A testosterone tridecanoate containing composition wherein at least 50% of the testosterone undecanoate is dissolved is prepared by using the components set forth in Table XI-TT and a method similar to that described under TT Example 1.

TABLE XI-TT

| | Composition |
|---|---|
| TT Example 11 | mg/capsule |
| Testosterone Tridecanoate | 225-350 |
| Solubilizer (e.g. Castor Oil) | 300-400 |
| Dispersant (e.g. lauroglycol); | 150-180 |
| Solidifying agent (e.g. PEG 8000) | 40-45 |
| TT-loading (wt %) of capsule fill = 28-35% | |

### TT Examples 12-19

Testosterone tridecanoate formulations of TT Examples 12 through 19 were prepared by using the components set forth in Table XII-TT and by a method similar to that described under TT Example 1. Additionally, indicated amounts of the respective formulations were filled into hard gelatin capsules and the testosterone tridecanoate release from capsules is measured using a USP Type-II apparatus at about 100 rpm in about 1000 mL of 8% w/w solution of Triton X100 in water, maintained at about 37° C. The results of the release testing are also shown in Table XII-TT.

TABLE XII-TT

| | Composition mg/capsule | | | | | | | | |
|---|---|---|---|---|---|---|---|---|---|
| Capsule | TT | TT | TT | TT | TT | TT | TT | TT | TT |
| Components/ | Example | Example | Example | Example | Example | Example | Example | Example | Example- |
| Attributes | 12 | 13 | 14 | 15 | 15A | 16 | 17 | 18 | 19 |
| Testosterone | 40-80 | 40-80 | 75-150 | 75-150 | 90-180 | 75-150 | 75-150 | 75-150 | 125-250 |
| Tridecanoate | | | | | | | | | |
| Oleic Acid | 200-300 | — | — | — | | — | — | — | |
| Castor oil | | 150-200 | — | — | | 400-500 | — | — | |
| Lauroglycol | | 100-130 | — | — | | — | — | — | |
| Labrafil | | | | | | 60-80 | — | — | |
| M2125CS | | | | | | | | | |
| Maizeoil | | — | 400-500 | 400-500 | 300-350 | — | 300-350 | 300-350 | 350-550 |
| glycerides | | | | | | | | | |
| (Maisine 35-1) | | | | | | | | | |
| Polyoxyl 40 | | — | 120-150 | 60-80 | 30-35 | — | 65-79 | 45-54 | 100-135 |
| Hydrogenated Castor Oil, | | | | | | | | | |
| (Cremophor RH40) | | | | | | | | | |
| Glyceryl distearate | | — | — | 40-50 | 50-60 | — | — | 20-25 | |
| (Percirol ATO 5) | | | | | | | | | |
| Polyethylene | | — | — | — | | 40-50 | 20-30 | 20-30 | 40-50 |
| Glycol 8000, | | | | | | | | | |
| Total mg per | 250-350 | 300-400 | 600-750 | 600-750 | 450-600 | 600-750 | 450-600 | 450-600 | 700-900 |
| capsule (mg) | | | | | | | | | |
| TT-loading (wt %) of | 15-25% | 12-25% | 10-20% | 10-20% | 15-30% | 10-20% | 15-30% | 15-30% | 15-35% |
| capsule fill | | | | | | | | | |
| TT released in | ~100% | ~100% | ~100% | 30% | 30% | ~78% | 85% | 32% | 80% |
| 30 minutes (%) | | | | | | | | | |
| Time for 75% TT release | <120 | <120 | <120 | <120 | <120 | <120 | <120 | <120 | <120 |
| (minutes) | | | | | | | | | |

### TT Examples 20-25

Testosterone tridecanoate formulations of TT Examples 20 through 25 were prepared by using the components set forth in Table XIII and by a method similar to that described under TT Example 1. Additionally, indicated amounts of the respective formulations were filled into hard gelatin capsules and the testosterone tridecanoate release from capsules was tested in about 1000 mL of 8% w/w solution of Triton X100 in water, maintained at about 37° C., using a USP Type-II apparatus at about 100 rpm. The results of the release testing are also shown in Table XIII-TT.

TABLE XIII-TT

| | Composition mg/capsule | | | | | |
|---|---|---|---|---|---|---|
| Capsule Components/ | TT Example | TT Example | TT Example | TT Example | TT Example | TT Example |
| Attributes | 20 | 21 | 22 | 23 | 24 | 25 |
| Testosterone Tridecanoate | 200-400 | 200-400 | 240-400 | 240-400 | 240-400 | 240-400 |
| (TT) | | | | | | |
| Maize oil glycerides | — | 400-550 | 400-550 | 400-550 | | 300-450 |
| (Maisine 35-1) | | | | | | |
| Coconut oil glycerides | — | — | — | — | 300-450 | — |
| (Capmul MCM) | | | | | | |
| Alpha-tocopherol | 400-550 | — | — | 40-50 | — | 40-50 |
| Benzyl alcohol | — | | — | — | 20-25 | 40-50 |
| Polyoxyl 40 Hydrogenated | — | 20-25 | — | — | — | — |
| Castor Oil, | | | | | | |
| Polyoxyl 35 Castor Oil, | 35-45 | — | — | — | — | — |
| Polyethylene Glycol 8000, | 35-45 | 35-45 | — | 35-46 | 55-65 | 30-41 |
| USP | | | | | | |
| Total Fill wt. per capsule | 700-850 | 700-850 | 650-800 | 700-850 | 650-800 | 650-900 |
| (mg) | | | | | | |
| TT-loading (wt %) of | 20-30 | 20-35 | 30-40 | 30-35 | 30-35 | 35-40 |
| capsule fill | | | | | | |
| TT released in 30 minutes | 43% | 62% | 38% | 32% | <75% | <75% |
| Time for 75% TT release | <120 | <120 | <120 | <120 | <120 | <120 |
| (minutes) | | | | | | |

### TT Examples 26-29

Testosterone tridecanoate formulations of TT Examples 26 through 29 were prepared by using the components set forth in Table XIV-TT and a method similar to that described under TT Example I. Additionally, indicated amounts of the respective formulations were filled into hard gelatin capsules and the testosterone tridecanoate released from capsules was tested in about 1000 mL of 8% w/w solution of Triton X100 in water, maintained at about 37° C., using a USP Type-II apparatus at about 100 rpm. The results of the release testing are also shown in Table XIV-TT.

TABLE XIV-TT

| | Composition mg/capsule | | | |
|---|---|---|---|---|
| | TT Example | TT Example | TT Example | TT Example |
| Capsule Components/Attributes | 26 | 27 | 28 | 29 |
| Testosterone Tridecanoate | 250-400 | 250-400 | 250-400 | 250-400 |
| Maize oil glycerides (Maisine 35-1) | 400-550 | 800-1000 | 350-450 | 800-1000 |
| Polyoxyl 40 Hydrogenated Castor | 20-25 | 180-250 | 55-69 | 120-150 |
| Oil (Cremophor RH40) | | | | |
| Glyceryl distearate (Percirol | — | — | 25-32 | 55-67 |
| ATO 5) | | | | |
| Polyethylene Glycol 8000, | 30-39 | — | 30-39 | — |
| Total mg per capsule | 700-850 | 1200-1500 | 700-850 | 1200-1500 |
| TT-loading (wt %) of capsule fill | 30-35 | 15-25 | 30-35 | 15-25 |
| TT released in 30 minutes | <75% | <75% | <75% | <75% |
| Time for 75% TT release (minutes) | <120 | <120 | <120 | <120 |

Note:
TT Examples 27 and 29 can optionally be disposed in a delayed release capsule

### TT Examples 30-35

Testosterone tridecanoate formulations TT Examples 30 through 35 can be prepared by using the components set forth in Table XV-TT and by a method similar to that described in TT Example 1. Additionally, indicated amounts of the respective formulations can be encapsulated in gelatin capsules and the testosterone tridecanoate release from the capsules tested in about 1000 mL of 8% w/w solution of Triton X100 in water, maintained at about 37° C., using a USP Type-II apparatus at about 100 rpm. The results of the release testing are also shown in Table XV-TT.

TABLE XV-TT

| | Composition mg/capsule | | | | | |
|---|---|---|---|---|---|---|
| Capsule Components/ | TT Example | TT Example | TT Example | TT Example | TT Example | TT Example |
| Attributes | 30 | 31 | 32 | 33 | 34 | 35 |
| Testosterone | 350-450 | 320-450 | 450-550 | 240-400 | 40-100 | 300-500 |
| Tridecanoate | | | | | | |
| Maize oil glycerides | 800-1000 | 300-400 | 500-650 | 350-440 | — | — |
| (Maisine 35-1) | | | | | | |
| Castor Oil | — | — | — | — | 150-175 | 250-300 |
| Lauroglycol | — | — | | — | 100-125 | 150-200 |
| Tocopherol | | | 80-102 | — | — | |
| Benzyl alcohol | — | — | 80-102 | — | — | |
| Polyoxyl 40 | 35-46 | 20-25 | — | — | — | — |
| Hydrogenated Castor Oil, | | | | | | |
| Polyethylene Glycol | 70-86 | 40-45 | 70-86 | 35-41 | — | — |
| 8000, USP | | | | | | |
| TT-loading (wt %) of | 25-35 | 35-45 | 35-45 | 35-45 | 10-20 | 38-50 |
| capsule fill | | | | | | |
| Total Fill wt. per capsule | 1200-1500 | 700-850 | 1200-1500 | 600-800 | 250-450 | 700-850 |
| TT released in 30 | 62% | <62% | <75% | <75% | ~100% | <75% |
| minutes | | | | | | |
| Time for 75% TT release | <120 | >120 | <120 | <120 | <120 | >120 |
| (minutes) | | | | | | |

TT Examples 30 through 35 demonstrate the importance of the choice of the solubilizers of the current invention and their levels to achieve greater testosterone tridecanoate loading and yet maintain the solubilization of the testosterone tridecanoate in the composition and/or the dosage form.

### TT Examples 36 and 37

The compositions of the current invention can be further adsorbed onto one or more substrate materials such as, for example, lactose, magnesium aluminosilicate, colloidal silicon dioxide, starch, microcrystalline cellulose, alkyl celluloses etc., whereby a free flowing powder/granule form is obtained which can be used as a granules, or disposed into capsule, or pressed into tablet. The amount of the substrate material can be from about 15% to about 40% of the weight of the composition. In one embodiment, the amount of the substrate material can be from about 20% to about 35% of the weight of the formed granule or powder. The method of making such adsorbed testosterone tridecanoate compositions can include pouring the liquid compositions on the substrate material under and continuous mixing at room temperature or at about 50° C.-70° C., depending on the composition. After cooling, the adsorbed composition can be disposed into capsule or pressed into tablet. Table XVI-TT illustrates examples of the freely flowable adsorbed solubilized testosterone tridecanoate compositions.

TABLE XVI-TT

| | Composition (% w/w) | |
|---|---|---|
| Components/Attributes | TT Example 36 | TT Example 37 |
| Testosterone Tridecanoate | 16-35 | 16-35 |
| Maize oil glycerides (Maisine 35-1) | 40-50 | — |
| Castor Oil | — | 35-45 |
| Sorbitan monolaurate (Sp ® 20) | — | 3-5 |
| Tocopherol | 1-3 | — |
| Glycerylpalmito stearate | 3-5 | — |
| Polyoxyl 40 Hydrogenated Castor | 1-3 | — |
| Oil, | | |
| Polyethylene Glycol 8000, USP | — | 2-4 |
| Magnesium aluminosilicate | 20-25 | 20-30 |
| (Neusilin ® US2) | | |

### TT Examples 39, 39A, and 40 to 47

Testosterone tridecanoate-containing compositions (capsule fill material) were prepared by using the components set forth in Tables XVIII-TT and XIX-TT. It should be noted that TT Example 40 and 47 are expressed as wt % of Examples 17 and 12 respectively. The compositions are prepared by weighing all of the components, except the testosterone tridecanoate, into a clean stainless steel container and mixed together at about 50° C. to about 70° C., using a stirrer. The testosterone tridecanoate (TT) is added and stirred into the mixture of other components until the testosterone tridecanoate dissolves. A predetermined quantity of the “capsule fill” was disposed into a capsule (for example, hard gelatin capsule) to get the required testosterone tridecanoate dose per dosage unit. The capsules are allowed to cool at room temperature, banded (if required) and packaged in a HDPE bottle and tightly closed with an appropriate lid.

TABLE-XVIII-TT

| | TT Example | TT Example | TT Example | TT Example | TT Example |
|---|---|---|---|---|---|
| Capsule Components/Attributes | 39 | 39A | 40 | 41 | 42 |
| Testosterone Tridecanoate, wt % | 14-30 | 14-30 | 15-30 | 18-35 | 18-40 |
| Monoglycerides (e.g. maize oil monoglycerides, Maisine 35-1), wt % | 55-64 | 65-75 | 55-63 | 55-68 | 55-68 |
| Hydrophilic surfactants (e.g. Cremophor RH40, Cremophor EL), wt % | 10-16 | 1-5 | 10-16 | 5-8 | 9-14 |
| Fatty acids (e.g. linoleic acid, Linolenic acid, Oleic Acid), wt % | — | — | — | — | — |
| Triglyceride (e.g. castor oil, maize oil, borage seed oil, lauroglycol, | — | — | — | — | — |
| corn oil etc.), wt % | | | | | |
| Lipophilic surfactant (e.g. propylene glycol monolaurate), wt % | — | — | — | — | — |
| Solidifying agent (e.g. polyethylene glycol 8000), wt % | 6 | 6 | 6 | 6 | — |
| % TT dissolved at RT | 70-75 | 85-92 | 65-70 | 50-55 | 55-60 |
| % TT undissolved fraction at RT | 25-30 | 8-15 | 30-35 | 45-50 | 40-45 |

*It is noted that the compositions can also include one or more components such as anti-oxidants (e.g. BHA, BHT, Ascorbyl palmitate, vitamin E or combinations), flavorings etc.

TABLE-XIX-TT

| | TT | TT | TT | TT | TT |
|---|---|---|---|---|---|
| | Example | Example | Example | Example | Example |
| Capsule Components/Attributes | 43 | 44 | 45 | 46 | 47 |
| Testosterone Tridecanoate, wt % | 25-45 | 18-25 | 25-40 | 12-25 | 12-25 |
| Monoglycerides (e.g. maize oil monoglycerides, Maisine 35-1), wt % | 60-70 | — | — | — | — |
| Hydrophilic surfactants (e.g. Cremophor RH40, Cremophor EL), wt % | 3-5 | 10-17 | 10-16 | | — |
| Fatty acids (e.g. linoleic acid, Linolenic acid, Oleic Acid), wt % | — | 45-53 | 45-52 | 75-88 | — |
| Triglyceride (e.g. castor oil, maize oil, borage seed oil, lauroglycol, | — | 5-12 | 5-12 | — | 40-53 |
| corn oil etc.), wt % | | | | | |
| Lipophilic surfactant (e.g. propylene glycol monolaurate), wt % | — | — | — | — | 30-35 |
| Solidifying agent (e.g. polyethylene glycol 8000) , wt % | — | — | — | — | — |
| % TT dissolved at RT | 40-45 | 100 | 100 | 100 | 100 |
| % TT undissolved fraction at RT | 55-60 | 0 | 0 | 0 | 0 |

*It is noted that the compositions can also include one or more components such as anti-oxidants (e.g. BHA, BHT, Ascorbyl palmitate, vitamin E or combinations), flavorings etc.

The compositions of Tables XVIII-TT and XIX-TT include descriptions of the amounts of testosterone tridecanoate that is dissolved at room temperature (RT). The percent (%) of the undissolved testosterone tridecanoate in the composition at RT can range from about 5 wt % to about 60 wt %. It is noteworthy those TT Examples 44 to 47 do not include a solidifying agent and that about 100 wt % of the testosterone tridecanoate is dissolved in these composition. All Examples, except TT Examples 44 to 47 are free of ionizable fatty acids such as oleic acid.

### TU

| Composition | 15a | 15b | 15c | 40 | 41 | 45 | 47 |
|---|---|---|---|---|---|---|---|
| Testosterone | 150 | 360 | 1500 | | | | |
| | mg | mg | mg | | | | |
| Maize oil glycerides | 910 | 1260 | 9100 | 63% | | | |
| (Maisine 35-1) | mg | mg | mg | | | | |
| Polyoxyl 40 | 260 | 140 | 2600 | | | | |
| | mg | mg | mg | | | | |
| Glyceryl distearate | 100 | 240 | 1000 | | | | |
| (Percirol ATO 5) | mg | mg | mg | | | | |
| Polyethylene | | | | | | | |
| Glycol 8000 | | | | | | | |
| TU, wt % | | | | 15% | 18% | 20% | 12% |
| Hydrophilic surfactants | | | | 16% | 8% | 16% | |
| (e.g. Cremophor RH40, | | | | | | | |
| Cremophor EL), wt % | | | | | | | |
| Maize oil glycerides | | | | 63% | 68% | | |
| (Maisine 35-1) | | | | | | | |
| Solidifying agent (e.g. | | | | 6% | 6% | | |
| polyethylene glycol 8000), | | | | | | | |
| wt % | | | | | | | |
| Fatty acids (e.g. linoleic | | | | | | 52% | |
| acid, Linolenic acid, | | | | | | | |
| Oleic Acid), wt % | | | | | | | |
| Triglyceride (e.g. castor oil, | | | | | | 12% | 52% |
| maize oil, borage seed oil, | | | | | | | |
| lauroglycol, corn oil etc.), | | | | | | | |
| wt % | | | | | | | |
| Lipophilic Surfactant (e.g. | | | | | | | 35% |
| propylene glycol | | | | | | | |
| monolaurate), wt % | | | | | | | |

The mean marker change from baseline in the foregoing tables can be measured at various times including: hours after treatment commences, 7 to 14 days after treatment commences, and during treatment.

| Composition | 15a | 15b | 15c | 40 | 41 | 45 | 47 |
|---|---|---|---|---|---|---|---|
| Total daily | 150 | 360-460 | 1500 | 430-460 | 430-460 | 600-650 | 240 |
| TU dose | | | | | | | |
| administered, | | | | | | | |
| mg | | | | | | | |
| Cmax to | 1.54-2.69 | 1.54-2.69 | 1.54-2.69 | 1.54-2.69 | 1.54-2.69 | 1.93 | >2.84 |
| mean Cave | | | | | | | |
| ratio | | | | | | | |
| Mean marker | −5% to −35% | −5% to −35% | −5% to −35% | −5% to −35% | −5% to −35% | −5% to −35% | −5% to −35% |
| change from | | | | | | | |
| baseline in | | | | | | | |
| serum (e.g. | | | | | | | |
| IL-2, IL-6, | | | | | | | |
| TNF-Alpha, | | | | | | | |
| CRP, SHBG, | | | | | | | |
| MCP-1, AST, | | | | | | | |
| ALT, ALP) | | | | | | | |

### TT

A-E are all the 14-day timepoint

| Attribute | TT A | TT B | TT C | TT D | TT E | TT | TT |
|---|---|---|---|---|---|---|---|
| Total daily | 500 | 750 | 1000 | 1250 | 500 | 300 | 2700 |
| TT dose | | | | | | | |
| administered, | | | | | | | |
| mg | | | | | | | |
| Cmax to | 1.54-2.69 | 1.69-4.47 | 1.97-3.21 | 2.15-2.91 | 1.23-2.10 | 1.72-3.08* | 1.72-3.09* |
| mean Cave | | | | | | | |
| ratio | | | | | | | |
| Mean marker | −5% to −35% | −5% to −35% | −5% to −35% | −5% to −35% | −5% to −35% | −5% to −35% | −5% to −35% |
| change from | | | | | | | |
| baseline in | | | | | | | |
| serum (e.g. | | | | | | | |
| IL-2, IL-6, | | | | | | | |
| TNF-Alpha, | | | | | | | |
| CRP, SHBG, | | | | | | | |
| MCP-1, AST, | | | | | | | |
| ALT, ALP) | | | | | | | |

*range calculated from the average low and average high for the 5 arms tested.

### TD—Single Dose

| Attribute | TD | TD | TD | TD |
|---|---|---|---|---|
| Total daily TD dose | 155 | 200 | 900 | 1800 |
| administered, mg | | | | |
| Cmax to mean Cave | 2.03- | 2.03- | 2.03- | 2.03- |
| ratio | 2.81 | 2.82** | 2.83** | 2.84** |
| Mean marker change | −5% to | −5% to | −5% to | −5% to |
| from baseline in | −35% | −35% | −35% | −35% |
| serum (e.g. IL-2, IL-6, | | | | |
| TNF-Alpha, CRP, | | | | |
| SHBG, MCP-1, AST, | | | | |
| ALT, ALP) | | | | |

**Linearity was assumed, so range is constant

TU Depot Injection—after Single Dose

| Attribute | TD | TD |
|---|---|---|
| Total TU dose | 500 | 1000 |
| administered, mg | | |
| Cmax to mean Cave ratio | 0.94 (0.78-1.06) | 0.70 (0.65-0.73) |

Topical T—after 7 Days

| Topical | Formulation A | Formulation B |
|---|---|---|
| Total T dose | 50 | 50 |
| administered, mg | | |
| Cmax to mean Cave ratio | 1.96 (1.60-2.22) | 1.96 (1.67-2.25) |

It is understood that the above-described various types of compositions, dosage forms and/or modes of applications are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, but not limited to, variations in size, materials, shape, form, function and manner of operation, assembly and use may be made without departing from the principles and concepts set forth herein.

1. A method of treating COVID-19 in a subject comprising:
administering a therapeutically effective amount of a testosterone agent to the subject.

2. The method of claim 1, wherein the subject has an acute state of COVID-19, or a chronic state of COVID-19.

3. The method of claim 1, wherein the subject has a comorbid condition selected from the group consisting of: cardiovascular disease, obesity, diabetes, chronic respiratory disease, hypertension, thyroid dysfunction, chronic kidney disease, liver disease, osteoporosis, hypogonadism, cancer, sleep apnea, frailty, blood coagulation disorder, sarcopenia, cachexia, and combinations thereof.

4. The method of claim 1, wherein the subject displays either substantially no COVID-19 symptoms, mild COVID-19 symptoms, moderate COVID symptoms, severe COVID-19 symptoms, or critical COVID-19 symptoms.

5. The method of claim 1, wherein the subject displays acute respiratory distress syndrome (ARDS), pneumonia, or a combination thereof.

6. The method of claim 1, further comprising administering the therapeutically effective amount of the testosterone agent to the subject according to a dosage regimen of at least once per day for a specified duration of from about a single day to about 3 months.

7. The method of claim 1, wherein the testosterone agent is administered to the subject via an administration route that increases free testosterone serum levels compared to a free testosterone baseline level, or decreases sex hormone binding globulin (SHBG) serum levels compared to an SHBG baseline level, or a combination thereof.

8. The method of claim 7, wherein the decrease in SHBG is a decrease of from about 5% to about 60%.

9. The method of claim 1 wherein the testosterone agent is orally administered to the subject.

10. The method of claim 9, wherein the therapeutically effective amount of the testosterone agent is orally administered once per day or twice per day.

11. The method of claim 9, wherein the testosterone agent is testosterone, a testosterone derivative, a testosterone ester, or a combination of testosterone esters.

12. The method of claim 11, wherein the testosterone ester is a member of a group consisting of: testosterone undecanoate, testosterone dodecanoate, testosterone tridecanoate, and combinations thereof.

13. The method of claim 11, wherein a daily dose of the therapeutically effective amount of the testosterone ester comprises:
from about 150 mg to about 1500 mg when the testosterone ester is testosterone undecanoate and the subject is male;
from about 10 mg to about 150 mg when the testosterone ester is testosterone undecanoate and the subject is female;
from about 150 mg to about 1800 mg when the testosterone ester is testosterone dodecanoate and the subject is male;
from about 15 mg to about 180 mg when the testosterone ester is testosterone dodecanoate and the subject is female;
from about 300 mg to about 2700 mg when the testosterone ester is testosterone tridecanoate and the subject is male; and
from about 20 mg to about 270 mg when the testosterone ester is testosterone tridecanoate and the subject is female.

14. The method of claim 1, wherein administration of the therapeutically effective amount of the testosterone agent to the subject:
results in a maintenance or an increase in a level of a T/E2 ratio.

15. The method of claim 1, wherein administration of the therapeutically effective amount of the testosterone agent to the subject results in a decrease of a pro-inflammatory cytokine and/or a pro-inflammatory chemokine, wherein:
the pro-inflammatory cytokine is a member of the group consisting of: interleukin (IL-1), IL-6, IL-8, IL-12, gamma interferon (IFN-γ), IL-18, tumor necrosis factor (TNF) or a combination thereof; and
the pro-inflammatory chemokine comprises a monocyte chemoattractant protein-1 (MCP-1).

16. A method of treating a virally-induced respiratory condition in a subject comprising:
administering a therapeutically effective amount of an androgen receptor agonist to the subject.

17. The method of claim 16, wherein the subject has an acute state of the virally-induced respiratory condition, or has a chronic state of the virally-induced respiratory condition.

18. The method of claim 16, wherein the subject has a comorbid condition selected from the group consisting of: cardiovascular disease, obesity, diabetes, chronic respiratory disease, hypertension, thyroid dysfunction, chronic kidney disease, liver disease, osteoporosis, hypogonadism, cancer, sleep apnea, frailty, blood coagulation disorder, sarcopenia, cachexia, and combinations thereof.

19. The method of claim 16, wherein the virally-induced respiratory condition is a member selected from the group consisting of: severe acute respiratory syndrome coronavirus (SARS-CoV-1), severe acute respiratory syndrome coronavirus 2 (SARS-CoV-2), Middle East respiratory syndrome (MERS), influenza, and H1N1.

20. The method of claim 16, wherein the viral infection is a member selected from the group of families consisting of: Coronoviridae, Orthomyxoviridae, Paramyxoviridae, Picornaviridae, Adenoviridae, and Parvoviridae.

21. The method of claim 16, wherein the virus is a member of the group consisting of: SARS-COV1, SARS-COV2, MERS, H1N1, and influenza.

22. The method of claim 16, wherein the subject displays mild respiratory symptoms, moderate respiratory symptoms, severe respiratory symptoms, or critical respiratory symptoms.

23. The method of claim 16, wherein the virally-induced respiratory condition results in acute respiratory distress syndrome (ARDS) or pneumonia.

24. The method of claim 16, wherein the androgen receptor agonist is orally administered to the subject.

25. The method of claim 16, wherein the therapeutically effective amount of the androgen receptor agonist is orally administered once per day or twice per day.

26. The method of claim 16, wherein the androgen receptor agonist is a member selected from the group consisting of: testosterone, a testosterone ester, dehydroepiandrosterone (DHEA), dehydroepiandrosterone sulfate (DHEA-S), androstenedione (A4), Androsterone, androstenediol (A5), nandrolone, dimethandrolone, dihydrotestosterone, and dihydrotestosterone esters.

27. The method of claim 16, further comprising:
identifying a subject in need of treatment.

28. The method of claim 16, wherein administration of the therapeutically effective amount of the androgen receptor agonist to the subject:
improves clinical symptoms associated with lung function; or
improves clinical outcomes associated with morbidity; or
improves clinical outcomes associated with survival.

29. The method of claim 16, wherein administration of the therapeutically effective amount of the androgen receptor agonist to the subject results in a decrease of a pro-inflammatory cytokine and/or a pro-inflammatory chemokine, wherein:
the pro-inflammatory cytokine is a member selected from the group consisting of: interleukin (IL-1), IL-6, IL-8, IL-12, gamma interferon (IFN-γ), IL-18, tumor necrosis factor (TNF); and
the proinflammatory chemokine is monocyte chemoattractant protein-1 (MCP-1).

30. A method of treating an acute state of COVID-19 in a subject, comprising:
administering a therapeutically effective amount of an androgen receptor agonist to the subject.
